(19) United States
(12) Patent Application Publication
(10) Pub. No.: US 2022/0144738 A1
(43) Pub. Date: May 12, 2022

(54) COMPOUND, RESIN, COMPOSITION, RESIST PATTERN FORMATION METHOD, CIRCUIT PATTERN FORMATION METHOD, AND METHOD FOR PURIFYING RESIN

(71) Applicant: Mitsubishi Gas Chemical Company, Inc., Tokyo (JP)

(72) Inventors: Takashi MAKINOSHIMA, Hiratsuka-shi, Kanagawa (JP); Yu OKADA, Hiratsuka-shi, Kanagawa (JP); Hiroaki YAMAMOTO, Hiratsuka-shi, Kanagawa (JP); Masatoshi ECHIGO, Chiyoda-ku, Tokyo (JP)

(21) Appl. No.: 17/426,978

(22) PCT Filed: Jan. 31, 2020
(86) PCT No.: PCT/JP2020/003740

§ 371 Date: Jul. 29, 2021

(30) Foreign Application Priority Data
Jan. 31, 2019 (JP) 2019-015639

(51) Int. Cl. C07C 39/16; G03F 7/038; G03F 7/38; G03F 7/32; G03F 7/20; G03F 7/11; G03F 7/004; G03F 7/40; G03F 7/16; C08G 8/10

(52) U.S. Cl. CPC C07C 39/16; G03F 7/0382; G03F 7/38; G03F 7/322; C08G 8/10; G03F 7/11; G03F 7/0045; G03F 7/40; G03F 7/162; G03F 7/2004

(57) ABSTRACT

The present invention has an object to provide a new compound that is useful as a film forming material for lithography or an optical component forming material, a resin containing a constituent unit derived from said compound, a composition, a resist pattern formation method, a circuit pattern formation method, and a purification method.

A compound represented by formula (1), a resin containing a constituent unit derived from said compound, a composition containing one or more selected from the group consisting of said compound and said resin, a resist pattern formation method using said composition, a circuit pattern formation method, and a purification method thereof.

### TECHNICAL FIELD

The present invention relates to a compound, a resin, a composition, a resist pattern formation method, a circuit pattern formation method, and a method for purifying the resin.

### BACKGROUND ART

In the production of semiconductor devices, fine processing is practiced by lithography using photoresist materials. In recent years, further miniaturization based on pattern rules has been demanded along with increase in the integration and speed of LSIs (large scale integrated circuits). The light source for lithography used upon forming resist patterns has been shifted to ArF excimer laser (193 nm) having a shorter wavelength from KrF excimer laser (248 nm). The introduction of extreme ultraviolet (EUV, 13.5 nm) is also expected.

However, because conventional polymer-based resist materials have a molecular weight as large as about 10,000 to 100,000 and also wide molecular weight distribution, in lithography using such a polymer-based resist material, roughness occurs on a pattern surface; the pattern dimension becomes difficult to be controlled; and there is a limitation in miniaturization. Accordingly, various low molecular weight resist materials have been proposed so far in order to provide resist patterns having higher resolution. The low molecular weight resist materials are expected to provide resist patterns having high resolution and small roughness, because of their small molecular sizes.

Various materials are currently known as such low molecular weight resist materials. For example, an alkaline development type negative type radiation-sensitive composition (see, for example, Patent Literature 1 and Patent Literature 2) using a low molecular weight polynuclear polyphenolic compound as a main component has been suggested; and as a candidate of a low molecular weight resist material having high heat resistance, an alkaline development type negative type radiation-sensitive composition (see, for example, Patent Literature 3 and Non Patent Literature 1) using a low molecular weight cyclic polyphenolic compound as a main component has been suggested as well. Also, as a base compound of a resist material, a polyphenolic compound is known to be capable of imparting high heat resistance despite a low molecular weight and useful for improving the resolution and roughness of a resist pattern (see, for example, Non Patent Literature 2).

In addition, in Patent Literature 4, a resist composition containing a compound having a specific structure and an organic solvent has been proposed as a material that is excellent in etching resistance and is also soluble in a solvent and applicable to a wet process.

Also, as the miniaturization of resist patterns proceeds, the problem of resolution or the problem of collapse of resist patterns after development arises. Therefore, thinner resist films have been desired. However, if resist films are merely made thinner, it is difficult to obtain resist patterns with sufficient film thicknesses for substrate processing. Therefore, there has been a need for a process of preparing an underlayer film between a resist and a semiconductor substrate to be processed, and imparting, also to this underlayer film, functions as a mask for substrate processing in addition to a resist pattern.

Various underlayer films for such lithography are currently known. For example, as a material for realizing resist underlayer films having the selectivity of a dry etching rate close to that of resists, unlike conventional underlayer films having a fast etching rate, an underlayer film forming material for a multilayer resist process containing a solvent and a resin component having at least a substituent that generates a sulfonic acid residue by eliminating a terminal group under application of predetermined energy has been suggested (see Patent Literature 5). Also, in order to realize an underlayer film for lithography having the selectivity of a dry etching rate smaller than that of resists, an underlayer film material comprising a polymer having a specific repeat unit has been suggested (see Patent Literature 6). Furthermore, as a material for realizing underlayer films for lithography having the selectivity of a dry etching rate slower than that of semiconductor substrates, a resist underlayer film material comprising a polymer prepared by copolymerizing a repeat unit of an acenaphthylene and a repeat unit having a substituted or unsubstituted hydroxy group has been suggested (see Patent Literature 7).

Meanwhile, as materials having high etching resistance for this kind of resist underlayer film, amorphous carbon underlayer films formed by chemical vapor deposition (CVD) using methane gas, ethane gas, acetylene gas, or the like as a raw material are well known. However, resist underlayer film materials that can form resist underlayer films by a wet process such as spin coating or screen printing have been demanded from the viewpoint of processability.

In addition, Patent Literature 8 describes an underlayer film forming material for lithography containing a compound having a specific structure as a material that is excellent in etching resistance, has high heat resistance, and is soluble in a solvent and applicable to a wet process.

As for methods for forming an intermediate layer used in the formation of a resist underlayer film in a three-layer process, for example, a method for forming a silicon nitride film (see Patent Literature 9) and a CVD formation method for a silicon nitride film (see Patent Literature 10) are known. Also, as intermediate layer materials for a three-layer process, materials comprising a silsesquioxane-based silicon compound are known (see Patent Literature 11 and Patent Literature 12).

Various compositions have been further proposed as optical component forming compositions. For example, Patent Literature 13 discloses an energy beam curable resin composition for optical lens sheets comprising: an ionic liquid; a compound having a predetermined polyalkylene oxide structure and a (meth)acryloyl group; a predetermined (meth)acrylate monomer; and a photopolymerization initiator. Patent Literature 14 indicates that a resin composition containing: a copolymer having a specific structural unit; a specific curing-accelerating catalyst; and a solvent is suitably used for microlenses or for flattening films.

### CITATION LIST

### Patent Literature

Patent Literature 1: Japanese Patent Laid-Open No. 2005-326838
Patent Literature 2: Japanese Patent Laid-Open No. 2008-145539
Patent Literature 3: Japanese Patent Laid-Open No. 2009-173623
Patent Literature 4: International Publication No. WO 2013/024778
Patent Literature 5: Japanese Patent Laid-Open No. 2004-177668
Patent Literature 6: Japanese Patent Laid-Open No. 2004-271838
Patent Literature 7: Japanese Patent Laid-Open No. 2005-250434
Patent Literature 8: International Publication No. WO 2013/024779
Patent Literature 9: Japanese Patent Laid-Open No. 2002-334869
Patent Literature 10: International Publication No. WO 2004/066377
Patent Literature 11: Japanese Patent Laid-Open No. 2007-226170
Patent Literature 12: Japanese Patent Laid-Open No. 2007-226204
Patent Literature 13: Japanese Patent Laid-Open No. 2010-138393
Patent Literature 14: Japanese Patent Laid-Open No. 2015-174877

### Non Patent Literature

Non Patent Literature 1: T. Nakayama, M. Nomura, K. Haga, M. Ueda: Bull. Chem. Soc. Jpn., 71, 2979 (1998)
Non Patent Literature 2: Shinji Okazaki et al., “New Trends of Photoresists,” CMC Publishing Co., Ltd., September 2009, pp. 211-259

### SUMMARY OF INVENTION

### Technical Problem

However, it has been required for film forming materials for lithography or optical component forming materials to have high levels of solubility in organic solvents, etching resistance, and resist pattern formability at the same time.

Therefore, the present invention has an object to provide a new compound that is useful as a film forming material for lithography or an optical component forming material, a resin containing a constituent unit derived from said compound, a composition, a resist pattern formation method, a circuit pattern formation method, and a purification method.

### Solution to Problem

The present inventors have, as a result of devoted examinations to solve the problems described above, found out that a new compound having a specific structure can be obtained and that said new compound is useful as a film forming material for lithography or an optical component forming material, leading to completion of the present invention.

More specifically, the present invention is as follows.

[1]

A compound represented by the following formula (1):

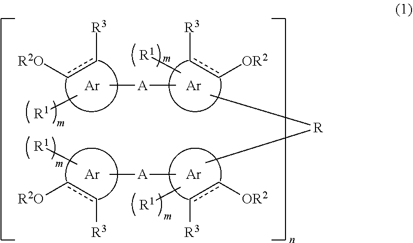

wherein
each A is independently a single bond or a linking group;
Ar is an aromatic ring;
R is a 2n-valent group having 1 to 60 carbon atoms and optionally having a substituent and/or a heteroatom;
each R¹ is independently a linear, branched, or cyclic alkyl group having 1 to 30 carbon atoms, an aryl group having 6 to 40 carbon atoms, an alkenyl group having 2 to 30 carbon atoms, an alkynyl group having 2 to 30 carbon atoms, a halogen atom, a nitro group, an amino group, a carboxy group, a cyano group, a mercapto group, or a hydroxy group;
each R² is independently a hydrogen atom, a crosslinkable group, a dissociable group, a linear, branched, or cyclic alkyl group having 1 to 30 carbon atoms, or an aryl group having 6 to 40 carbon atoms;
provided that at least one R² is any of a hydrogen atom, a crosslinkable group, and a dissociable group;
each R³ is independently a linear, branched, or cyclic alkyl group having 1 to 30 carbon atoms, an aryl group having 6 to 40 carbon atoms, an alkenyl group having 2 to 30 carbon atoms, an alkynyl group having 2 to 30 carbon atoms, a halogen atom, a nitro group, an amino group, a carboxy group, a cyano group, a mercapto group, or a hydroxy group;
each m is independently an integer of 0 to 8;
n is an integer of 1 to 4;
each p is independently an integer of 0 to 3; and
the alkyl group, the aryl group, the alkenyl group, and the alkynyl group optionally have a substituent and/or a heteroatom,
provided that a compound represented by the following formula (A) is excluded:

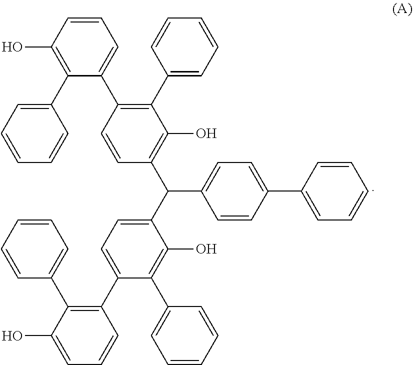 [SMILES: Oc1cccc(-c2ccc(C(c3ccc(-c4ccccc4)cc3)c3ccc(-c4cccc(O)c4-c4ccccc4)c(-c4ccccc4)c3O)c(O)c2-c2ccccc2)c1-c1ccccc1]

[2]

The compound according to the above [1] represented by the following formula (1-1):

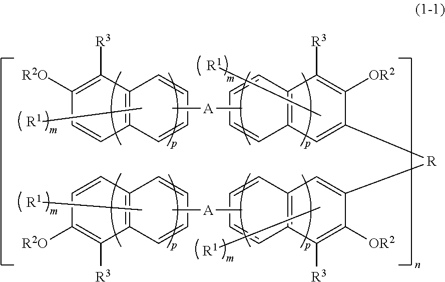

wherein
A, R, R¹ to R³, n, and m are each as defined in the formula (1); and
each p is independently an integer of 0 to 3.
   [3]

The compound according to the above [2], wherein each R² is independently a hydrogen atom, a linear, branched, or cyclic alkyl group having 1 to 30 carbon atoms, or an aryl group having 6 to 40 carbon atoms, and at least one R² is a hydrogen atom.

[4]

The compound according to the above [2] or [3], wherein when p is 0, a substitution position of A is a para position with respect to the R²O— group.

[5]

The compound according to any one of the above [1] to [4], wherein the compound represented by the formula (1) is a compound represented by the following formula (1a):

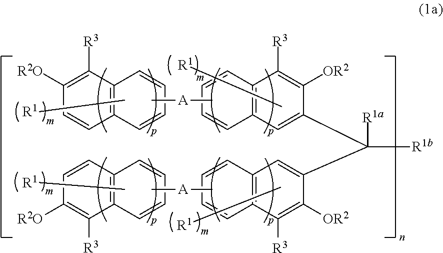

wherein
A, R¹ to R³, n, m, and p are each as defined in the formula (1) or the formula (1-1);
R^{1a} is a hydrogen atom or a monovalent group having 1 to 10 carbon atoms;
R^{1b} is an n-valent group having 1 to 30 carbon atoms;
R^{1a} and R^{1b} may bind to each other to form a cyclic group having 2 to 40 carbon atoms; and
the monovalent group and the n-valent group optionally have a substituent and/or a heteroatom.
   [6]

The compound according to the above [5], wherein the compound represented by the formula (1a) is a compound represented by the following formula (1b):

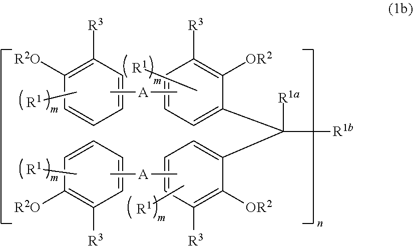

wherein
A, R¹ to R³, R^{1a}, R^{1b}, n, and m are each as defined in the formula (1) or the formula (1a)
   [7]

The compound according to the above [6], wherein the compound represented by the formula (1b) is a compound represented by the following formula (1c):

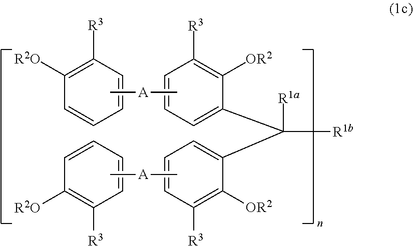

wherein
A, R², R³, R^{1a}, R^{1b}, and n are each as defined in the above formula (1) or the formula (1a).
   [8]

The compound according to any one of the above [5] to [7], wherein all R² is a hydrogen atom in the formulae (1a) to (1c).

[9]

The compound according to any one of the above [5] to [8], wherein all R³ is a methyl group in the above formulae (1a) to (1c).

[10]

The compound according to the above [6], wherein the compound represented by the formula (1b) is a compound represented by the following formula (1d-1):

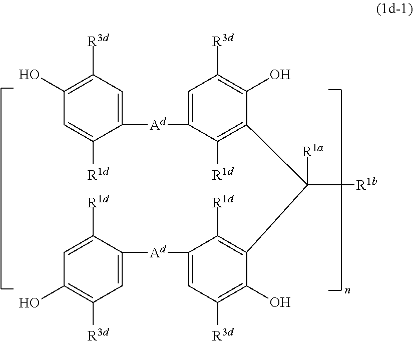

wherein
R^{1a}, R^{1b}, and n are each as defined in the formula (1) or the formula (1a); each R^{3d} is independently a linear or branched alkyl group having 1 to 4 carbon atoms or a phenyl group; each R^{1d} is independently a hydrogen atom or a linear or branched alkyl group having 1 to 4 carbon atoms; and A^{d} is a single bond, a methylene group, or a 2,2-propanediyl group.
   [11]

The compound according to the above [10], wherein the compound represented by the formula (1c) is a compound represented by the following formula (1d-1a):

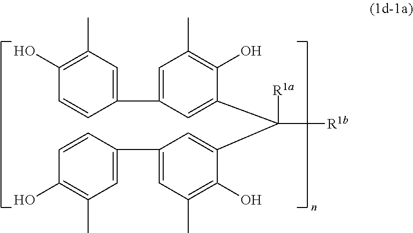

wherein
R^{1a}, R^{1b}, and n are each as defined in the formula (1) or the formula (1a).
   [12]

The compound according to the above [6], wherein the compound represented by the formula (1b) is a compound represented by the following formula (1d-2):

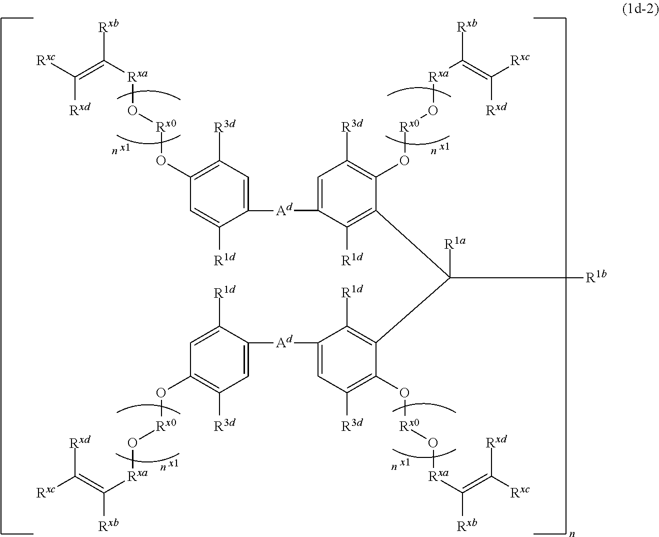

wherein R^{1a}, R^{1b}, and n are each as defined in the formula (1) or the formula (1a); R^{3d}, R^{1d}, and A^{d} are each as defined in the formula (1d-1); R^{x0} is an ethylene group or a propylene group; n^{x1} is 0 to 5; R^{xa} is a single bond or a linking group; and R^{xb}, R^{xc}, and R^{xd} are each independently a hydrogen atom or a methyl group.
[13]

The compound according to the above [6], wherein the compound represented by the formula (1b) is a compound represented by the following formula (1d-3):

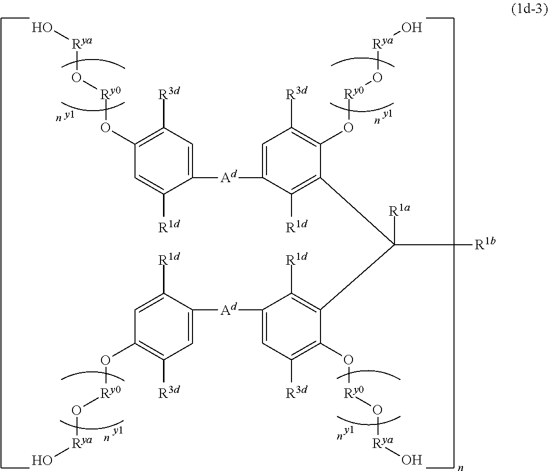

wherein R^{1a}, R^{1b}, and n are each as defined in the formula (1) or the formula (1a); R^{3d}, R^{1d}, and A^{d} are each as defined in the formula (1d-1); R^{y0} is an ethylene group or a propylene group; n^{y1} is 0 to 5; and R^{ya} is a divalent aliphatic hydrocarbon group having 1 to 3 carbon atoms.
[14]

The compound according to the above [6], wherein the compound represented by the formula (1b) is a compound represented by the following formula (1d-4):

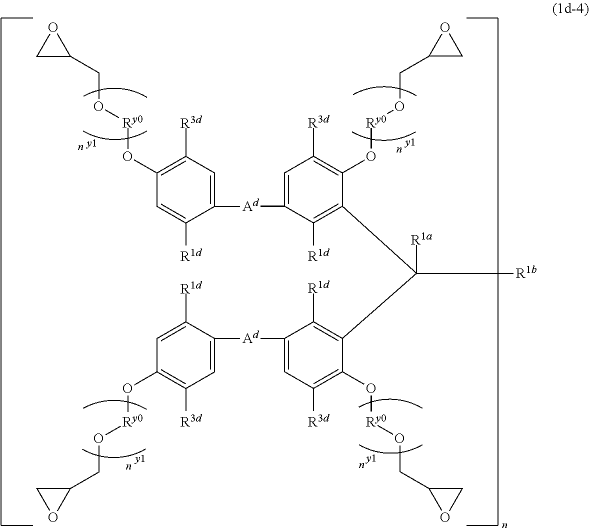

wherein

R^{1a}, R^{1b}, and n are each as defined in the formula (1) or the formula (1a); R^{3d}, R^{1d}, and A^{d} are each as defined in the formula (1d-1); and R^{y0} and n^{y1} are each as defined in the formula (1d-3).

[15]

The compound according to the above [6], wherein the compound represented by the formula (1b) is a compound represented by the following formula (1d-5):

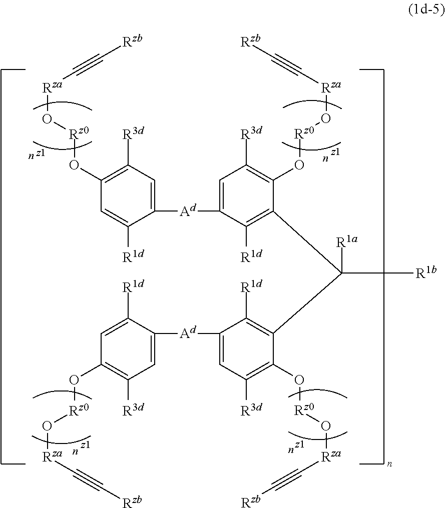

wherein

R^{1a}, R^{1b}, and n are each as defined in the formula (1) or the formula (1a); R^{3d}, R^{1d}, and A^{d} are each as defined in the formula (1d-1); R^{z0} is an ethylene group or a propylene group; n^{z1} is 0 to 5; R^{za} is a single bond or a linking group; and R^{zb} is a hydrogen atom or a monovalent hydrocarbon group having 1 to 20 carbon atoms.

[16]

The compound according to the above [6], wherein the compound represented by the formula (1b) is a compound represented by the following formula (1d-6):

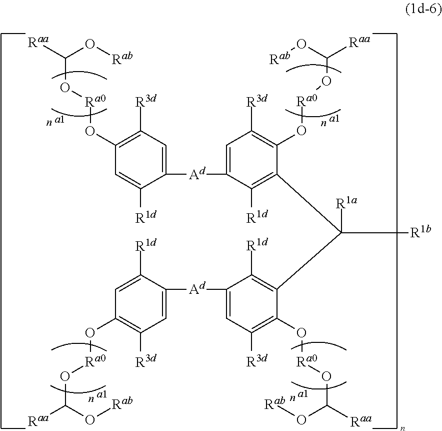

wherein R^{1a}, R^{1b}, and n are each as defined in the formula (1) or the formula (1a); R^{3d}, R^{1d}, and A^{d} are each as defined in the formula (1d-1); R^{a0} is an ethylene group or a propylene group; n^{a1} is 0 to 5; and R^{aa} is a hydrogen atom or a linear, branched, or cyclic alkyl group having 1 to 30 carbon atoms; and R^{ab} is a linear, branched, or cyclic alkyl group having 1 to 30 carbon atoms.
[17]

The compound according to the above [6], wherein the compound represented by the formula (1b) is a compound represented by the following formula (1d-7):

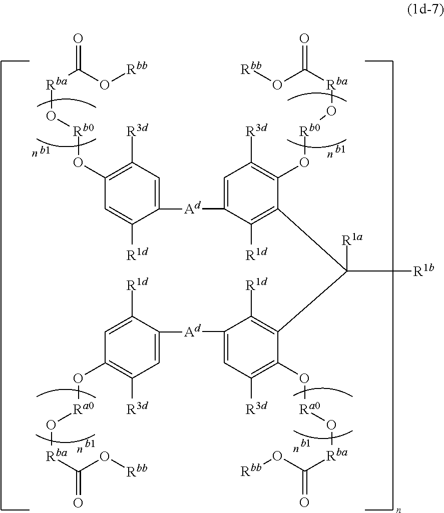

wherein R^{1a}, R^{1b}, and n are each as defined in the formula (1) or the formula (1a); R^{3d}, R^{1d}, and A^{d} are each as defined in the formula (1d-1); R^{b0} is an ethylene group or a propylene group; n^{b1} is 0 to 5; and R^{ba} is a single bond or a linking group; and R^{bb} is a linear, branched, or cyclic alkyl group having 1 to 30 carbon atoms.
[18]

A resin containing a constituent unit derived from the compound according to any one of the above [1] to [17].

[19]

The resin according to the above [18], having a structure represented by the following formula (2):

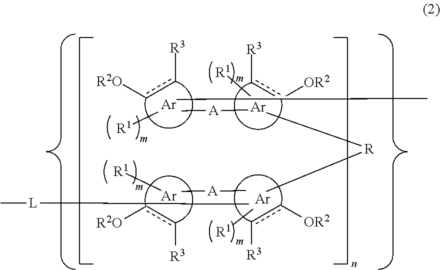

wherein A, R, R¹ to R³, m, n, and p are each as defined in the formula (1); and
L is a single bond or a linking group.
   [20]

The resin according to the above [19], having a structure represented by the following formula (2-1):

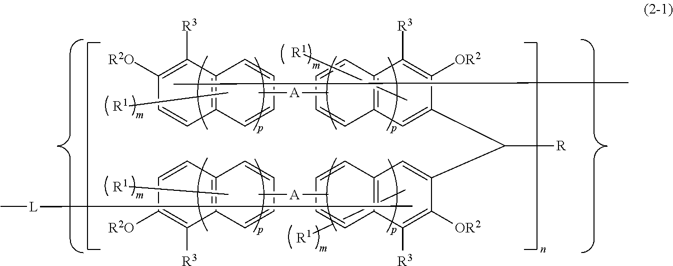

wherein A, R, R¹ to R³, m, n, and p are each as defined in the formula (1) or the formula (1-1); and
L is a single bond or a linking group.
   [21]

A composition comprising one or more selected from the group consisting of the compound according to any one of the above [1] to [17] and the resin according to any one of the above [18] to [20].

[22]

The composition according to the above [21], further comprising a solvent.

[23]

The composition according to the above [21] or [22], further comprising an acid generating agent.

[24]

The composition according to any one of the above [21] to [23], further comprising a crosslinking agent.

[25]

The composition according to any one of the above [21] to [24], further comprising a crosslinking promoting agent.

[26]

The composition according to any one of the above [21] to [25], which is used in film formation for lithography.

[27]

The composition according to the above [26], which is used in underlayer film formation for lithography.

[28]

The composition according to the above [26], which is used in resist film formation.

[29]

The composition according to the above [26], which is used in resist permanent film formation.

[30]

The composition according to any one of the above [21] to [25], which is used in optical component formation.

[31]

A resist pattern formation method, comprising: an underlayer film formation step of forming an underlayer film on a substrate using the composition according to any one of the above [21] to [25]; a photoresist film formation step of forming at least one photoresist film on the underlayer film formed through the underlayer film formation step; and a step of irradiating a predetermined region of the photoresist film formed through the photoresist film formation step with radiation for development.

[32]

A resist pattern formation method, comprising:
a photoresist film formation step of forming a photoresist film on a substrate using the composition according to any one of the above [21] to [25]; and
a development step of irradiating a predetermined region of the photoresist film formed through the photoresist film formation step with radiation for development.
   [33]

A circuit pattern formation method, comprising:
an underlayer film formation step of forming an underlayer film on a substrate using the composition according to any one of the above [21] to [25];
an intermediate layer film formation step of forming an intermediate layer film on the underlayer film formed through the underlayer film formation step;
a photoresist film formation step of forming at least one photoresist film on the intermediate layer film formed through the intermediate layer film formation step;
a resist pattern formation step of irradiating a predetermined region of the photoresist film formed through the photoresist film formation step with radiation for development to form a resist pattern;
an intermediate layer film pattern formation step of etching the intermediate layer film with the resist pattern formed through the resist pattern formation step as a mask to form an intermediate layer film pattern;
an underlayer film pattern formation step of etching the underlayer film with the intermediate layer film pattern formed through the intermediate layer film pattern formation step as a mask to form an underlayer film pattern; and
a substrate pattern formation step of etching the substrate with the underlayer film pattern formed through the underlayer film pattern formation step as a mask to form a pattern on the substrate.
   [34]

A method for purifying the compound according to any one of the above [1] to [17] or the resin according to any one of the above [18] to [20], comprising:
an extraction step of bringing a solution containing the compound or the resin and an organic solvent that does not inadvertently mix with water into contact with an acidic aqueous solution to carry out extraction.

### Advantageous Effects of Invention

According to the present invention, a new compound that is useful as a film forming material for lithography or an optical component forming material, a resin having a constituent unit derived from said compound, a composition, a resist pattern formation method, a circuit pattern formation method, and a purification method can be provided.

### DESCRIPTION OF EMBODIMENTS

Hereinafter, an embodiment of the present invention will be described (hereinafter, referred to as the “present embodiment”). The embodiments described below are given merely for illustrating the present invention.

The present invention is not limited only by these embodiments.

### [Compound (1)]

A compound of the present embodiment is a compound represented by the following formula (1) (hereinafter, also simply referred to as “compound (1)”).

Compound (1) of the present embodiment has, for example, the following characteristics (I) to (IV).
(I) Compound (1) of the present embodiment has excellent solubility in an organic solvent (particularly, a safe solvent). Therefore, for example, when compound (1) of the present embodiment is used as a film forming material for lithography, films for lithography can be formed by a wet process such as spin coating or screen printing.
(II) In compound (1) of the present embodiment, the carbon concentration is relatively high and the oxygen concentration is relatively low. In addition, since the compound of the present embodiment has a phenolic hydroxy group in the molecule, it is useful for formation of a cured product through the reaction with a curing agent, but it can also form a cured product on its own through the crosslinking reaction of the phenolic hydroxy group upon baking at a high temperature. Due to the above, compound (1) of the present embodiment can exhibit high heat resistance, and when compound (1) of the present embodiment is used as a film forming material for lithography, degradation of the film upon baking at a high temperature is suppressed and a film for lithography excellent in etching resistance to oxygen plasma etching and the like can be formed.
(III) Compound (1) of the present embodiment can exhibit high heat resistance and etching resistance, as described above, and also has excellent adhesiveness to a resist film and a resist intermediate layer film material. Therefore, when compound (1) of the present embodiment is used as a film forming material for lithography, films for lithography excellent in resist pattern formability can be formed. The term “resist pattern formability” herein refers to a property in which there are no major defects in the resist pattern shape and both resolution and sensitivity are excellent.
(IV) Compound (1) of the present embodiment has a high refractive index due to its high aromatic ring density, and even after heat treatment, coloration is suppressed and transparency is excellent.

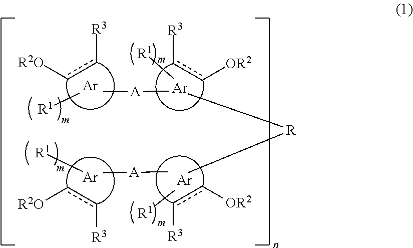

In formula (1),
each A is independently a single bond or a linking group;
Ar is an aromatic ring;
R is a 2n-valent group having 1 to 60 carbon atoms and optionally having a substituent and/or a heteroatom;
each R¹ is independently a linear, branched, or cyclic alkyl group having 1 to 30 carbon atoms, an aryl group having 6 to 40 carbon atoms, an alkenyl group having 2 to 30 carbon atoms, an alkynyl group having 2 to 30 carbon atoms, a halogen atom, a nitro group, an amino group, a carboxy group, a cyano group, a mercapto group, or a hydroxy group;
each R² is independently a hydrogen atom, a crosslinkable group, a dissociable group, a linear, branched, or cyclic alkyl group having 1 to 30 carbon atoms, or an aryl group having 6 to 40 carbon atoms;
provided that at least one R² is any of a hydrogen atom, a crosslinkable group, and a dissociable group;
each R³ is independently a linear, branched, or cyclic alkyl group having 1 to 30 carbon atoms, an aryl group having 6 to 40 carbon atoms, an alkenyl group having 2 to 30 carbon atoms, an alkynyl group having 2 to 30 carbon atoms, a halogen atom, a nitro group, an amino group, a carboxy group, a cyano group, a mercapto group, or a hydroxy group;
each m is independently an integer of 0 to 8;
n is an integer of 1 to 4;
the alkyl group, the aryl group, the alkenyl group, and the alkynyl group optionally have a substituent and/or a heteroatom.

Compound (1) of the present embodiment is preferably a compound represented by the following formula (1-1).

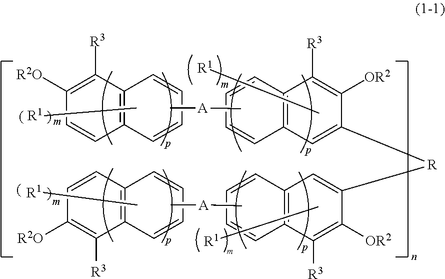

In formula (1-1),
A, R, R¹ to R³, n, and m are each as defined in the above formula (1), and
each p is independently an integer of 0 to 3.

The compound represented by the following formula (A) may be excluded from compound (1) of the present embodiment.

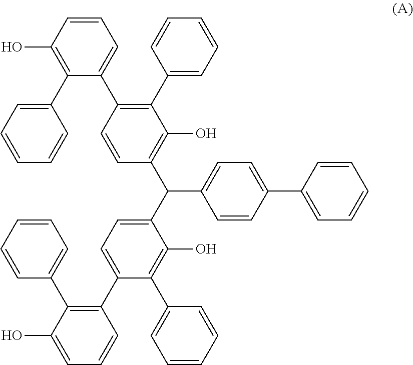 [SMILES: Oc1cccc(-c2ccc(C(c3ccc(-c4ccccc4)cc3)c3ccc(-c4cccc(O)c4-c4ccccc4)c(-c4ccccc4)c3O)c(O)c2-c2ccccc2)c1-c1ccccc1]

Hereinafter, examples of the substituent for each group include, but not particularly limited to, a halogen atom, an alkyl group, an aryl group, an aralkyl group, an alkenyl group, an acyl group, an alkoxycarbonyl group, an alkyloyloxy group, an aryloyloxy group, a cyano group, and a nitro group.

Examples of the halogen atom include, but not particularly limited to, a chlorine atom, a bromine atom, and an iodine atom.

The alkyl group may be linear, branched, or cyclic. Examples of the alkyl group include, but not particularly limited to, an alkyl group having 1 to 10 carbon atoms such as a methyl group, a tert-butyl group, a cyclohexyl group, and an adamantyl group.

Examples of the aryl group include, but not particularly limited to, an aryl group having 6 to 203 carbon atoms such as a phenyl group, a tolyl group, and a naphthyl group. The aryl group may further have a substituent such as a halogen atom and an alkyl group having 1 to 5 carbon atoms.

Examples of the aralkyl group include, but not particularly limited to, a benzyl group. The aralkyl group may further have a substituent such as a halogen atom and an alkyl group having 1 to 5 carbon atoms.

Examples of the acyl group include, but not particularly limited to, an aliphatic acyl group having 1 to 6 carbon atoms such as a formyl group and an acetyl group, and an aromatic acyl group such as a benzoyl group.

Examples of the alkoxycarbonyl group include, but not particularly limited to, an alkoxycarbonyl group having 2 to 5 carbon atoms such as a methoxycarbonyl group.

Examples of the alkyloyloxy group include, but not particularly limited to, an acetoxy group.

Examples of the aryloxy group include, but not particularly limited to, a benzoyloxy group.

Examples of the heteroatom include, but not particularly limited to, an oxygen atom, a sulfur atom, a selenium atom, a nitrogen atom, and a phosphorus atom.

A carbon atom of each group may be substituted with the heteroatom.

In the case of including the above-described substituents, the number of carbon atoms in each group described in the present specification is the total number of carbon atoms including the substituents.

### <Crosslinkable Group>

The “crosslinkable group” is a group that crosslinks in the presence of a catalyst or without a catalyst. Examples of the crosslinkable group include, but not particularly limited to, a group having a hydroxy group, a group having an epoxy group, a group having a carbon-carbon double bond, and a group having a carbon-carbon triple bond.

Examples of the group having a carbon-carbon double bond include, but not particularly limited to, a group having an allyl group, a (meth)acryloyl group, an epoxy (meth)acryloyl group, a group having a urethane (meth)acryloyl group, and a group having a vinylphenyl group.

The group having a carbon-carbon triple bond includes a group having an alkynyl group.

Examples of the group having a carbon-carbon double bond include a group represented by the following formula (X).

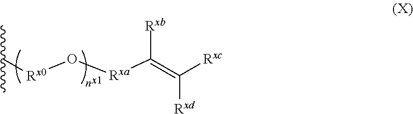

In formula (X), R^{x0} is an ethylene group or a propylene group; n^{x1} is 0 to 5; R^{xa} is a single bond or a linking group; R^{xb}, R^{xc}, and R^{xd} are each independently a hydrogen atom or a methyl group. Preferably, n^{x1} is 1 to 5.

Examples of the group having an allyl group include, but not particularly limited to, a group represented by the following formula (X-1).

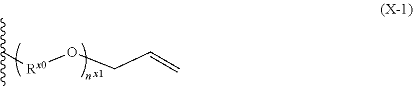

In formula (X-1), R^{x0} is an ethylene group or a propylene group; and n^{x1} is 0 to 5. Preferably, n^{x1} is 1 to 5.

Examples of the group having a (meth)acryloyl group include, but not particularly limited to, a group represented by the following formula (X-2).

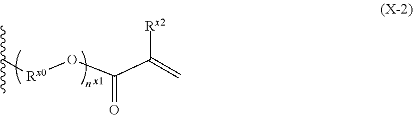

In formula (X-2), R^{x0} and n^{x1} are each as defined in formula (X-1); and R^{x2} is a hydrogen atom or a methyl group.

Examples of the group having an epoxy (meth)acryloyl group include, but not particularly limited to, a group represented by the following formula (X-3). Here, the epoxy (meth)acryloyl group refers to a group generated through a reaction between an epoxy (meth)acrylate and a hydroxy group.

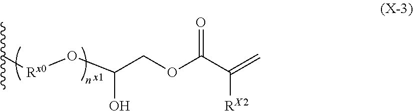

In formula (X-3), R^{x0} and n^{x1} are as defined in formula (X-1); and R^{x2} has the same definition as in formula (X-2).

Examples of the group having a urethane (meth)acryloyl group include, but not particularly limited to, a group represented by the following formula (X-4).

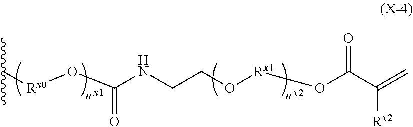

In formula (X-4), R^{X0} and n^{x1} are each as defined in formula (X-1); R^{x2} has the same definition as in formula (X-2); R^{x1} is an ethylene group or a propylene group; and n^{x2} is 0 to 5. Preferably, n^{x2} is 1 to 5.

Examples of the group having a vinylphenyl group include, but not particularly limited to, a group represented by the following formula (X-5).

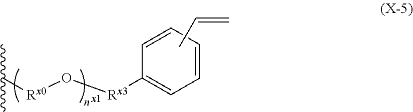

In formula (X-5), R^{x0} and n^{x1} are each as defined in formula (X-1); and R^{x2} is a single bond or a divalent aliphatic hydrocarbon group having 1 to 3 carbon atoms. The divalent aliphatic hydrocarbon group includes a methylene group and an ethylene group.

Examples of the group having a hydroxy group include a group represented by the following formula (Y1).

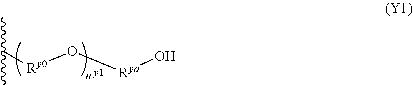

In formula (Y1), R^{y0} is an ethylene group or a propylene group; n^{y1} is 0 to 5; and R^{ya} is a divalent aliphatic hydrocarbon group having 1 to 3 carbon atoms. Preferably, n^{y1} is 1 to 5.

The group having a hydroxy group is preferably a group represented by the following formula (Y1-1) or formula (Y1-2).

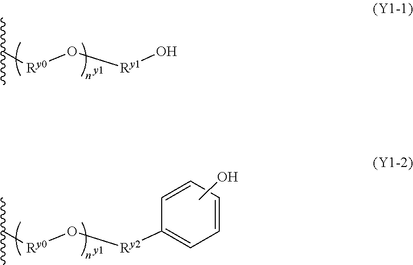

In formula (Y1-1) and formula (Y1-2), R^{y0} and n^{y1} are each as defined in formula (Y1); R^{y1} is an ethylene group or a propylene group; and R^{y2} is a single bond or a divalent aliphatic hydrocarbon group having 1 to 3 carbon atoms. The divalent aliphatic hydrocarbon group includes a methylene group and an ethylene group.

Examples of the group having a glycidyl group include, but not particularly limited to, a group represented by the following formula (Y2).

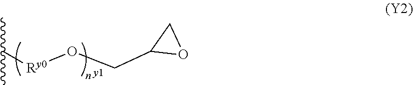

In formula (Y2), R^{y0} and n^{y1} are each as defined in formula (Y1).

Examples of the group having a carbon-carbon triple bond include a group represented by the following formula (Z).

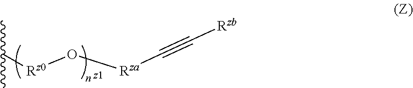

In formula (Z), R^{z0} is an ethylene group or a propylene group; n^{z1} is 0 to 5; R^{za} is a single bond or a linking group; R^{zb} is a hydrogen atom or a monovalent hydrocarbon group having 1 to 20 carbon atoms. Preferably, n^{y1} is 1 to 5.

Examples of the group having a carbon-carbon triple bond include a substituted or unsubstituted ethynyl group, and a group represented by the following formula (Z-1), formula (Z-2), formula (Z-3), or formula (Z-4).

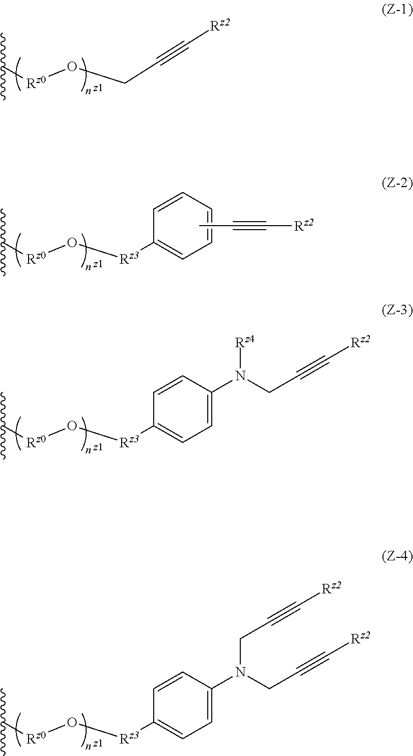

In formula (Z-1), formula (Z-2), formula (Z-3), or formula (Z-4), R^{z0} and n^{z1} are each as defined in formula (Z); and R^{z3} is a single bond or a divalent aliphatic hydrocarbon group having 1 to 3 carbon atoms. The divalent aliphatic hydrocarbon group includes a methylene group and an ethylene group.

R^{z2} and R^{z4} are each independently a hydrogen atom or a monovalent hydrocarbon group having 1 to 20 carbon atoms.

Among the above, from the viewpoint of ultraviolet curability, as the crosslinkable group, a (meth)acryloyl group, an epoxy (meth)acryloyl group, a urethane (meth)acryloyl group, a group having a glycidyl group, and a group containing a styrene group are preferable, a (meth)acryloyl group, an epoxy (meth)acryloyl group, and a group having a urethane (meth)acryloyl group are more preferable, and a group having a (meth)acryloyl group is still more preferable.

### <Dissociable Group>

The “dissociable group” means a group that is dissociated in the presence of a catalyst or without a catalyst. In addition, the “acid dissociable group” means a group that is cleaved in the presence of an acid to generate an alkali soluble group. Examples of the alkali soluble group include, but not particularly limited to, a phenolic hydroxy group, a carboxy group, a sulfonic acid group, and a hexafluoroisopropanol group. Among them, from the viewpoint of ease in obtaining introducing reagents, a phenolic hydroxy group and a carboxy group are preferable, and a phenolic hydroxy group is particularly preferable. The acid dissociable group preferably has the property of causing chained cleavage reaction in the presence of an acid, for achieving pattern formation with high sensitivity and high resolution. While the acid dissociable group is not particularly limited, the acid dissociable group can be used by appropriately selecting from hydroxystyrene resins used for chemical amplification resist compositions for KrF and ArF and acid dissociable groups proposed for (meth)acrylic resins and the like, for example.

Specific examples of the acid dissociable group include, but not particularly limited to, a substituted methyl group, a 1-substituted ethyl group, a 1-substituted n-propyl group, a 1-branched alkyl group, a silyl group, an acyl group, a 1-substituted alkoxymethyl group, a cyclic ether group, an alkoxycarbonyl group, an alkoxycarbonylalkyl group, and the like. The acid dissociable group preferably has no crosslinkable functional group.

Preferable examples of the acid dissociable group include a group selected from the group consisting of a 1-substituted ethyl group, a 1-substituted n-propyl group, a 1-branched alkyl group, a silyl group, an acyl group, a 1-substituted alkoxymethyl group, a cyclic ether group, and a group having an alkoxycarbonyl group.

The acid dissociable group is represented by the following formula (A), for example.

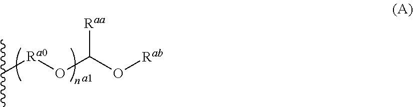

In formula (A), R^{a0} is an ethylene group or a propylene group; n^{a1} is 0 to 5; R^{aa} is a hydrogen atom or a linear, branched, or cyclic alkyl group having 1 to 30 carbon atoms; and R^{ab} is a linear, branched, or cyclic alkyl group having 1 to 30 carbon atoms.

The substituted methyl group is preferably a substituted methyl group having 2 to 20 carbon atoms, more preferably a substituted methyl group having 4 to 18 carbon atoms, and still more preferably a substituted methyl group having 6 to 16 carbon atoms. Examples of the substituted methyl group can include, but not particularly limited to, a methoxymethyl group, a methylthiomethyl group, an ethoxymethyl group, a n-propoxymethyl group, an isopropoxymethyl group, a n-butoxymethyl group, a t-butoxymethyl group, a 2-methylpropoxymethyl group, an ethylthiomethyl group, a methoxyethoxymethyl group, a phenyloxymethyl group, a 1-cyclopentyloxymethyl group, a 1-cyclohexyloxymethyl group, a benzylthiomethyl group, a phenacyl group, a 4-bromophenacyl group, a 4-methoxyphenacyl group, a piperonyl group, and a group represented by the following formula (A-1).

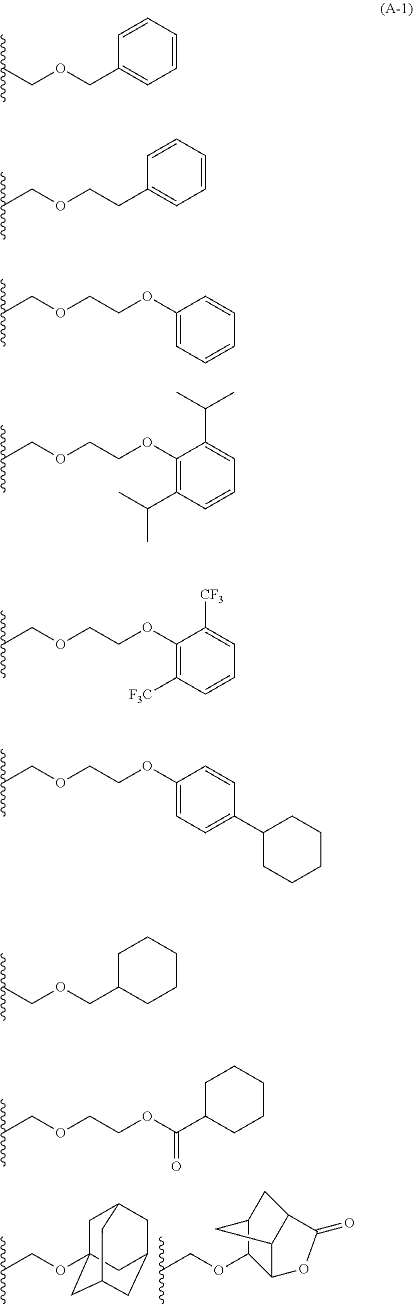

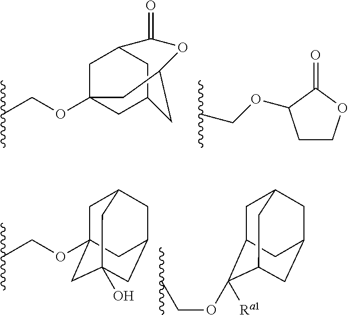

(In formula (A-1), R^{a1} is an alkyl group having 1 to 4 carbon atoms.)

The 1-substituted ethyl group is preferably a 1-substituted ethyl group having 3 to 20 carbon atoms, more preferably a 1-substituted ethyl group having 5 to 18 carbon atoms, and still more preferably a substituted ethyl group having 7 to 16 carbon atoms. Examples of the 1-substituted ethyl group can include, but not particularly limited to, a 1-methoxyethyl group, 1-methylthioethyl group, a 1,1-dimethoxyethyl group, a 1-ethoxyethyl group, a 1-ethylthioethyl group, a 1,1-diethoxyethyl group, a 1-n-propoxyethyl group, a 1-isopropoxyethyl group, a 1-n-butoxyethyl group, a 1-t-butoxyethyl group, a 1-phenoxyethyl group, a 1-phenylthioethyl group, a 1,1-diphenoxyethyl group, a 1-cyclopentyloxyethyl group, a 1-cyclohexyloxyethyl group, a 1-phenylethyl group, a 1,1-diphenylethyl group, a group represented by the following formula (A-2), and the like.

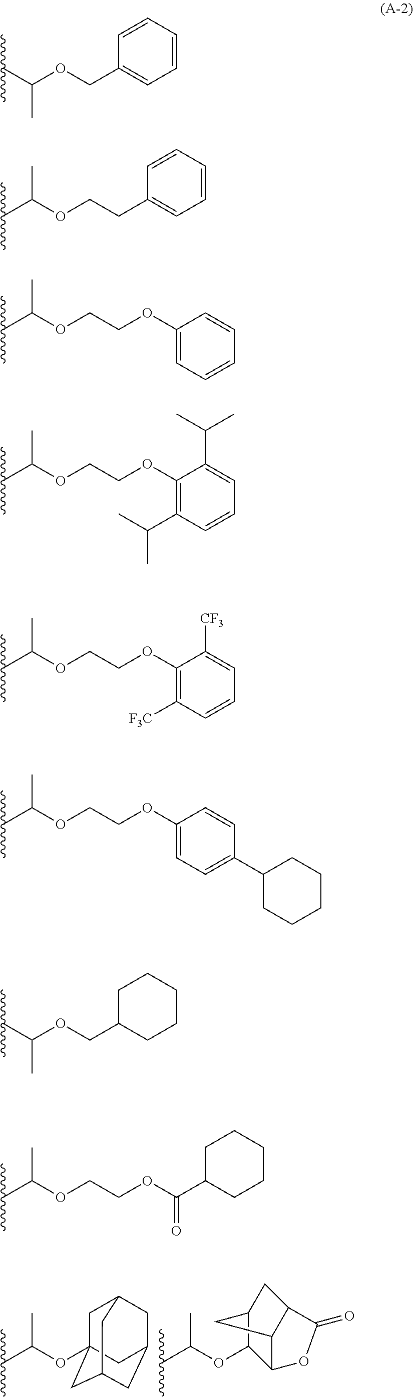

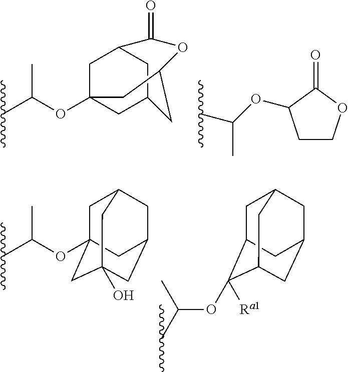

(In formula (A-2), R^{a1} has the same definition as in the above formula (A-2).)

The 1-substituted n-propyl group is preferably a 1-substituted n-propyl group having 4 to 20 carbon atoms, more preferably a 1-substituted n-propyl group having 6 to 18 carbon atoms, and still more preferably a 1-substituted n-propyl group having 8 to 16 carbon atoms. Examples of the 1-substituted n-propyl group can include, but not particularly limited to, a 1-methoxy-n-propyl group and 1-ethoxy-n-propyl group, a 1-propoxy-n-propyl group, and the like.

The 1-branched alkyl group is preferably a 1-branched alkyl group having 3 to 20 carbon atoms, more preferably a 1-branched alkyl group having 5 to 18 carbon atoms, and still more preferably a 1-branched alkyl group having 7 to 16 carbon atoms. Examples of the 1-branched alkyl group can include, but not particularly limited to, an isopropyl group, a sec-butyl group, a tert-butyl group, a 1,1-dimethylpropyl group, a 1-methylbutyl group, a 1,1-dimethylbutyl group, a 2-methyladamantyl group, and a 2-ethyladamantyl group.

The silyl group is preferably a silyl group having 1 to 20 carbon atoms, more preferably a silyl group having 3 to 18 carbon atoms, and still more preferably a silyl group having 5 to 16 carbon atoms. Examples of the silyl group can include, but not particularly limited to, a trimethylsilyl group, an ethyldimethylsilyl group, a methyldiethylsilyl group, a triethylsilyl group, a tert-butyldimethylsilyl group, a tert-butyldiethylsilyl group, a tert-butyldiphenylsilyl group, a tri-tert-butylsilyl group, and a triphenylsilyl group.

The acyl group is preferably an acyl group having 2 to 20 carbon atoms, more preferably an acyl group having 4 to 18 carbon atoms, and still more preferably an acyl group having 6 to 16 carbon atoms. Examples of the acyl group can include, but not particularly limited to, an acetyl group, a phenoxyacetyl group, a propionyl group, a butyryl group, a heptanoyl group, a hexanoyl group, a valeryl group, a pivaloyl group, an isovaleryl group, a lauroyl group, an adamantylcarbonyl group, a benzoyl group, and a naphthoyl group.

The 1-substituted alkoxymethyl group is preferably a 1-substituted alkoxymethyl group having 2 to 20 carbon atoms, more preferably a 1-substituted alkoxymethyl group having 4 to 18 carbon atoms, and still more preferably a 1-substituted alkoxymethyl group having 6 to 16 carbon atoms. Examples of the 1-substituted alkoxymethyl group can include, but not particularly limited to, a 1-cyclopentylmethoxymnethyl group, a 1-cyclopentylethoxymethyl group, a 1-cyclohexylmethoxymethyl group, a 1-cyclohexylethoxymethyl group, a 1-cyclooctylmethoxymethyl group, and a 1-adamantylmethoxymethyl group.

The cyclic ether group is preferably a cyclic ether group having 2 to 20 carbon atoms, more preferably a cyclic ether group having 4 to 18 carbon atoms, and still more preferably a cyclic ether group having 6 to 16 carbon atoms. Examples of the cyclic ether group can include, but not particularly limited to, a tetrahydropyranyl group, a tetrahydrofuranyl group, a tetrahydrothiopyranyl group, a tetrahydrothiofuranyl group, a 4-methoxytetrahydropyranyl group, and a 4-methoxytetrahydrothiopyranyl group.

The group having an alkoxycarbonyl group is represented by the following formula (B), for example.

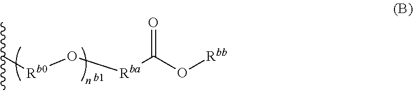

In formula (B), R^{b0} is an ethylene group or a propylene group; n^{b1} is 0 to 5; R^{ba} is a single bond or a linking group; and R^{bb} is a linear, branched, or cyclic alkyl group having 1 to 30 carbon atoms.

Examples of the group having an alkoxycarbonyl group include, but not particularly limited to, an alkoxycarbonyl group and an alkoxycarbonylalkyl group.

The alkoxycarbonyl group is preferably an alkoxycarbonyl group having 2 to 20 carbon atoms, more preferably an alkoxycarbonyl group having 4 to 18 carbon atoms, and still more preferably an alkoxycarbonyl group having 6 to 16 carbon atoms. Examples of the alkoxycarbonyl group can include, but not particularly limited to, a methoxycarbonyl group, an ethoxycarbonyl group, a n-propoxycarbonyl group, an isopropoxycarbonyl group, a n-butoxycarbonyl group, a tert-butoxycarbonyl group, and a group represented by the following formula (B-1) wherein n=0.

The alkoxycarbonylalkyl group is preferably an alkoxycarbonylalkyl group having 2 to 20 carbon atoms, more preferably an alkoxycarbonylalkyl group having 4 to 18 carbon atoms, and still more preferably an alkoxycarbonylalkyl group having 6 to 16 carbon atoms. Examples of the alkoxycarbonylalkyl group can include, but not particularly limited to, a methoxycarbonylmethyl group, an ethoxycarbonylmethyl group, a n-propoxycarbonylmethyl group, an isopropoxycarbonylmethyl group, a n-butoxycarbonylmethyl group, and a group represented by the following formula (B-1) wherein n=1 to 4.

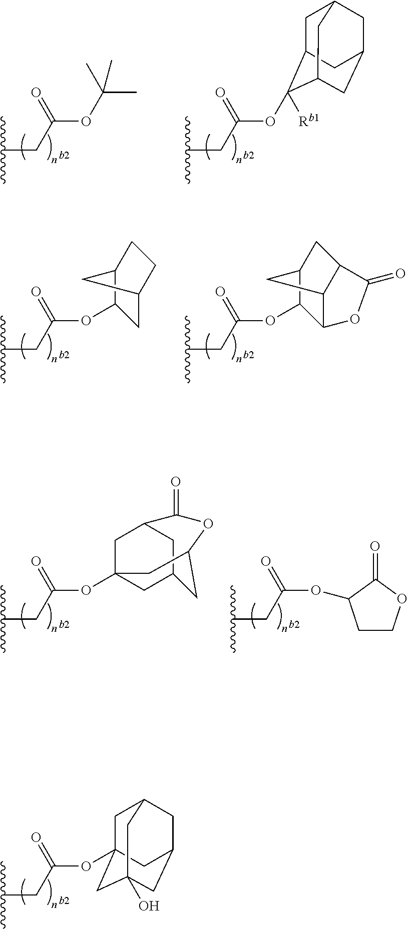

(In formula (B-1), R^{b1} is a hydrogen atom or a linear or branched alkyl group having 1 to 4 carbon atoms; and n^{b2} is an integer of 0 to 4.)

Among these acid dissociable groups, a substituted methyl group, a 1-substituted ethyl group, a 1-substituted alkoxymethyl group, a cyclic ether group, an alkoxycarbonyl group, and an alkoxycarbonylalkyl group are preferable; a substituted methyl group, a 1-substituted ethyl group, an alkoxycarbonyl group, and an alkoxycarbonylalkyl group are more preferable because of their high sensitivity; and an acid dissociable group having a structure selected from a cycloalkane having 3 to 12 carbon atoms, a lactone, and an aromatic ring having 6 to 12 carbon atoms is further preferable. The cycloalkane having 3 to 12 carbon atoms may be monocyclic or polycyclic but is preferably polycyclic. Specific examples thereof include, but not particularly limited to, a monocycloalkane, a bicycloalkane, a tricycloalkane, and a tetracycloalkane. More specific examples thereof include, but not limited to, a monocycloalkane such as cyclopropane, cyclobutane, cyclopentane, and cyclohexane; and a polycycloalkane such as adamantane, norbornane, isobornane, tricyclodecane, and tetracyclodecane. Among them, adamantane, tricyclodecane, and tetracyclodecane are preferable, and adamantane and tricyclodecane are especially preferable. The cycloalkane having 3 to 12 carbon atoms optionally has a substituent. Examples of the lactone include, but not particularly limited to, a cycloalkane group having 3 to 12 carbon atoms and having a butyrolactone or lactone group. Examples of the aromatic ring having 6 to 12 carbon atoms include, but not particularly limited to, a benzene ring, a naphthalene ring, an anthracene ring, a phenanthrene ring, and a pyrene ring. A benzene ring and a naphthalene ring are preferable, and a naphthalene ring is especially preferable.

A group selected from the group consisting of groups represented by the following formula (B-2) has high resolution and therefore is especially preferable.

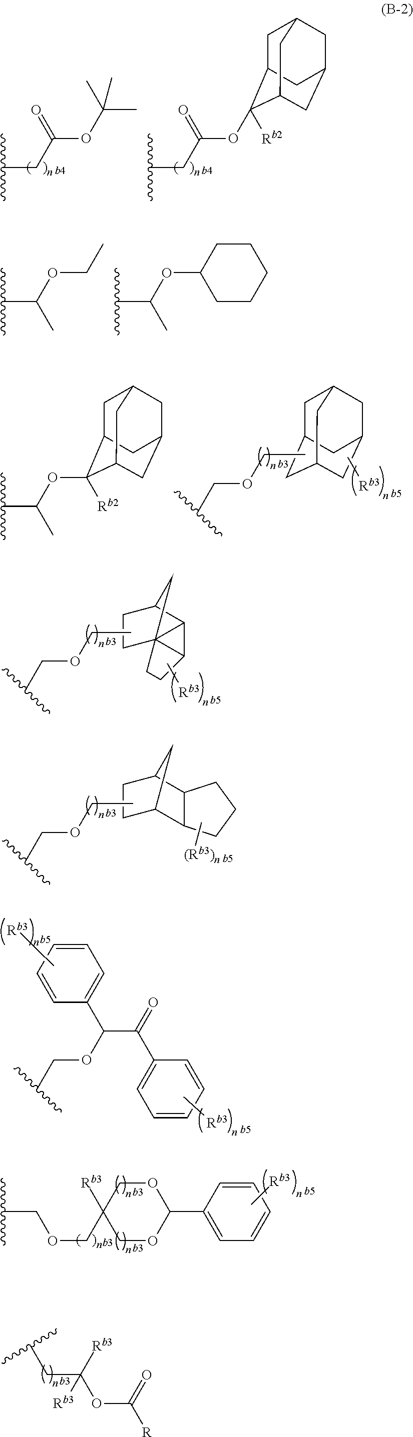

(In formula (B-2), R^{b2} is a hydrogen atom or a linear or branched alkyl group having 1 to 4 carbon atoms; R^{b3} is a hydrogen atom, a linear or branched alkyl group having 1 to 4 carbon atoms, a cyano group, a nitro group, a heterocyclic group, a halogen atom, or a carboxy group; n^{b5} is an integer of 0 to 4; n^{b6} is an integer of 1 to 5; and n^{b4} is an integer of 0 to 4.)

In formula (1),

A is a single bond or a linking group.

When p=0, a substitution position of A may be any of a ortho position, meta position, and para position but is preferably a para position with respect to the R²O— group.

A is preferably a single bond from the viewpoint of enhancing heat resistance.

A is preferably a linking group from the viewpoint of enhancing flatness.

Examples of the linking group as A include, but not particularly limited to, a carbonyl group (>C═O group), a thiocarbonyl group (>C═S group), a divalent hydrocarbon group having 1 to 12 carbon atoms, a divalent heteroatom, a —SO— group, and a —SO₂— group.

The divalent hydrocarbon group may be linear, branched, or cyclic.

Examples of the divalent hydrocarbon group include, but not particularly limited to, a methylene group; an ethylene group such as an ethane-1,2-diyl group and an ethane-1,1-diyl group; a propylene group such as a propane-1,3-diyl group, a propane-2,2-diyl group, and a propane-1,1-diyl group; a butylene group such as a butane-2,2-diyl group; a hexafluoropropylene group such as a 1,1,1,3,3,3-hexafluoropropane-2,2-diyl group; a vinylidene chloride-2,2-diyl group; a phenylethylene group; a diphenylmethylene group; a cyclohexylene group; a 3,3,5-trimethylcyclohexane-1,1-diyl group; a trimethylcyclohexylene group; a cyclododecylene group, and a group represented by the following formula (C).

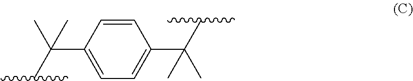 [SMILES: *C(C)(C)c1ccc(C(*)(C)C)cc1]

The divalent hydrocarbon group optionally has a substituent and/or a heteroatom.

Among these divalent hydrocarbon groups, from the viewpoint of solubility, a cyclic divalent hydrocarbon group is preferable, and a cyclohexylene group, a trimethylcyclohexylene group, and a cyclododecylene group are more preferable.

Among these divalent hydrocarbon groups, from the viewpoint of solubility, A is preferably a hydrocarbon group having a halogen atom and more preferably a hexafluoropropylene group.

In addition, among these divalent hydrocarbon groups, from the viewpoint of further improving resist pattern formability, A is preferably a single bond or a linear or branched hydrocarbon group, and more preferably a single bond, a methylene group, or a 2,2-propanediyl group.

The divalent heteroatom includes a divalent oxygen atom (—O—) and a divalent a sulfur atom (—S—).

In formula (1), Ar is an aromatic ring. Ar means a moiety represented by the following formula.

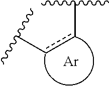

The double bond in the above formula means carbon atoms having an sp² hybrid orbital forming an aromatic ring and means that the adjacent carbon atom has a substituent.

Examples of the aromatic ring as Ar include, but not particularly limited to, benzene, naphthalene, anthracene, phenanthrene, tetracene, chrysene, triphenylene, pyrene, pentacene, benzopyrene, coronene, azulene, and fluorene. Among them, benzene, naphthalene, and anthracene are preferable, and benzene and naphthalene are more preferable.

In addition, the aromatic ring as Ar is preferably an aromatic ring represented by the following formula (Ar). The aromatic ring represented by formula (Ar) is a structure schematically representing an aromatic ring and includes isomeric structures.

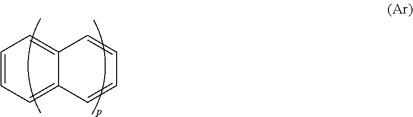

In formula (Ar), p is an integer of 0 to 3.

Examples of the aromatic ring represented by formula (Ar) are as follows.

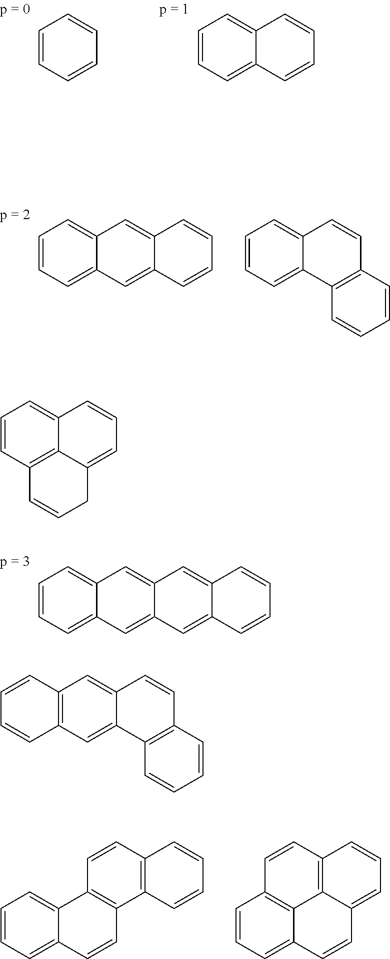

In formula (1), R is a 2n-valent group having 1 to 30 carbon atoms and optionally having a substituent and/or a heteroatom, and each aromatic ring is bonded via this R. Specific examples of the 2n-valent group will be mentioned later.

In formula (1), each R¹ is independently a linear, branched, or cyclic alkyl group having 1 to 30 carbon atoms, an aryl group having 6 to 40 carbon atoms, an alkenyl group having 2 to 30 carbon atoms, an alkynyl group having 2 to 30 carbon atoms, a halogen atom, a nitro group, an amino group, a carboxy group, a cyano group, a mercapto group, or a hydroxy group.

Examples of the above alkyl group include, but not particularly limited to, a linear or branched alkyl group such as a methyl group, an ethyl group, a n-propyl group, an isopropyl group, a n-butyl group, an isobutyl group, a sec-butyl group, a tert-butyl group, a pentyl group, and a hexyl group; and a cyclic alkyl group such as a cyclopentyl group and a cyclohexyl group.

Examples of the above aryl group include, but not particularly limited to, a phenyl group, a naphthyl group, a tolyl group, and a xylyl group.

Examples of the above alkenyl group include, but not particularly limited to, an ethenyl group, a propenyl group, a butenyl group, a pentenyl group, and a hexenyl group.

Examples of the above alkynyl group include, but not particularly limited to, an ethynyl group, a propynyl group, a butynyl group, a pentynyl group, and a hexynyl group.

Examples of the above halogen atom include, but not particularly limited to, fluorine, chlorine, bromine, and iodine.

Among them, R¹ is preferably a linear, branched, or cyclic alkyl group having 1 to 30 carbon atoms or an aryl group having 6 to 40 carbon atoms, more preferably a methyl group or a phenyl group, and still more preferably a methyl group.

In formula (1), each m is independently an integer of 0 to 8, preferably an integer of 0 to 2, more preferably an integer of 0 or 1, and still more preferably 0.

In formula (1), each R² is independently a hydrogen atom, a crosslinkable group, a dissociable group, a linear, branched, or cyclic alkyl group having 1 to 30 carbon atoms, or an aryl group having 6 to 40 carbon atoms. The linear, branched, or cyclic alkyl group having 1 to 30 carbon atoms and the aryl group having 6 to 40 carbon atoms are preferably groups different from the crosslinkable group and the dissociable group. Examples of the above alkyl group and aryl group are the same groups as in R¹ described above.

Provided that, from the viewpoints of facilitating crosslinking reaction and solubility in an organic solvent, at least one R² is any of a hydrogen atom, a crosslinkable group, and a dissociable group and is preferably a hydrogen atom.

The crosslinkable group is preferably a group having a hydroxy group, a group having an epoxy group, a group having a carbon-carbon double bond, or a group having a carbon-carbon triple bond and is more preferably a group represented by formula (X), formula (Y1), formula (Y2), or formula (Z).

The dissociable group is preferably an alkoxycarbonyl group or an alkoxycarbonylalkyl group and is more preferably a tert-butoxycarbonyl group or a group represented by the following formula (B-3).

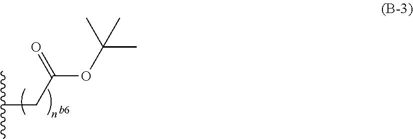

In formula (B-3), n^{b6} is an integer of 0 to 3.

The number of R²s, which are each any of a hydrogen atom, a crosslinkable group, and a dissociable group, is preferably two or more, more preferably three or more, and still more preferably four or more from the viewpoints of facilitating crosslinking reaction and solubility in an organic solvent.

In formula (1), each R³ is independently a linear, branched, or cyclic alkyl group having 1 to 30 carbon atoms, an aryl group having 6 to 40 carbon atoms, an alkenyl group having 2 to 30 carbon atoms, an alkynyl group having 2 to 30 carbon atoms, a halogen atom, a nitro group, an amino group, a carboxy group, a cyano group, a mercapto group, or a hydroxy group.

Examples of the above alkyl group, aryl group, alkenyl group, alkynyl group, and halogen atom are the same groups as in R¹ described above.

R³ is preferably a linear, branched, or cyclic alkyl group having 1 to 30 carbon atoms, an aryl group having 6 to 40 carbon atoms, an alkenyl group having 2 to 30 carbon atoms, an alkynyl group having 2 to 30 carbon atoms, a halogen atom, a nitro group, an amino group, a carboxy group, a cyano group, a mercapto group, or a hydroxy group; more preferably a linear, branched, or cyclic alkyl group having 1 to 30 carbon atoms or an aryl group having 6 to 40 carbon atoms; still more preferably a linear or branched alkyl group having 1 to 30 carbon atoms; further preferably a linear or branched alkyl group having 1 to 4 carbon atoms (for example, a methyl group, an ethyl group, a n-propyl group, an isopropyl group, a n-butyl group, a sec-butyl group, or a tert-butyl group) or a phenyl group; and especially preferably a methyl group.

The symbol n is an integer of 1 to 4, preferably an integer of 1 or 2, and more preferably 1.

Each p in formula (1-1) is independently an integer of 0 to 3, preferably an integer of 0 or 1, and more preferably 0.

Examples of the 2n-valent group as R include, but not particularly limited to, a 2n-valent group having 1 to 30 carbon atoms.

The 2n-valent group optionally has the above-described substituent and/or heteroatom.

Examples of the 2n-valent group R include a divalent hydrocarbon group having 1 to 30 carbon atoms (for example, a linear or branched hydrocarbon group or a cyclic hydrocarbon group, such as an alkylene group) when n is 1; a tetravalent hydrocarbon group having 1 to 30 carbon atoms (for example, a linear or branched hydrocarbon group or a cyclic hydrocarbon group, such as an alkanetetrayl group) when n is 2; a hexavalent hydrocarbon group having 2 to 30 carbon atoms (for example, a linear or branched hydrocarbon group or a cyclic hydrocarbon group, such as an alkanehexayl group) when n is 3; and an octavalent hydrocarbon group having 3 to 30 carbon atoms (for example, a linear or branched hydrocarbon group or a cyclic hydrocarbon group, such as an alkaneoctayl group) when n is 4.

Here, the above cyclic hydrocarbon group optionally has a bridged cyclic hydrocarbon group and/or an aromatic group.

Also, the above 2n-valent group R (for example, a 2n-valent hydrocarbon group) optionally has a double bond or triple bond or optionally has a heteroatom.

From the viewpoint of achieving both reactivity and etching resistance, the 2n-valent group is preferably a group having an aliphatic skeleton in which one or more hydrogen atoms are substituted with a bridged cyclic hydrocarbon group and/or aromatic group and more preferably a methylene group in which one or more hydrogen atoms are substituted with a group including an aromatic group or a divalent or tetravalent group having an ethane skeleton in which one or more hydrogen atoms are substituted with a group including an aromatic group. In addition, a methylene group or a divalent or tetravalent group having an ethane skeleton is also preferable from the viewpoint of solubility.

Compound (1) of the present embodiment has high heat resistance attributed to its rigid structure, in spite of its relatively low molecular weight, and can therefore be used even under high temperature baking conditions. Also, when compound (1) of the present embodiment has tertiary carbon or quaternary carbon in the molecule, crystallization is suppressed, and it is thus suitably used as a film forming material for lithography.

Compound (1) of the present embodiment has high solubility in an organic solvent (particularly, a safe solvent) and is excellent in heat resistance and etching resistance. For this reason, a film forming material for lithography containing the compound represented by the above formula (1) has excellent resist pattern formability. Examples of the above organic solvent include the organic solvents described in [Solvent] exemplified in the section of [Composition], which will be mentioned later.

Compound (1) of the present embodiment has a relatively low molecular weight and low viscosity, and therefore facilitates enhancing film flatness while uniformly and completely filling even the steps of a substrate (particularly having fine space, hole pattern, etc.). As a result, a composition for film formation for lithography including the above compound (1) is excellent in embedding properties and flattening properties. In addition, compound (1) has a relatively high carbon concentration, and can therefore exhibit high etching resistance, as well.

Compound (1) of the present embodiment has a high refractive index ascribable to its high aromatic ring density and is prevented from being stained by heat treatment in a wide range from a low temperature to a high temperature. Therefore, compound (1) of the present embodiment is also useful as various optical component forming materials described later. Compound (1) of the present embodiment preferably has quaternary carbon from the viewpoint of preventing the compound from being oxidatively decomposed and stained and improving heat resistance and solvent solubility.

Compound (1) of the present embodiment is preferably a compound represented by the following formula (1a) (hereinafter, also simply referred to as “compound (1a)”) from the viewpoint of facilitating crosslinking and solubility in an organic solvent.

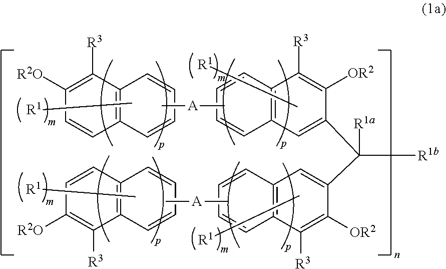

In formula (1a),

A, R¹ to R³, n, m, and p are each as defined in the above formula (1);

R^{1a} is a hydrogen atom or a monovalent group having 1 to 10 carbon atoms;

R^{1b} is an n-valent group having 1 to 30 carbon atoms; and

R^{1a} and R^{1b} may bind to each other to form a cyclic group having 2 to 40 carbon atoms.

The monovalent group and the n-valent group optionally has a substituent and/or a heteroatom.

In formula (1a), R^{1a} is a hydrogen atom or a monovalent group having 1 to 10 carbon atoms.

The monovalent group having 1 to 10 carbon atoms optionally has a substituent and/or a heteroatom.

Examples of the above monovalent group having 1 to 10 carbon atoms include, but not particularly limited to, a linear, branched, or cyclic alkyl group having 1 to 10 carbon atoms, an aryl group having 6 to 10 carbon atoms, an alkenyl group having 2 to 10 carbon atoms, and an alkynyl group having 2 to 10 carbon atoms.

Examples of the above alkyl group include, but not particularly limited to, a linear or branched alkyl group such as a methyl group, an ethyl group, a n-propyl group, an isopropyl group, a n-butyl group, an iso-butyl group, a sec-butyl group, a tert-butyl group, a pentyl group, and a hexyl group; and a cyclic alkyl group such as a cyclopentyl group and a cyclohexyl group.

Examples of the above aryl group include, but not particularly limited to, a phenyl group, a naphthyl group, a tolyl group, and a xylyl group.

Examples of the above alkenyl group include, but not particularly limited to, an ethenyl group, a propenyl group, a butenyl group, a pentenyl group, and a hexenyl group.

Examples of the above alkynyl group include, but not particularly limited to, an ethynyl group, a propynyl group, a butynyl group, a pentynyl group, and a hexynyl group.

Among them, from the viewpoint of solubility, R^{1a} group is preferably a hydrogen atom or a methyl group and more preferably a hydrogen atom.

In formula (1), R^{1b} is an n-valent group having 1 to 30 carbon atoms.

The n-valent group optionally has the above-described substituent and/or heteroatom.

Examples of the n-valent group include a monovalent hydrocarbon group having 1 to 25 carbon atoms (for example, a linear or branched hydrocarbon group or a cyclic hydrocarbon group, such as an alkyl group) when n is 1; a divalent hydrocarbon group having 1 to 25 carbon atoms (for example, a linear or branched hydrocarbon group or a cyclic hydrocarbon group, such as an alkylene group) when n is 2; a trivalent hydrocarbon group having 1 to 25 carbon atoms (for example, a linear or branched hydrocarbon group or a cyclic hydrocarbon group, such as an alkanetriyl group) when n is 3; and an tetravalent hydrocarbon group having 1 to 25 carbon atoms (for example, a linear or branched hydrocarbon group or a cyclic hydrocarbon group, such as an alkanetetrayl group) when n is 4. Here, the above cyclic hydrocarbon group optionally has a bridged cyclic hydrocarbon group and/or an aromatic group.

Among them, from the viewpoint of etching resistance, R^{1b} group is preferably a substituted or unsubstituted phenyl group, a substituted or unsubstituted biphenyl group, or a substituted or unsubstituted naphthyl group; and from the viewpoint of solubility, R^{1b} group is further preferably a substituted or unsubstituted phenyl group or a substituted or unsubstituted biphenyl group. If a substituent present, the substituent is preferably a methyl group, an ethyl group, a propyl group, a butyl group, or a hydroxy group from the viewpoint of solubility. Here, the propyl group and butyl group include isomers.

When R^{1a} and R^{1b} bind to each other to form a cyclic group having 2 to 40 carbon atoms, examples of the cyclic group include a cyclohexane-1,1-diyl group, a fluorene-9,9-diyl group, an acenaphthene-1,1-diyl group, and a 1-acenaphthenon-2,2-diyl group. The number of carbon atoms in the above cyclic group includes carbon to which R^{1a} and R^{1b} bind. In addition, the above examples of the cyclic group are structural examples including carbon to which R^{1a} and R^{1b} bind.

In the compound represented by the above formula (1a), it is preferable that each p is independently an integer of 0 or 1 from the viewpoint of solubility and crosslinkability.

In addition, from the viewpoint of more significantly obtaining the effect of the present invention, the compound represented by the above formula (1a) is preferably a compound represented by the following formula (1b) (hereinafter, also simply referred to as “compound (1b)”) and preferably a compound represented by the following formula (1b′) (hereinafter, also simply referred to as “compound (1b′)”).

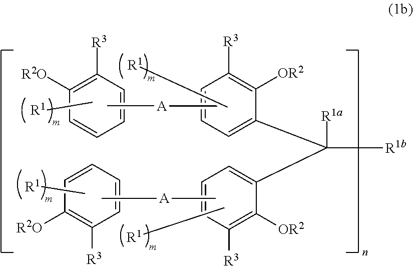

In formula (1b),

A, R¹ to R³, R^{1a}, R^{1b}, n, and m are each as defined in the above formula (1) or the above formula (1a).

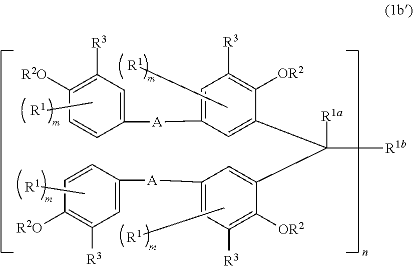

In formula (1b′),

A, R¹ to R³, R^{1a}, R^{1b}, n, and m are each as defined in the above formula (1) or the above formula (1a).

From the viewpoint of more significantly obtaining the effect of the present invention, compound (1b) of the present embodiment is preferably a compound represented by the following formula (1c) (hereinafter, also simply referred to as “compound (1c)”) and preferably a compound represented by the following formula (1c′) (hereinafter, also simply referred to as “compound (1c′)”).

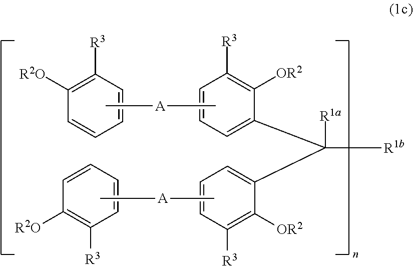

In formula (1c),

A, R², R³, R^{1a}, R^{1b}, and n are each as defined in the above formula (1) or the above formula (1a).

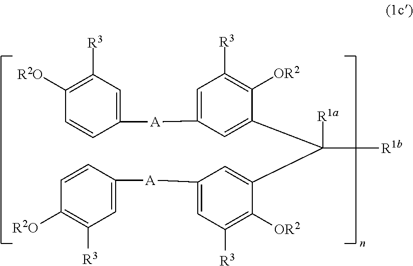

In formula (1c′),

A, R², R³, R^{1a}, R^{1b}, and n are each as defined in the above formula (1) or the above formula (1a).

In compound (1a), compound (1b), and compound (1c) of the present embodiment, R² is preferably a hydrogen atom from the viewpoint of improving solubility and crosslinkability.

In compound (1a), compound (1b), and compound (1c) of the present embodiment, R³ is preferably a methyl group from the viewpoint of flatness, and R³ is preferably a phenyl group from the viewpoint of etching resistance.

In compound (1a), compound (1b), and compound (1c) of the present embodiment, from the viewpoint of improving resist pattern formability, A is preferably a linear or branched hydrocarbon group and more preferably a methylene group or a 2,2-propanediyl group.

In compound (1a), compound (1b), and compound (1c) of the present embodiment, R^{1a} is preferably a hydrogen atom.

In compound (1a), compound (1b), and compound (1c) of the present embodiment, R^{1b} is preferably a phenyl group optionally having a substituent, a biphenyl group optionally having a substituent, or a naphthyl group optionally having a substituent from the viewpoint of etching resistance, and R^{1b} is more preferably a phenyl group optionally having a substituent or a biphenyl group optionally having a substituent from the viewpoint of solubility.

In compound (1a), compound (1b), and compound (1c) of the present embodiment, n is preferably 1.

Compound (1) of the present embodiment is preferably a compound represented by the following formula (1d-1) (hereinafter, also referred to as “compound (1d-1)”) from the viewpoint of more significantly obtaining the effect of the present invention.

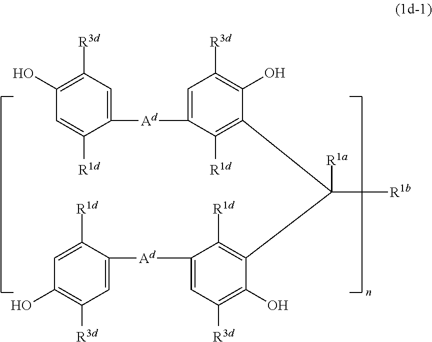

In formula (1d-1),

R^{1a}, R^{1b}, and n are each as defined in the above formula (1) or the above formula (1a); each R^{3d} is independently a linear or branched alkyl group having 1 to 4 carbon atoms or a phenyl group; each R^{1d} is independently a hydrogen atom or a linear or branched alkyl group having 1 to 4 carbon atoms; and A^{d} is a single bond, a methylene group, or a 2,2-propanediyl group. It is preferable that each Rid is independently a methyl group or a phenyl group, and it is more preferable that each R^{3d} is independently a methyl group. It is preferable that each R^{1d} is independently a hydrogen atom or a methyl group, and A^{d} is preferably a single bond.

Compound (1) of the present embodiment is preferably a compound represented by the following formula (1d-1a) (hereinafter, also referred to as “compound (1d-1a)”) from the viewpoint of more significantly obtaining the effect of the present invention.

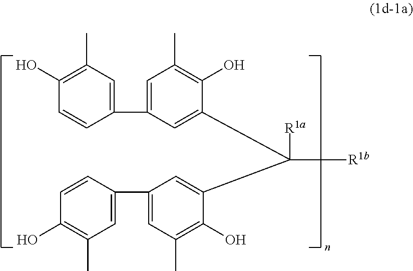

In formula (1d-1a),

R^{1a}, R^{1b}, and n are each as defined in the above formula (1) or the above formula (1a).

In compound (1d-1a) of the present embodiment, R^{1a} is preferably hydrogen from the viewpoint of heat resistance in a nitrogen atmosphere. In addition, R^{1a} is preferably other than hydrogen from the viewpoint of heat resistance in an oxygen atmosphere.

Compound (1) of the present embodiment is preferably a compound represented by the following formula (1d-2) (hereinafter, also referred to as “compound (1d-2)”) from the viewpoint of more significantly obtaining the effect of the present invention.

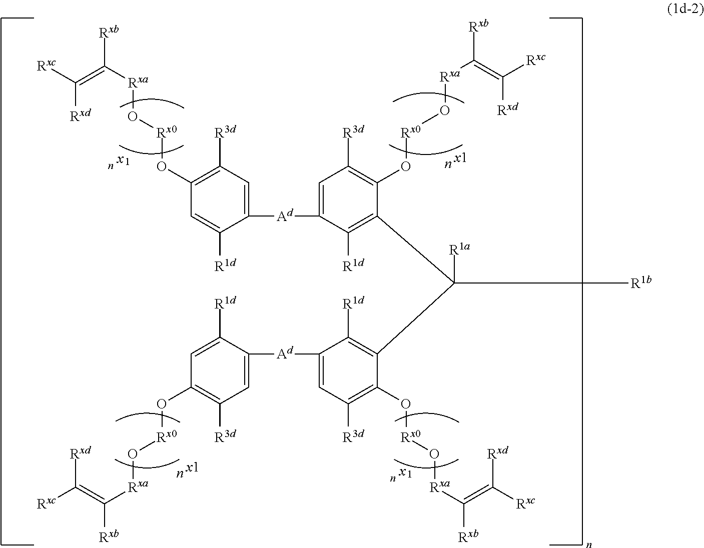

In formula (1d-2),

R^{1a}, R^{1b}, and n are each as defined in the above formula (1) or the above formula (1a); R^{3d}, R^{1d}, and A^{d} are each as defined in the above formula (1d-1); and R^{x0}, n^{x1}, R^{xa}, R^{xb}, R^{xc}, and R^{xd} are each as defined in the above formula (X).

Compound (1) of the present embodiment is preferably a compound represented by the following formula (1d-3) (hereinafter, also referred to as “compound (1d-3)”) from the viewpoint of more significantly obtaining the effect of the present invention.

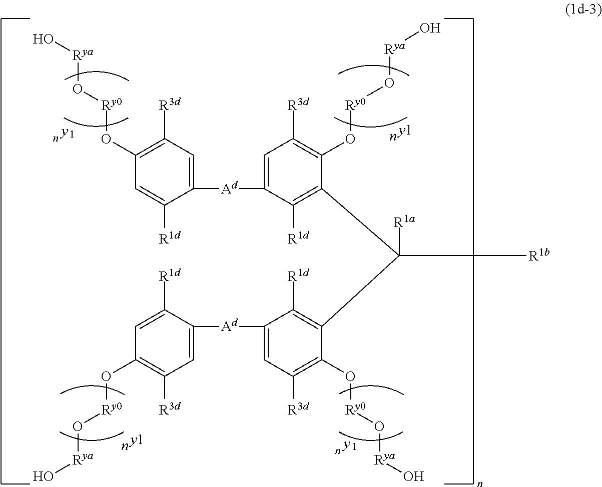

In formula (1d-3),

R^{1a}, R^{1b}, and n are each as defined in the above formula (1) or the above formula (1a); R^{3d}, R^{1d}, and A^{d} are each as defined in the above formula (1d-1); and R^{y0}, n^{y1}, and R^{ya} are each as defined in the above formula (Y1).

Compound (1) of the present embodiment is preferably a compound represented by the following formula (1d-4) (hereinafter, also referred to as “compound (1d-4)”) from the viewpoint of more significantly obtaining the effect of the present invention.

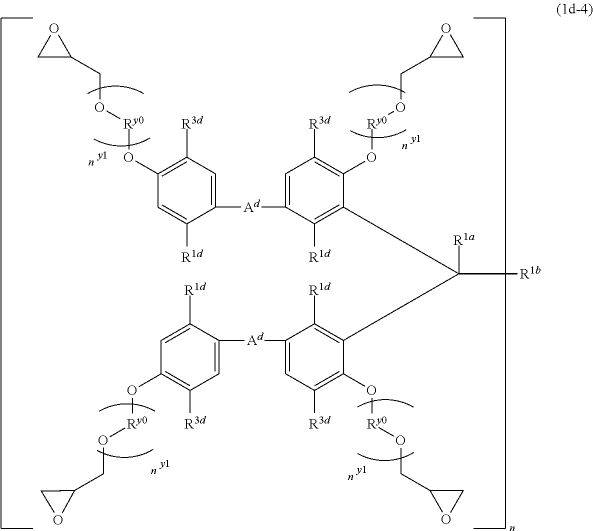

In formula (1d-4),

R^{1a}, R^{1b}, and n are each as defined in the above formula (1) or the above formula (1a); R^{3d}, R^{1d}, and A^{d} are each as defined in the above formula (1d-1); and R^{y0} and n^{y1} are each as defined in the above formula (Y1).

Compound (1) of the present embodiment is preferably a compound represented by the following formula (1d-5) (hereinafter, also referred to as “compound (1d-5)”) from the viewpoint of more significantly obtaining the effect of the present invention.

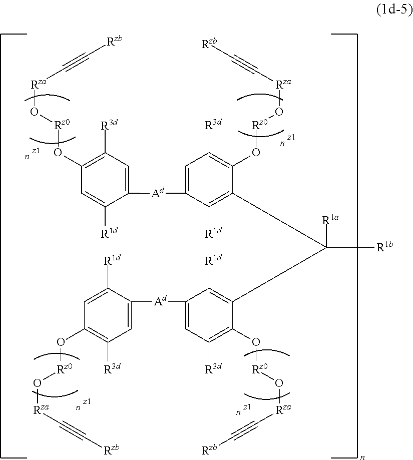

In formula (1d-5),

R^{1a}, R^{1b}, and n are each as defined in the above formula (1) or the above formula (1a); R^{3d}, R^{1d}, and A^{d} are each as defined in the above formula (1d-1); and R^{z0}, n^{z1}, R^{za}, and R^{zb} are each as defined in the above formula (Z).

Compound (1) of the present embodiment is preferably a compound represented by the following formula (1d-6) (hereinafter, also referred to as “compound (1d-6)”) from the viewpoint of more significantly obtaining the effect of the present invention.

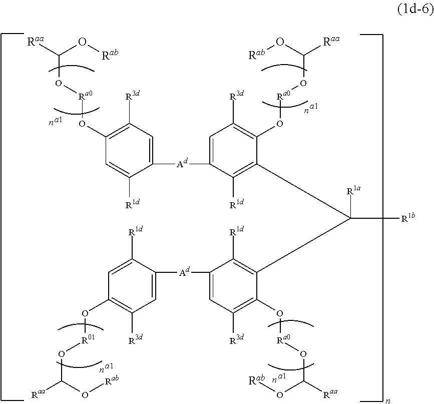

In formula (1d-6),

R^{1a}, R^{1b}, and n are each as defined in the above formula (1) or the above formula (1a); R^{1d}, R^{1d}, and A^{d} are each as defined in the above formula (1d-1); and R^{a0}, n^{a1}, R^{aa}, and R^{ab} are each as defined in the above formula (A).

Compound (1) of the present embodiment is preferably a compound represented by the following formula (1d-7) (hereinafter, also referred to as “compound (1d-7)”) from the viewpoint of more significantly obtaining the effect of the present invention.

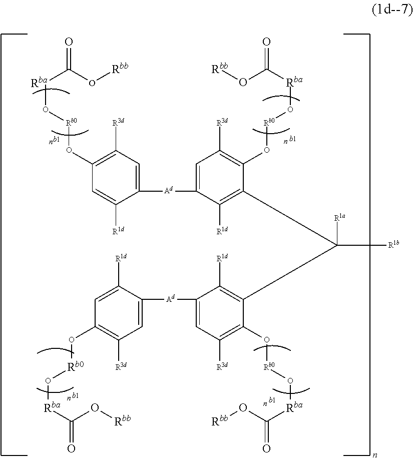

In formula (1d-7),

R^{1a}, R^{1b}, and n are each as defined in the above formula (1) or the above formula (1a); R^{3d}, R^{1d}, and A^{d} are each as defined in the above formula (1d-1); and R^{b0}, n^{b1}, R^{ba}, and R^{bb} are each as defined in the above formula (B).

Although the specific examples of compound (1) of the present embodiment are not particularly limited, examples thereof include the compounds represented by the following formulae.

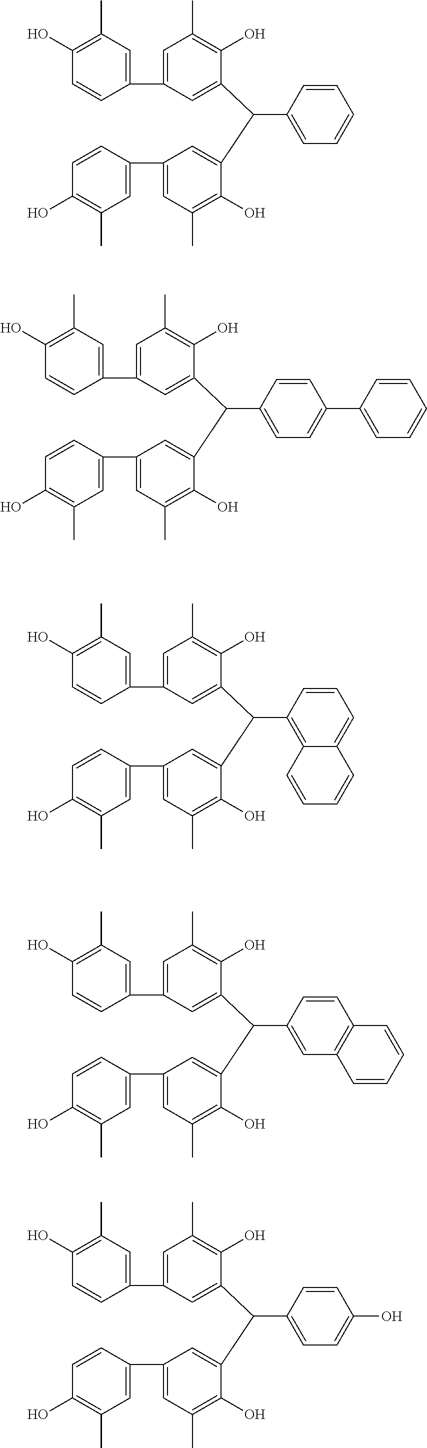

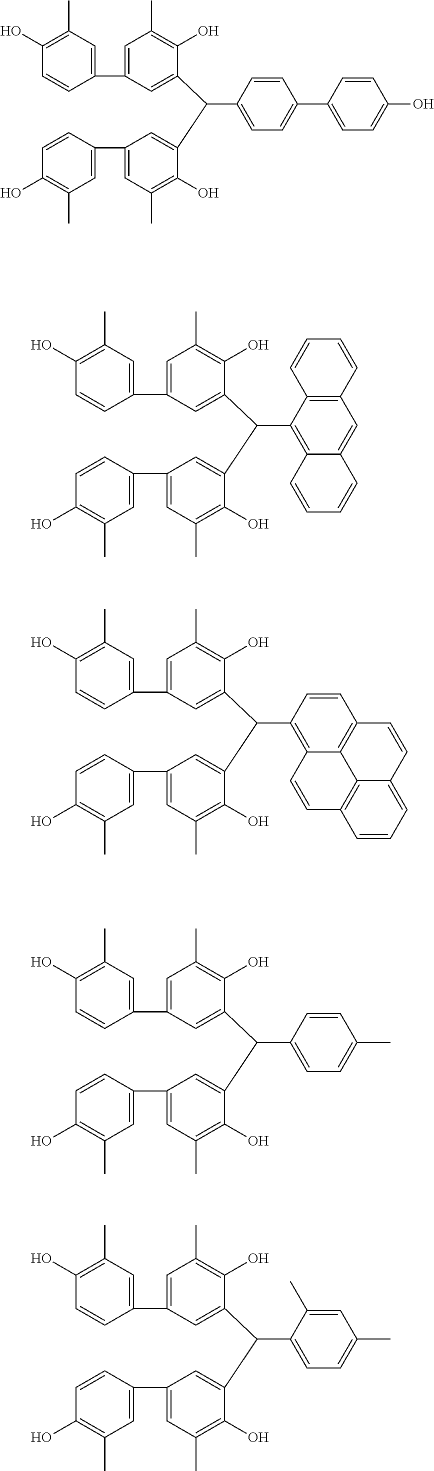 [SMILES: Cc1cc(-c2cc(C)c(O)c(C(c3ccc(-c4ccc(O)cc4)cc3)c3cc(-c4ccc(O)c(C)c4)cc(C)c3O)c2)ccc1O ; Cc1cc(-c2cc(C)c(O)c(C(c3cc(-c4ccc(O)c(C)c4)cc(C)c3O)c3c4ccccc4cc4ccccc34)c2)ccc1O ; Cc1cc(-c2cc(C)c(O)c(C(c3cc(-c4ccc(O)c(C)c4)cc(C)c3O)c3ccc4ccc5cccc6ccc3c4c56)c2)ccc1O ; Cc1ccc(C(c2cc(-c3ccc(O)c(C)c3)cc(C)c2O)c2cc(-c3ccc(O)c(C)c3)cc(C)c2O)cc1 ; Cc1ccc(C(c2cc(-c3ccc(O)c(C)c3)cc(C)c2O)c2cc(-c3ccc(O)c(C)c3)cc(C)c2O)c(C)c1]

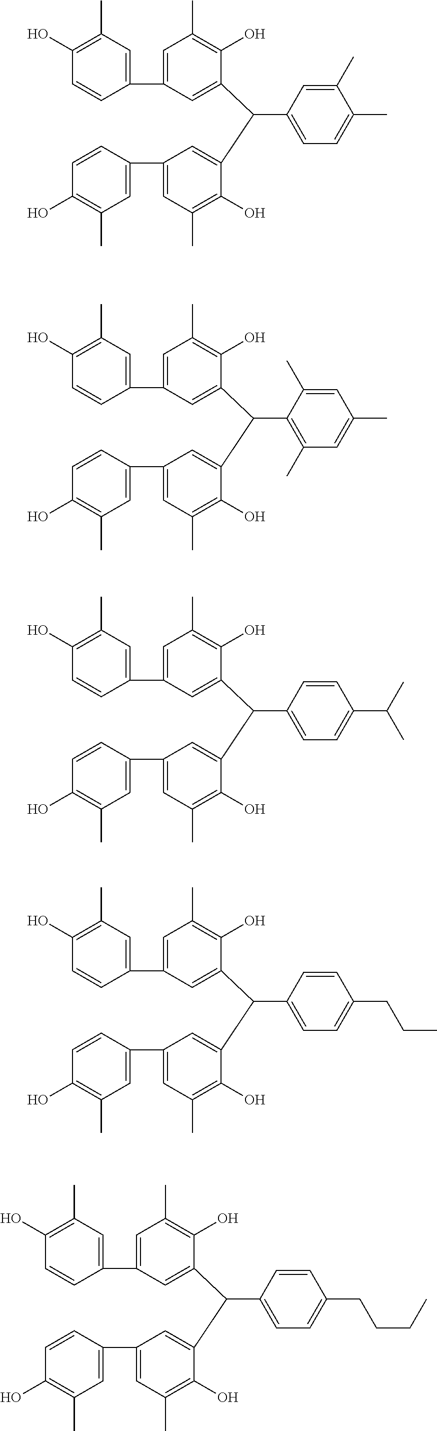

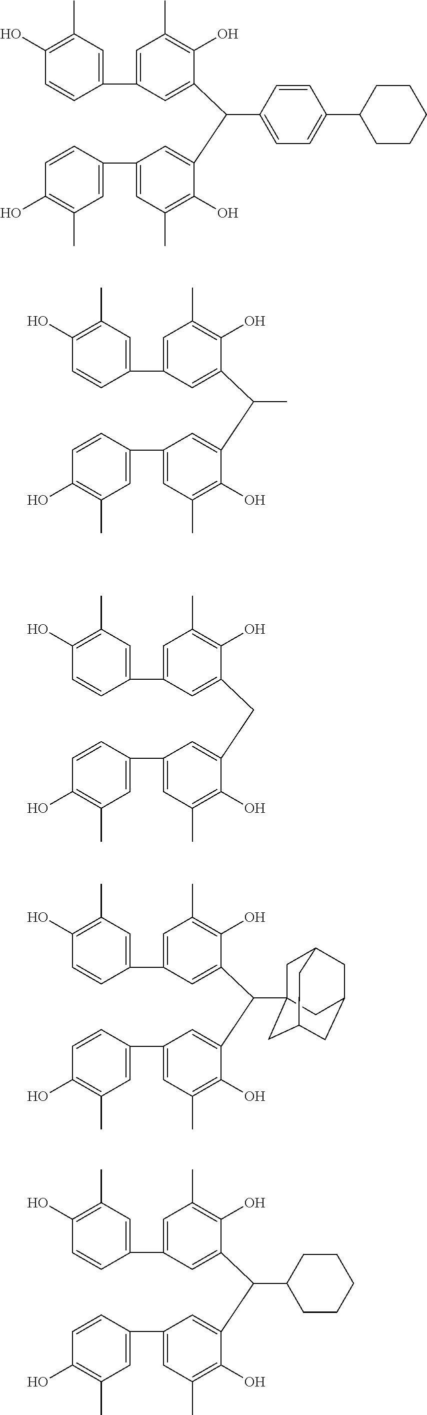

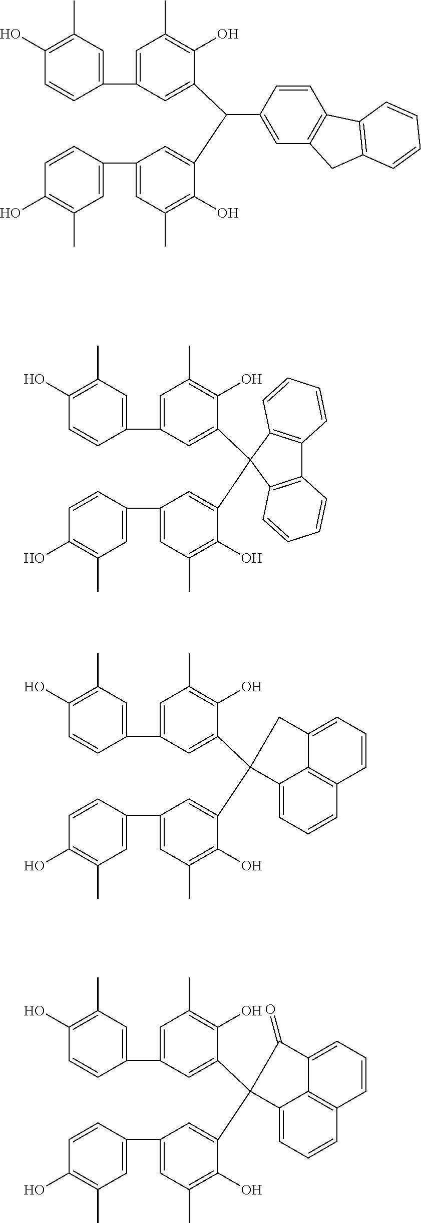 [SMILES: Cc1cc(-c2cc(C)c(O)c(C(c3ccc4c(c3)Cc3ccccc3-4)c3cc(-c4ccc(O)c(C)c4)cc(C)c3O)c2)ccc1O ; Cc1cc(-c2cc(C)c(O)c(C3(c4cc(-c5ccc(O)c(C)c5)cc(C)c4O)c4ccccc4-c4ccccc43)c2)ccc1O ; Cc1cc(-c2cc(C)c(O)c(C3(c4cc(-c5ccc(O)c(C)c5)cc(C)c4O)Cc4cccc5cccc3c45)c2)ccc1O ; Cc1cc(-c2cc(C)c(O)c(C3(c4cc(-c5ccc(O)c(C)c5)cc(C)c4O)C(=O)c4cccc5cccc3c45)c2)ccc1O]

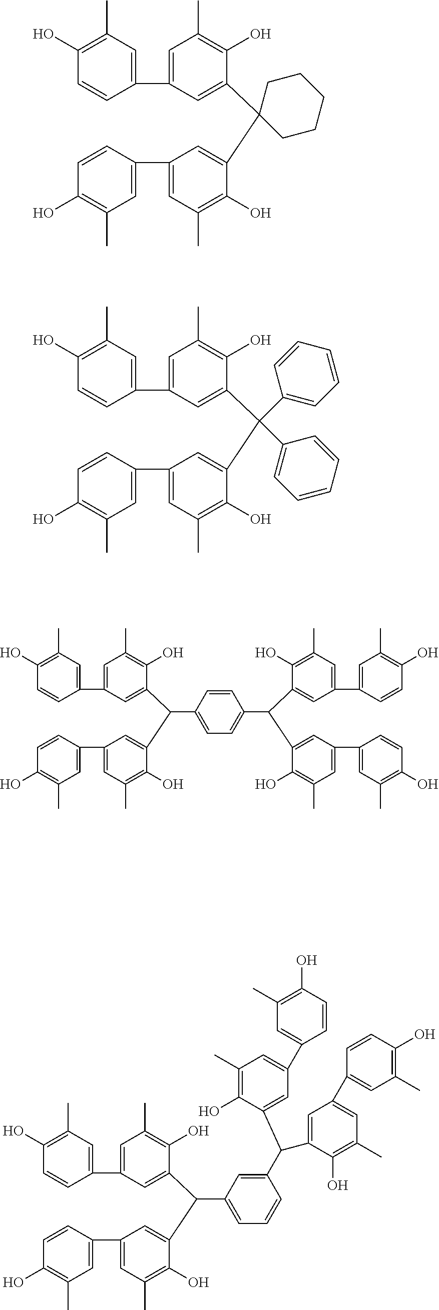

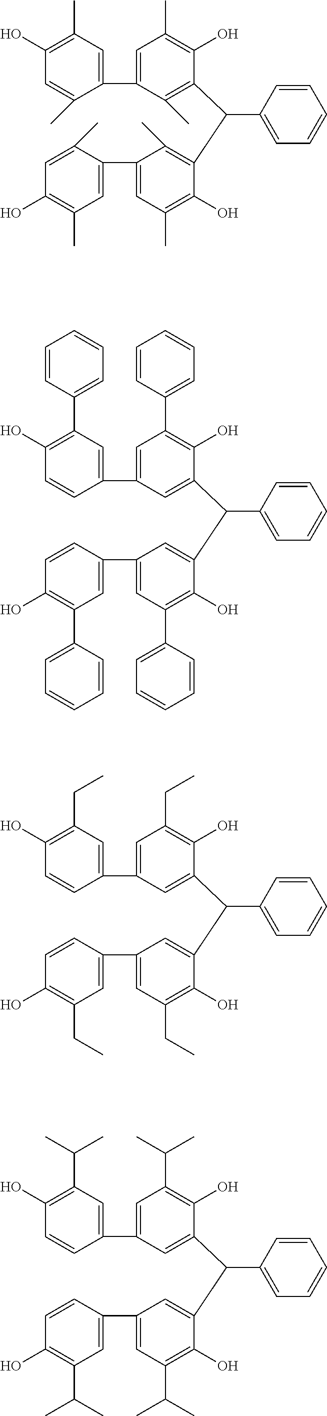 [SMILES: Cc1cc(-c2cc(C)c(O)c(C(c3ccccc3)c3c(C)c(-c4cc(C)c(O)cc4C)cc(C)c3O)c2C)c(C)cc1O ; Oc1ccc(-c2cc(-c3ccccc3)c(O)c(C(c3ccccc3)c3cc(-c4ccc(O)c(-c5ccccc5)c4)cc(-c4ccccc4)c3O)c2)cc1-c1ccccc1 ; CCc1cc(-c2cc(CC)c(O)c(C(c3ccccc3)c3cc(-c4ccc(O)c(CC)c4)cc(CC)c3O)c2)ccc1O ; CC(C)c1cc(-c2cc(C(C)C)c(O)c(C(c3ccccc3)c3cc(-c4ccc(O)c(C(C)C)c4)cc(C(C)C)c3O)c2)ccc1O]

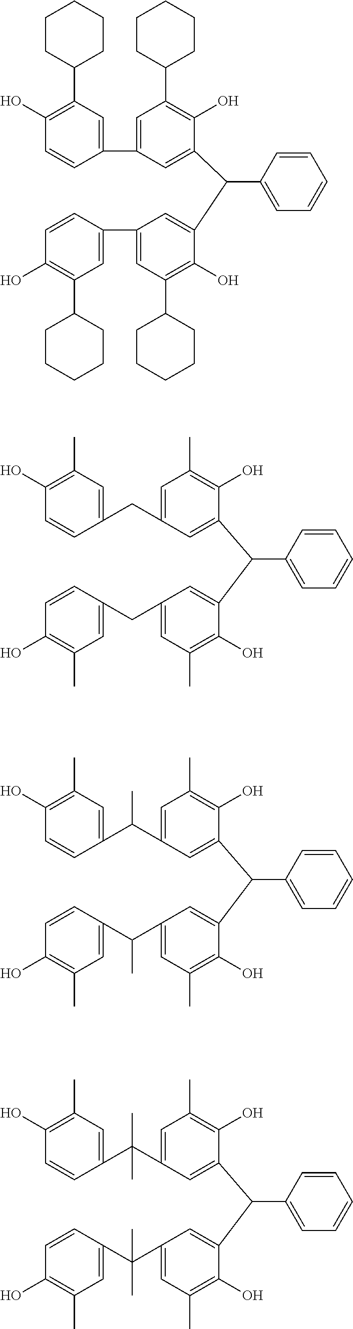 [SMILES: Oc1ccc(-c2cc(C3CCCCC3)c(O)c(C(c3ccccc3)c3cc(-c4ccc(O)c(C5CCCCC5)c4)cc(C4CCCCC4)c3O)c2)cc1C1CCCCC1 ; Cc1cc(Cc2cc(C)c(O)c(C(c3ccccc3)c3cc(Cc4ccc(O)c(C)c4)cc(C)c3O)c2)ccc1O ; Cc1cc(C(C)c2cc(C)c(O)c(C(c3ccccc3)c3cc(C(C)c4ccc(O)c(C)c4)cc(C)c3O)c2)ccc1O ; Cc1cc(C(C)(C)c2cc(C)c(O)c(C(c3ccccc3)c3cc(C(C)(C)c4ccc(O)c(C)c4)cc(C)c3O)c2)ccc1O]

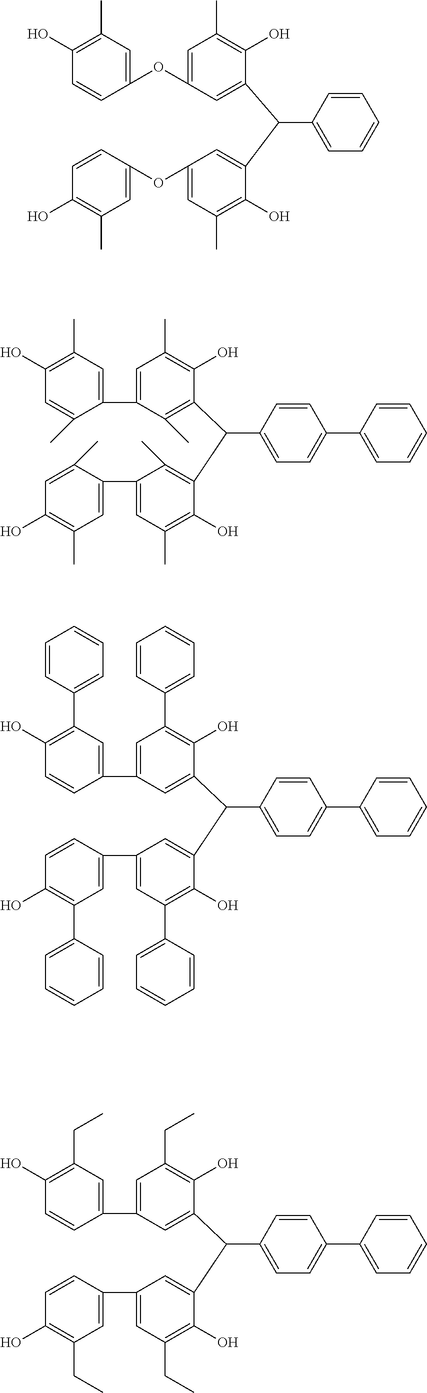 [SMILES: Cc1cc(Oc2cc(C)c(O)c(C(c3ccccc3)c3cc(Oc4ccc(O)c(C)c4)cc(C)c3O)c2)ccc1O ; Cc1cc(-c2cc(C)c(O)c(C(c3ccc(-c4ccccc4)cc3)c3c(C)c(-c4cc(C)c(O)cc4C)cc(C)c3O)c2C)c(C)cc1O ; Oc1ccc(-c2cc(-c3ccccc3)c(O)c(C(c3ccc(-c4ccccc4)cc3)c3cc(-c4ccc(O)c(-c5ccccc5)c4)cc(-c4ccccc4)c3O)c2)cc1-c1ccccc1 ; CCc1cc(-c2cc(CC)c(O)c(C(c3ccc(-c4ccccc4)cc3)c3cc(-c4ccc(O)c(CC)c4)cc(CC)c3O)c2)ccc1O]

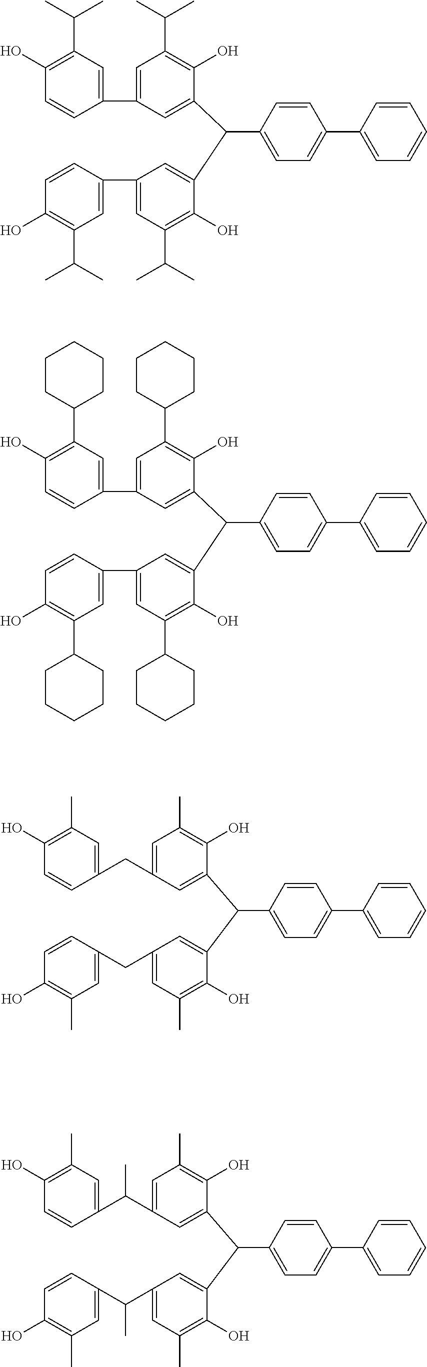 [SMILES: CC(C)c1cc(-c2cc(C(C)C)c(O)c(C(c3ccc(-c4ccccc4)cc3)c3cc(-c4ccc(O)c(C(C)C)c4)cc(C(C)C)c3O)c2)ccc1O ; Oc1ccc(-c2cc(C3CCCCC3)c(O)c(C(c3ccc(-c4ccccc4)cc3)c3cc(-c4ccc(O)c(C5CCCCC5)c4)cc(C4CCCCC4)c3O)c2)cc1C1CCCCC1 ; Cc1cc(Cc2cc(C)c(O)c(C(c3ccc(-c4ccccc4)cc3)c3cc(Cc4ccc(O)c(C)c4)cc(C)c3O)c2)ccc1O ; Cc1cc(C(C)c2cc(C)c(O)c(C(c3ccc(-c4ccccc4)cc3)c3cc(C(C)c4ccc(O)c(C)c4)cc(C)c3O)c2)ccc1O]

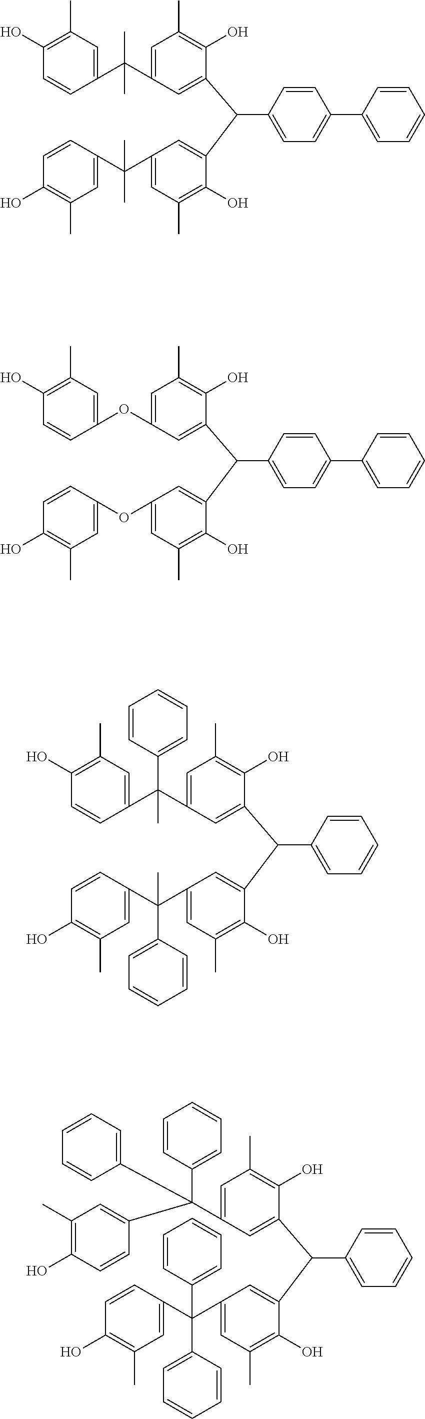 [SMILES: Cc1cc(C(C)(C)c2cc(C)c(O)c(C(c3ccc(-c4ccccc4)cc3)c3cc(C(C)(C)c4ccc(O)c(C)c4)cc(C)c3O)c2)ccc1O ; Cc1cc(Oc2cc(C)c(O)c(C(c3ccc(-c4ccccc4)cc3)c3cc(Oc4ccc(O)c(C)c4)cc(C)c3O)c2)ccc1O ; Cc1cc(C(C)(c2ccccc2)c2cc(C)c(O)c(C(c3ccccc3)c3cc(C(C)(c4ccccc4)c4ccc(O)c(C)c4)cc(C)c3O)c2)ccc1O ; Cc1cc(C(c2ccccc2)(c2ccccc2)c2cc(C)c(O)c(C(c3ccccc3)c3cc(C(c4ccccc4)(c4ccccc4)c4ccc(O)c(C)c4)cc(C)c3O)c2)ccc1O]

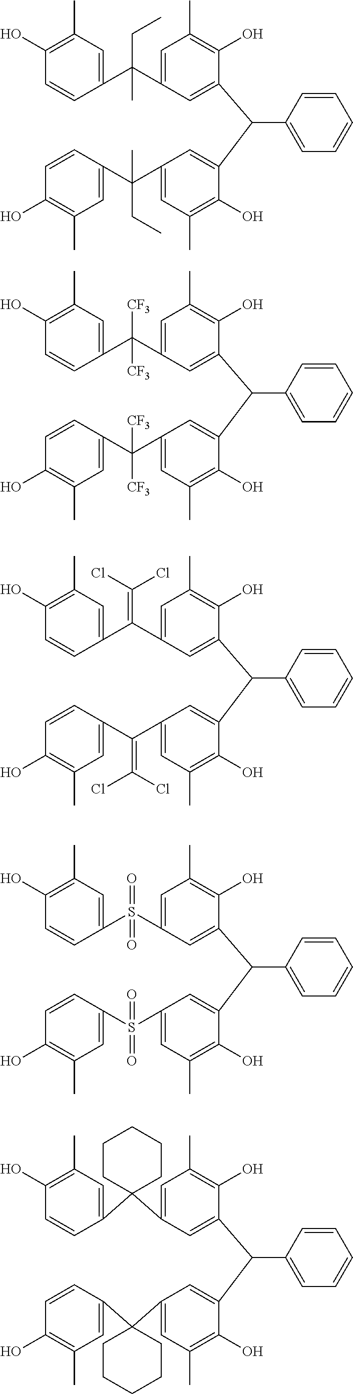

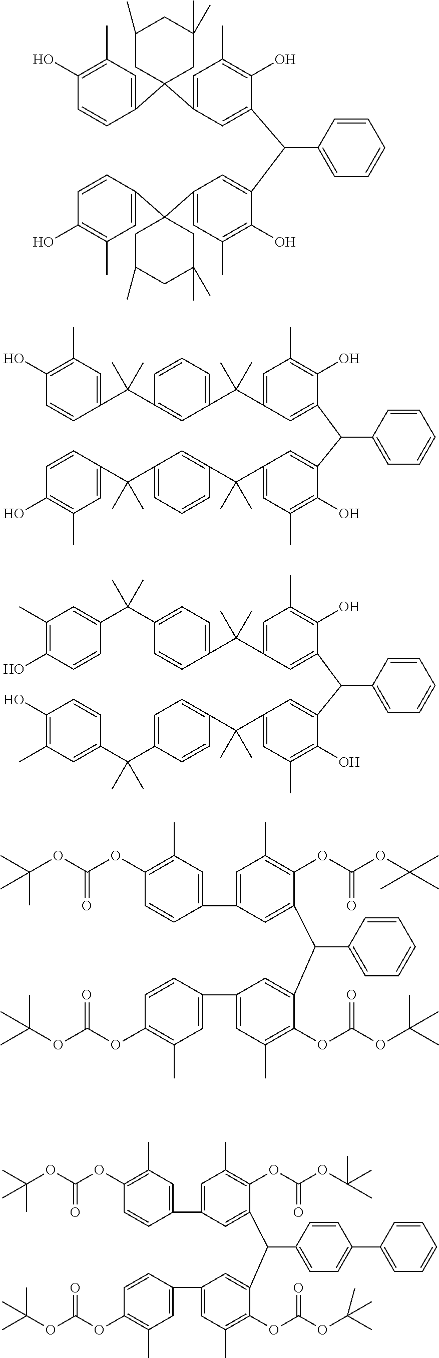

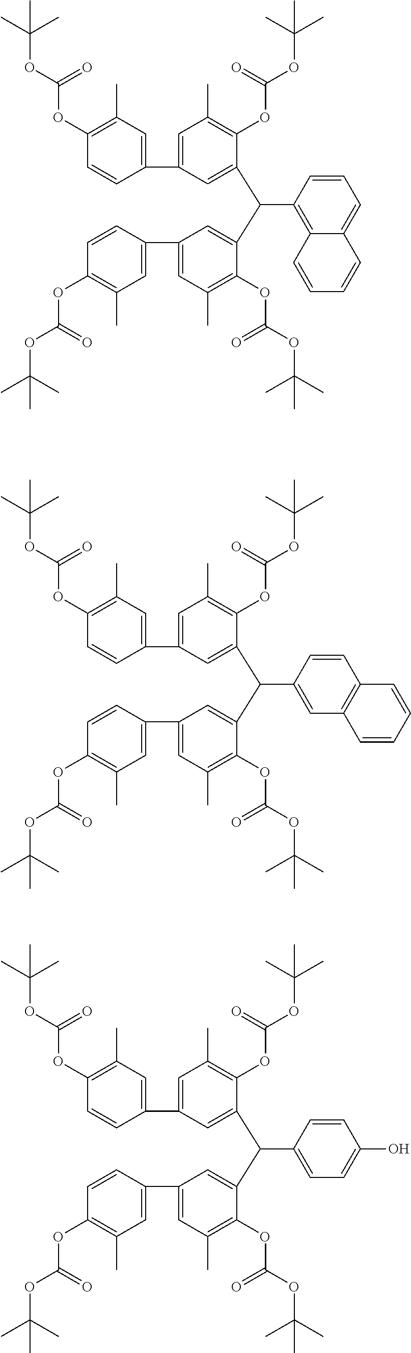 [SMILES: Cc1cc(-c2cc(C)c(OC(=O)OC(C)(C)C)c(C(c3cc(-c4ccc(OC(=O)OC(C)(C)C)c(C)c4)cc(C)c3OC(=O)OC(C)(C)C)c3cccc4ccccc34)c2)ccc1OC(=O)OC(C)(C)C ; Cc1cc(-c2cc(C)c(OC(=O)OC(C)(C)C)c(C(c3ccc4ccccc4c3)c3cc(-c4ccc(OC(=O)OC(C)(C)C)c(C)c4)cc(C)c3OC(=O)OC(C)(C)C)c2)ccc1OC(=O)OC(C)(C)C ; Cc1cc(-c2cc(C)c(OC(=O)OC(C)(C)C)c(C(c3ccc(O)cc3)c3cc(-c4ccc(OC(=O)OC(C)(C)C)c(C)c4)cc(C)c3OC(=O)OC(C)(C)C)c2)ccc1OC(=O)OC(C)(C)C]

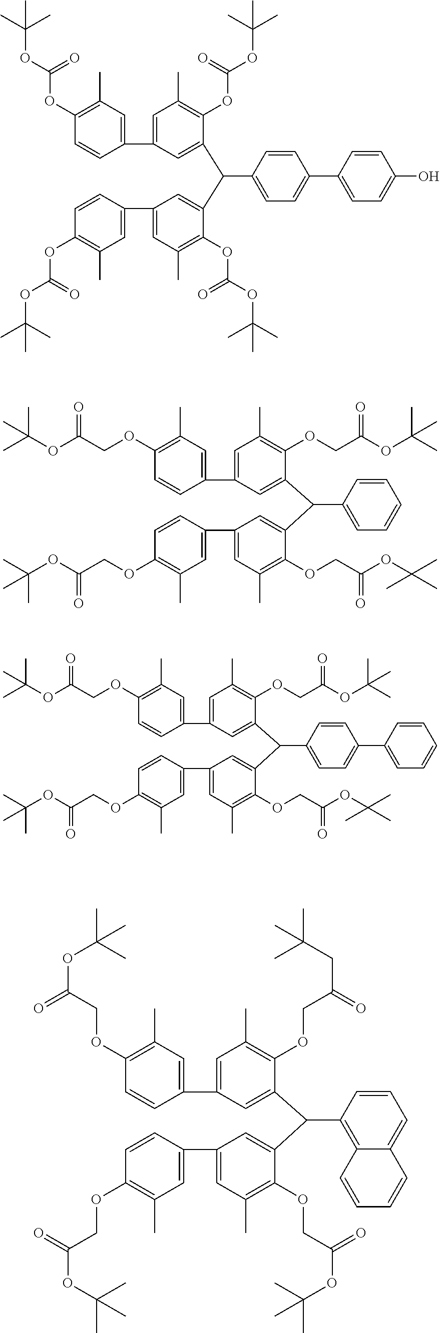

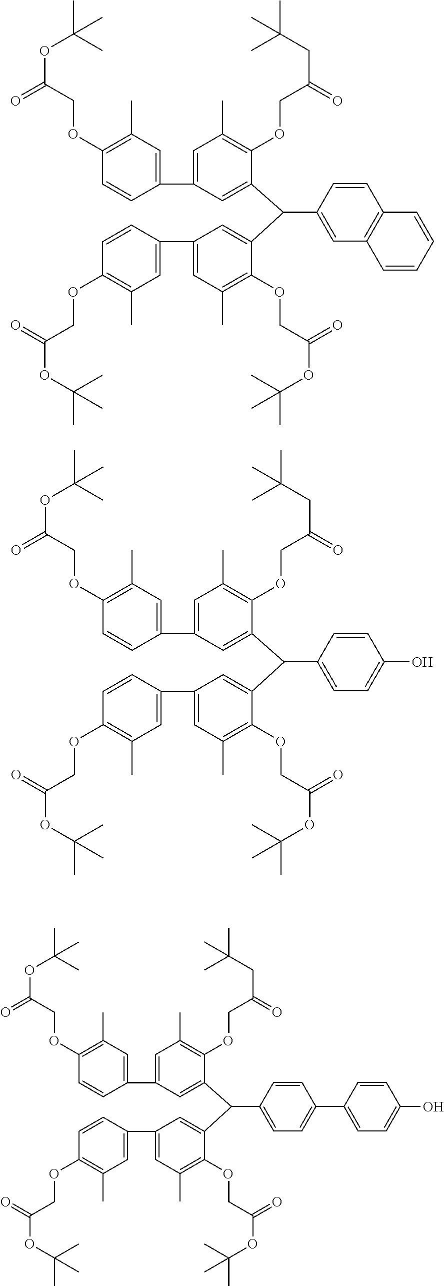

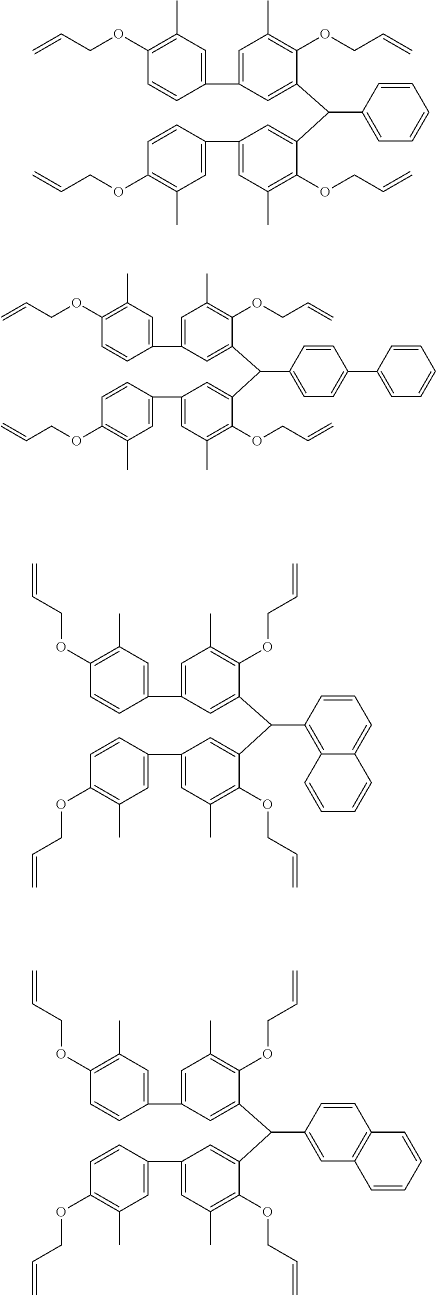 [SMILES: C=CCOc1ccc(-c2cc(C)c(OCC=C)c(C(c3ccccc3)c3cc(-c4ccc(OCC=C)c(C)c4)cc(C)c3OCC=C)c2)cc1C ; C=CCOc1ccc(-c2cc(C)c(OCC=C)c(C(c3ccc(-c4ccccc4)cc3)c3cc(-c4ccc(OCC=C)c(C)c4)cc(C)c3OCC=C)c2)cc1C ; C=CCOc1ccc(-c2cc(C)c(OCC=C)c(C(c3cc(-c4ccc(OCC=C)c(C)c4)cc(C)c3OCC=C)c3cccc4ccccc34)c2)cc1C ; C=CCOc1ccc(-c2cc(C)c(OCC=C)c(C(c3ccc4ccccc4c3)c3cc(-c4ccc(OCC=C)c(C)c4)cc(C)c3OCC=C)c2)cc1C]

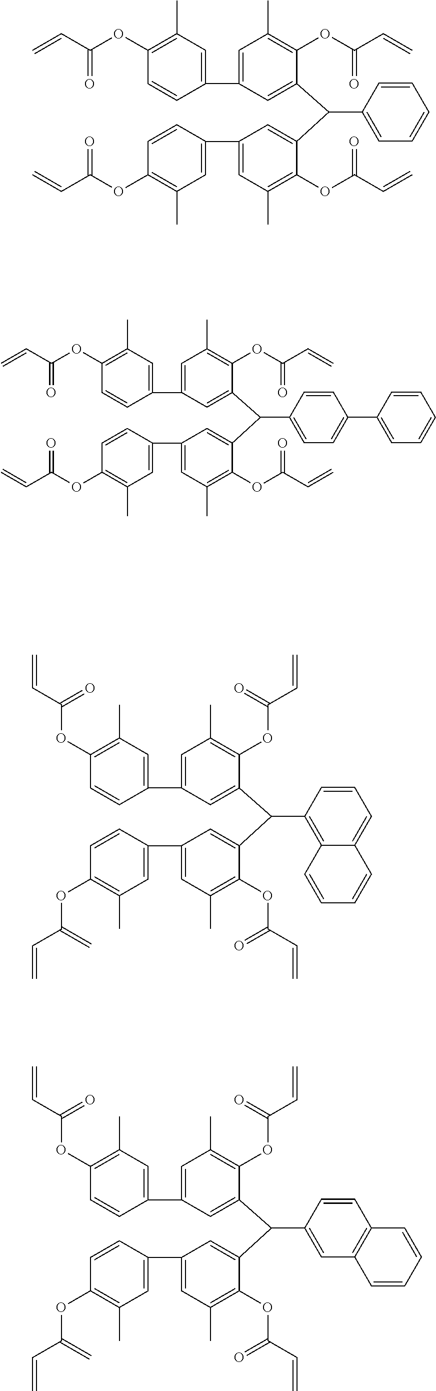 [SMILES: C=CC(=O)Oc1ccc(-c2cc(C)c(OC(=O)C=C)c(C(c3ccccc3)c3cc(-c4ccc(OC(=O)C=C)c(C)c4)cc(C)c3OC(=O)C=C)c2)cc1C ; C=CC(=O)Oc1ccc(-c2cc(C)c(OC(=O)C=C)c(C(c3ccc(-c4ccccc4)cc3)c3cc(-c4ccc(OC(=O)C=C)c(C)c4)cc(C)c3OC(=O)C=C)c2)cc1C ; C=CC(=C)Oc1ccc(-c2cc(C)c(OC(=O)C=C)c(C(c3cc(-c4ccc(OC(=O)C=C)c(C)c4)cc(C)c3OC(=O)C=C)c3cccc4ccccc34)c2)cc1C ; C=CC(=C)Oc1ccc(-c2cc(C)c(OC(=O)C=C)c(C(c3ccc4ccccc4c3)c3cc(-c4ccc(OC(=O)C=C)c(C)c4)cc(C)c3OC(=O)C=C)c2)cc1C]

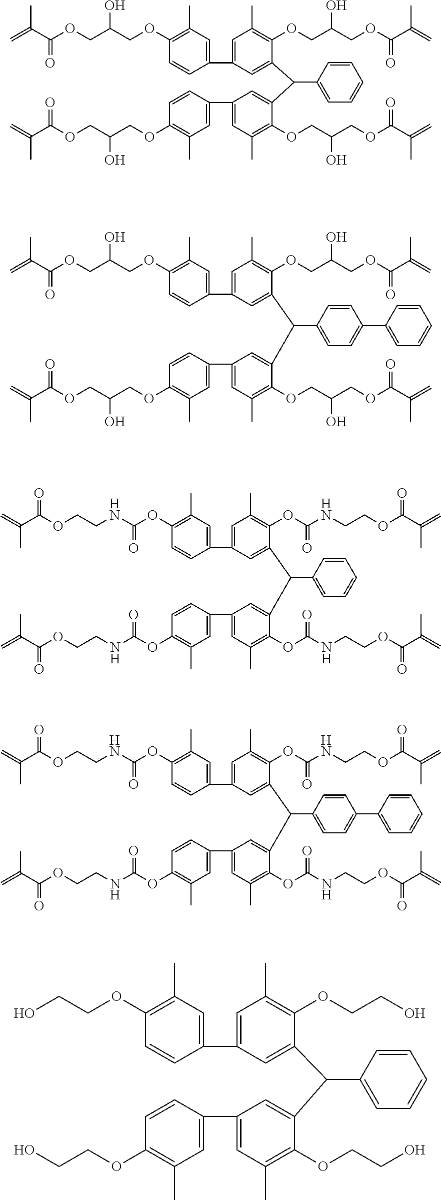

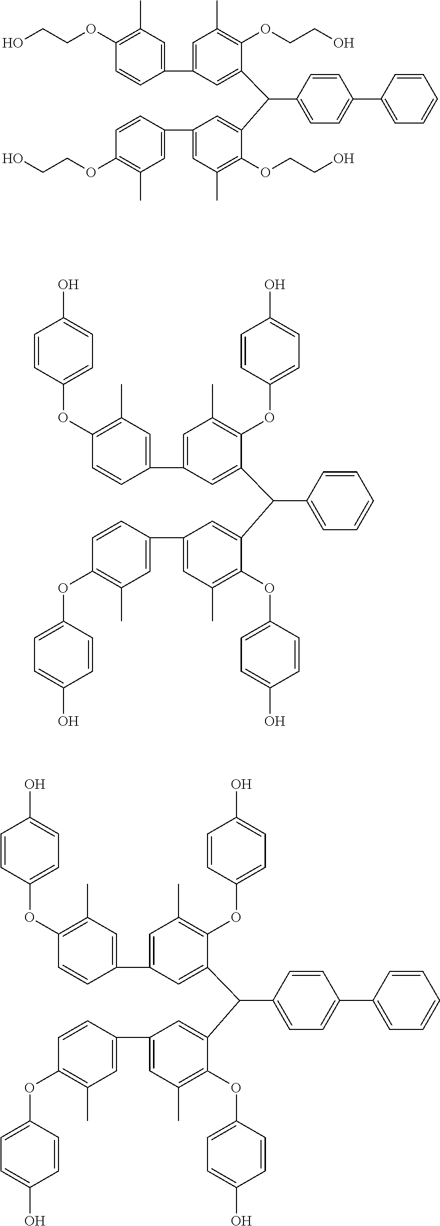 [SMILES: Cc1cc(-c2cc(C)c(OCCO)c(C(c3ccc(-c4ccccc4)cc3)c3cc(-c4ccc(OCCO)c(C)c4)cc(C)c3OCCO)c2)ccc1OCCO ; Cc1cc(-c2cc(C)c(Oc3ccc(O)cc3)c(C(c3ccccc3)c3cc(-c4ccc(Oc5ccc(O)cc5)c(C)c4)cc(C)c3Oc3ccc(O)cc3)c2)ccc1Oc1ccc(O)cc1 ; Cc1cc(-c2cc(C)c(Oc3ccc(O)cc3)c(C(c3ccc(-c4ccccc4)cc3)c3cc(-c4ccc(Oc5ccc(O)cc5)c(C)c4)cc(C)c3Oc3ccc(O)cc3)c2)ccc1Oc1ccc(O)cc1]

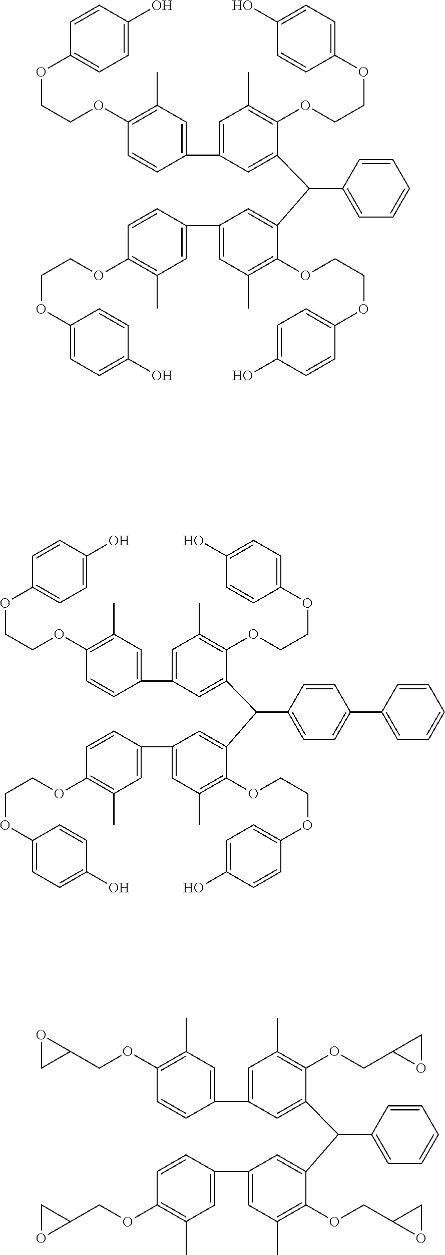

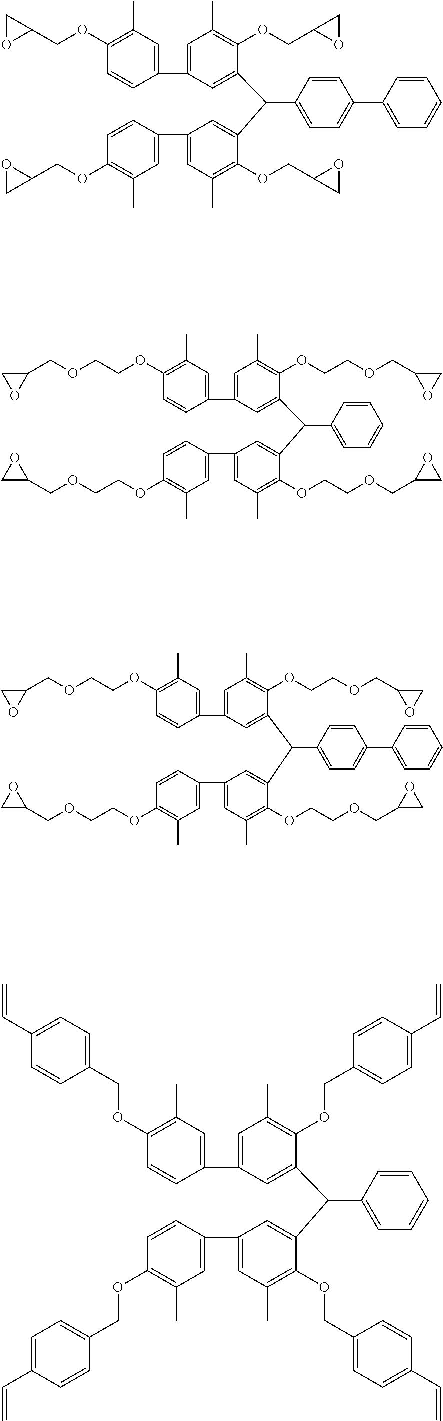

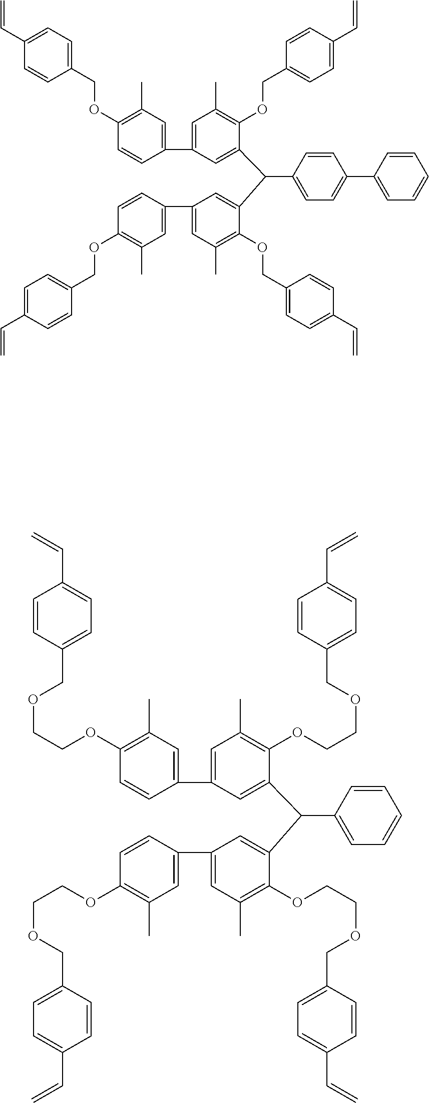 [SMILES: C=Cc1ccc(COc2ccc(-c3cc(C)c(OCc4ccc(C=C)cc4)c(C(c4ccc(-c5ccccc5)cc4)c4cc(-c5ccc(OCc6ccc(C=C)cc6)c(C)c5)cc(C)c4OCc4ccc(C=C)cc4)c3)cc2C)cc1 ; C=Cc1ccc(COCCOc2ccc(-c3cc(C)c(OCCOCc4ccc(C=C)cc4)c(C(c4ccccc4)c4cc(-c5ccc(OCCOCc6ccc(C=C)cc6)c(C)c5)cc(C)c4OCCOCc4ccc(C=C)cc4)c3)cc2C)cc1]

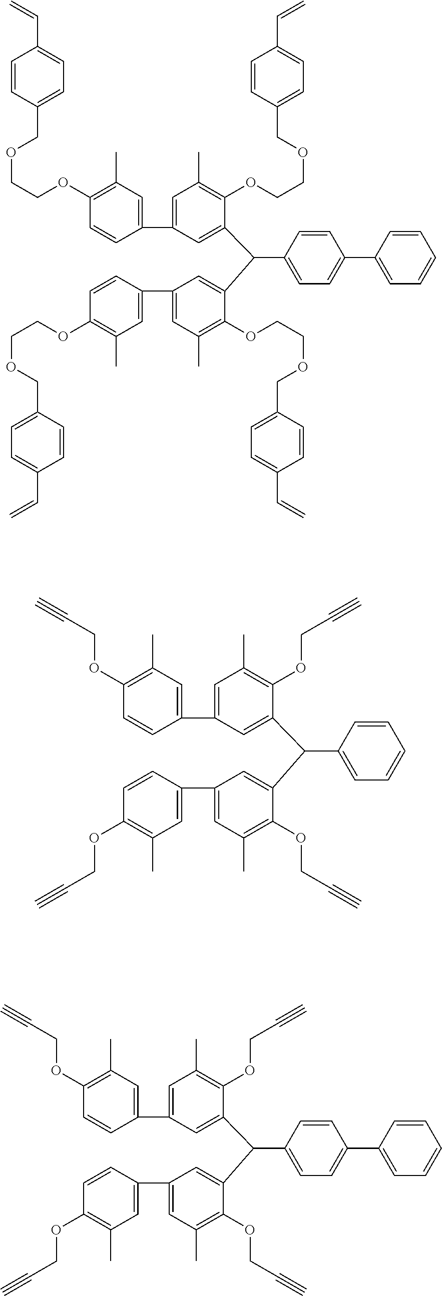 [SMILES: C=Cc1ccc(COCCOc2ccc(-c3cc(C)c(OCCOCc4ccc(C=C)cc4)c(C(c4ccc(-c5ccccc5)cc4)c4cc(-c5ccc(OCCOCc6ccc(C=C)cc6)c(C)c5)cc(C)c4OCCOCc4ccc(C=C)cc4)c3)cc2C)cc1 ; C#CCOc1ccc(-c2cc(C)c(OCC#C)c(C(c3ccccc3)c3cc(-c4ccc(OCC#C)c(C)c4)cc(C)c3OCC#C)c2)cc1C ; C#CCOc1ccc(-c2cc(C)c(OCC#C)c(C(c3ccc(-c4ccccc4)cc3)c3cc(-c4ccc(OCC#C)c(C)c4)cc(C)c3OCC#C)c2)cc1C]

### [Method for Producing Compound (1)]

Examples of a method for synthesizing compound (1) of the present embodiment include, but not particularly limited to, the following method. That is, compound (1) is obtained through a polycondensation reaction, at normal pressure, among a compound represented by the following formula (1-x) (hereinafter, compound (1-x)), a compound represented by the following formula (1-y) (hereinafter, compound (1-y)), and a compound represented by the following formula (z1) (hereinafter, compound (z1)), compound represented by the following formula (z2) (hereinafter, compound (z2)) or a precursor thereof in the presence of an acid catalyst or base catalyst. If necessary, the above reaction may be carried out under increased pressure. Compound (1-x) is preferably a compound represented by the following formula (1-x1), and compound (1-y) is a compound represented by the following formula (1-y1).

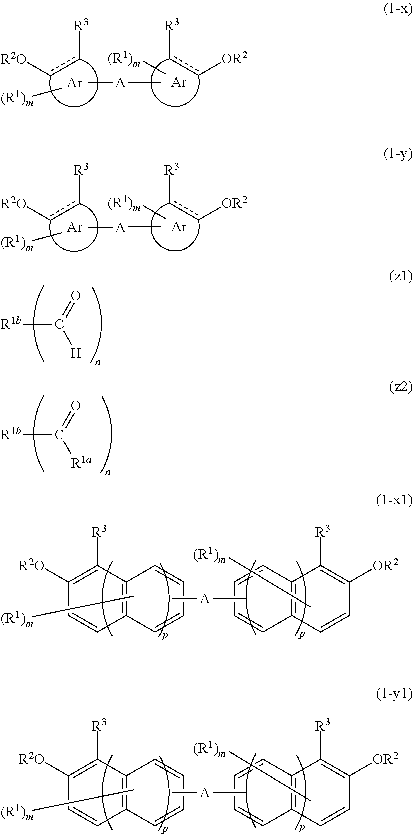

In the above formula (1-x) and formula (1-x1), A, R¹, R², R³, m, and p are each as defined in formula (1) or formula (1-1). In the above formula (1-y) and formula (1-y1), A, R¹, R², R³, m, and p are each as defined in formula (1) or formula (1-1). The above compound (1-x) and the above compound (1-y) may be identical.

In the above formula (z1), R^{1b} and n are each as defined in the above formula (1) or the above formula (1a). In the above formula (z2), R^{1a}, R^{1b}, and n are each as defined in the above formula (1) or the above formula (1a).

Although specific examples of the above polycondensation reaction are not particularly limited, compound (1) is obtained through a polycondensation reaction of compound (1-x) and compound (1-y) with compound (z1), compound (z2), or precursors thereof in the presence of an acid catalyst or base catalyst.

Examples of compound (1-x) and compound (1-y) include, but not particularly limited to, 3,3′-dimethylbiphenyl-4,4′-diol, 2,2′,5,5′-tetramethylbiphenyl-4,4′-diol, 3,3′-diphenylbiphenyl-4,4′-diol, 2,2′,5,5′-tetraphenylbiphenyl-4,4′-diol, and methylenebis(3-methyl-4-phenol). These compounds are used alone as one kind or in combination of two or more kinds. Among these compounds, 3,3′-dimethylbiphenyl-4,4′-diol and 3,3′-diphenylbiphenyl-4,4′-diol are preferable.

Examples of compound (z1) and its precursor include, but not particularly limited to, formaldehyde, trioxane, paraformaldehyde, benzaldehyde, acetaldehyde, propylaldehyde, phenylacetaldehyde, phenylpropionaldehyde, hydroxybenzaldehyde, chlorobenzaldehyde, nitrobenzaldehyde, methylbenzaldehyde, dimethylbenzaldehyde, trimethylbenzaldehyde, pentamethylbenzaldehyde, ethylbenzaldehyde, propylbenzaldehyde, butylbenzaldehyde, pentylbenzaldehyde, butylmethylbenzaldehyde, hydroxybenzaldehyde, dihydroxybenzaldehyde, fluoromethylbenzaldehyde, cyclopropanecarbaldehyde, cyclobutanecarbaldehyde, cyclohexanecarbaldehyde, cyclodecanecarbaldehyde, cycloundecanecarbaldehyde, cyclopropylbenzaldehyde, cyclobutylbenzaldehyde, cyclohexylbenzaldehyde, cyclodecylbenzaldehyde, cycloundecylbenzaldehyde, biphenylaldehyde, naphthaldehyde, anthracenecarbaldehyde, phenanthrenecarbaldehyde, pyrenecarbaldehyde, and furfural. These aldehydes are used alone as one kind or in combination of two or more kinds. Among them, it is preferable to use one or more selected from the group consisting of benzaldehyde, phenylacetaldehyde, phenylpropylaldehyde, hydroxybenzaldehyde, chlorobenzaldehyde, nitrobenzaldehyde, methylbenzaldehyde, ethylbenzaldehyde, butylbenzaldehyde, cyclohexylbenzaldehyde, biphenylaldehyde, naphthaldehyde, anthracenecarbaldehyde, phenanthrenecarbaldehyde, pyrenecarbaldehyde and furfural from the viewpoint that high heat resistance can be exhibited, and it is more preferable to use one or more selected from the group consisting of benzaldehyde, hydroxybenzaldehyde, chlorobenzaldehyde, nitrobenzaldehyde, methylbenzaldehyde, ethylbenzaldehyde, butylbenzaldehyde, cyclohexylbenzaldehyde, biphenylaldehyde, naphthaldehyde, anthracenecarbaldehyde, phenanthrenecarbaldehyde, pyrenecarbaldehyde and furfural from the viewpoint of improving etching resistance.

Examples of compound (z2) include, but not particularly limited to, acetone, methyl ethyl ketone, cyclobutanone, cyclopentanone, cyclohexanone, norbornanone, cyclohexanedione, cyclohexanetrione, cyclodecanetrione, adamantanone, fluorenone, benzofluorenone, dibenzofluorenone, acenaphthenequinone, acenaphthenone, anthraquinone, acetophenone, diacetylbenzene, triacetylbenzene, acetonaphthone, acetylmethylbenzene, acetyldimethylbenzene, acetyltrimethylbenzene, acetylethylbenzene, acetylpropylbenzene, acetylbutylbenzene, acetylpentabenzene, acetylbutylmethylbenzene, acetylhydroxybenzene, acetyldihydroxybenzene, acetylfluoromethylbenzene, diphenylcarbonylnaphthalene, phenylcarbonylbiphenyl, diphenylcarbonylbiphenyl, benzophenone, diphenylcarbonylbenzene, triphenylcarbonylbenzene, benzonaphthone, diphenylcarbonylnaphthalene, phenylcarbonylbiphenyl, and diphenylcarbonylbiphenyl. These ketones are used alone as one kind or in combination of two or more kinds. Among them, it is preferable to use one or more selected from the group consisting of cyclopentanone, cyclohexanone, norbornanone, cyclohexanedione, cyclohexanetrione, cyclodecanetrione, adamantanone, fluorenone, benzofluorenone, acenaphthenequinone, acenaphthenone, anthraquinone, acetophenone, diacetylbenzene, triacetylbenzene, acetonaphthone, diphenylcarbonylnaphthalene, phenylcarbonylbiphenyl, diphenylcarbonylbiphenyl, benzophenone, diphenylcarbonylbenzene, triphenylcarbonylbenzene, benzonaphthone, diphenylcarbonylnaphthalene, phenylcarbonylbiphenyl, and diphenylcarbonylbiphenyl from the viewpoint of enabling exhibition of high heat resistance, and it is more preferable to use one or more selected from the group consisting of acetophenone, diacetylbenzene, triacetylbenzene, acetonaphthone, diphenylcarbonylnaphthalene, phenylcarbonylbiphenyl, diphenylcarbonylbiphenyl, benzophenone, diphenylcarbonylbenzene, triphenylcarbonylbenzene, benzonaphthone, diphenylcarbonylnaphthalene, phenylcarbonylbiphenyl, and diphenylcarbonylbiphenyl from the viewpoint of improving etching resistance.

As compound (z1) or compound (z2), an aldehyde having an aromatic ring or a ketone having an aromatic ring is preferably used from the viewpoint of achieving both high heat resistance and high etching resistance.

Examples of the acid catalyst to be used in the above reaction include, but not particularly limited to, an inorganic acid such as hydrochloric acid, sulfuric acid, phosphoric acid, hydrobromic acid, and hydrofluoric acid; an organic acid such as oxalic acid, malonic acid, succinic acid, adipic acid, sebacic acid, citric acid, fumaric acid, maleic acid, formic acid, p-toluenesulfonic acid, methanesulfonic acid, trifluoroacetic acid, dichloroacetic acid, trichloroacetic acid, trifluoromethanesulfonic acid, benzenesulfonic acid, naphthalenesulfonic acid, and naphthalenedisulfonic acid; a Lewis acid such as zinc chloride, aluminum chloride, iron chloride, and boron trifluoride; and a solid acid such as tungstosilicic acid, tungstophosphoric acid, silicomolybdic acid, and phosphomolybdic acid. These acid catalysts are used alone as one kind or in combination of two or more kinds. Among them, organic acids and solid acids are preferable from the viewpoint of production, and it is preferable to use hydrochloric acid or sulfuric acid from the viewpoint of production such as easy availability and handleability. The amount of the acid catalyst used can be arbitrarily set according to, for example, the kind of the raw materials used and the catalyst used and moreover the reaction conditions and is not particularly limited, but is preferably 0.01 to 100 parts by mass based on 100 parts by mass of the reaction raw materials.

Examples of the base catalyst to be used in the above reaction include, but not particularly limited to, a metal alkoxide (e.g. an alkali metal or alkaline earth metal alkoxide such as sodium methoxide, sodium ethoxide, potassium methoxide, and potassium ethoxide), a metal hydroxide (e.g. an alkali metal or alkaline earth metal hydroxide such as sodium hydroxide and potassium hydroxide), an alkali metal or alkaline earth metal hydrogen carbonate such as sodium hydrogen carbonate and potassium hydrogen carbonate, an amine (for example, a tertiary amine (a trialkylamine such as triethylamine, an aromatic tertiary amine such as N,N-dimethylaniline, and a heterocyclic tertiary amine such as 1-methylimidazole) and the like, and an organic base of a metal carboxylate (e.g. an alkali metal or alkaline earth metal acetate such as sodium acetate and calcium acetate). These base catalysts are used alone as one kind or in combination of two or more kinds. Among them, metal alkoxides, metal hydroxides, and amines are preferable from the viewpoint of production, and it is preferable to use sodium hydroxide from the viewpoint of production such as easy availability and handleability. The amount of the base catalyst used can be arbitrarily set according to, for example, the kind of the raw materials used and the catalyst used and moreover the reaction conditions and is not particularly limited, but is preferably 0.01 to 100 parts by mass based on 100 parts by mass of the reaction raw materials.

Upon the above reaction, a reaction solvent may be used. Examples of the reaction solvent include, but not particularly limited to, water, methanol, ethanol, propanol, butanol, tetrahydrofuran, dioxane, 1-methoxy-2-propanol, ethylene glycol dimethyl ether, and ethylene glycol diethyl ether. These solvents are used alone as one kind or in combination of two or more kinds.

The amount of the solvent used can be arbitrarily set according to, for example, the kind of the raw materials used and the catalyst used and moreover the reaction conditions and is not particularly limited, but is preferably in the range of 0 to 2000 parts by mass based on 100 parts by mass of the reaction raw materials. Furthermore, the reaction temperature in the above reaction can be arbitrarily selected according to the reactivity of the reaction raw materials and is not particularly limited, but is usually within the range of 10 to 200° C.

In order to obtain compound (1) of the present embodiment, a higher reaction temperature is preferable. Specifically, the range of 60 to 200° C. is preferable. Although the reaction method is not particularly limited, for example, the raw materials (reactants) and the catalyst may be fed in a batch, or the raw materials (reactants) may be dripped successively in the presence of the catalyst. After the polycondensation reaction terminates, isolation of the obtained compound can be carried out according to a conventional method, and is not particularly limited. For example, by adopting a commonly used approach in which the temperature of the reaction vessel is elevated to 130 to 230° C. in order to remove unreacted raw materials, catalyst, etc. present in the system, and volatile portions are removed at about 1 to 50 mmHg, the compound that is the target compound can be obtained.

Examples of the preferable reaction conditions include conditions under which the reaction proceeds by using 1.0 mol to an excess amount of the above compound (1-x) and the above compound (1-y) based on 1 mol of the aldehyde or the ketone represented by the above formula (z1) or (z2), further using 0.001 to 1 mol of the acid catalyst, and reacting them at 50 to 150° C. at normal pressure for about 20 minutes to 100 hours.

The target compound can be isolated by a publicly known method after the reaction terminates. Compound (1) which is the target compound can be obtained by, for example, concentrating the reaction liquid, precipitating the reaction product by the addition of pure water, cooling the reaction liquid to room temperature, then separating the precipitates by filtration, filtering and drying the obtained solid matter, then performing separation from by-products and purification by column chromatography, and distilling off the solvent, followed by filtration and drying.

### [Resin (2)]

A resin of the present embodiment contains a constituent unit derived from the compound represented by the above formula (1). That is, the resin of the present embodiment contains the compound represented by the above formula (1) as a monomer component. The resin of the present embodiment is preferably a resin having a structure represented by formula (2) (hereinafter, also simply referred to as “resin (2)”).

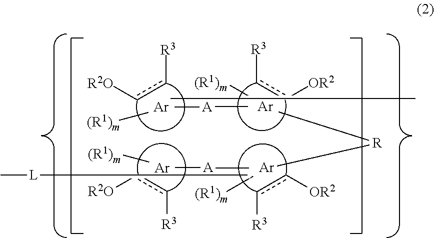

In formula (2) A, R, R¹ to R³, m, n, and p are each as defined in the above formula (1); and

L is a single bond or a linking group.

The resin of the present embodiment is more preferably a resin having a structure represented by formula (2-1).

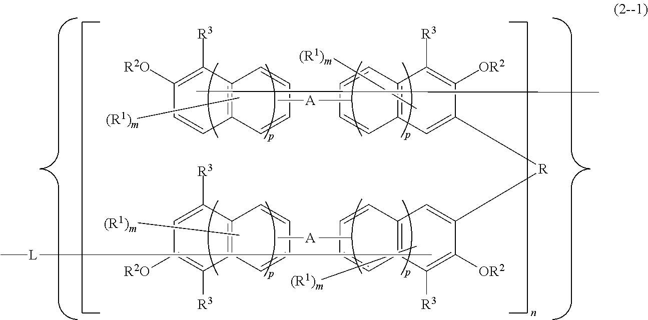

(In formula (2-1), A, R, R¹ to R³, m, n, and p are each as defined in the above formula (1); and

L is a single bond or a linking group.)

Examples of the above linking group include a residue derived from the crosslinkable compound, which will be mentioned later.

Preferable examples of L include a divalent hydrocarbon group having 1 to 30 carbon atoms.

Examples of the divalent hydrocarbon group include, but not particularly limited to, a linear or branched hydrocarbon group or a cyclic hydrocarbon group, such as an alkylene group.

In addition, the above resin (2) is preferably a resin represented by the following formula (2a) (hereinafter, also referred to as “resin (2a)”) from the viewpoint of more significantly obtaining the effect of the present invention.

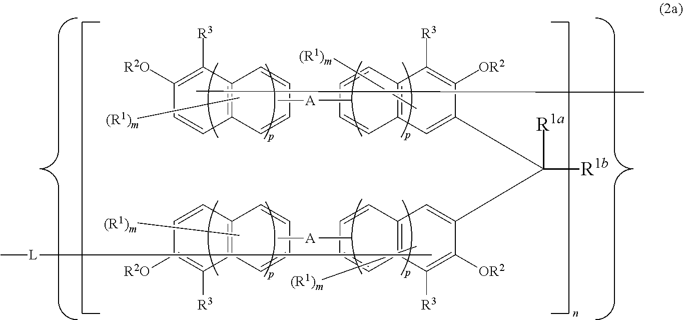

In addition, the above resin (2) is preferably a resin represented by the following formula (2b) (hereinafter, also simply referred to as “resin (2b)”) from the viewpoint of more significantly obtaining the effect of the present invention.

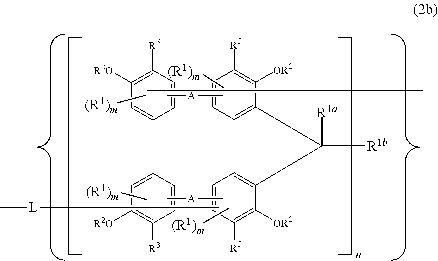

(2b)

In addition, the above resin (2b) is preferably a resin represented by the following formula (2c) (hereinafter, also simply referred to as “resin (2c)”) from the viewpoint of more significantly obtaining the effect of the present invention.

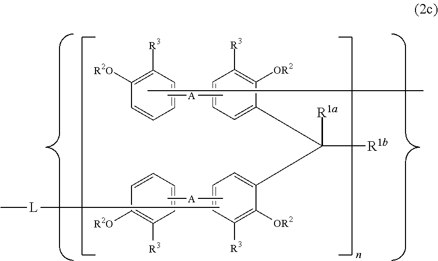

Resin (2) of the present embodiment is preferably a resin represented by the following formula (2d-1) (hereinafter, also referred to as “resin (2d-1)”) from the viewpoint of more significantly obtaining the effect of the present invention.

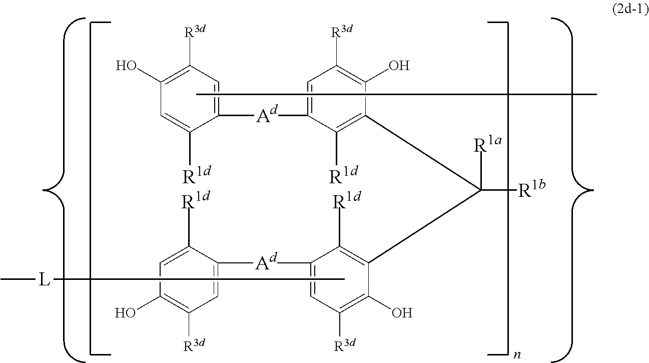

Resin (2) of the present embodiment is preferably a resin represented by the following formula (2d-1a) (hereinafter, also referred to as “resin (2d-1a)”) from the viewpoint of more significantly obtaining the effect of the present invention.

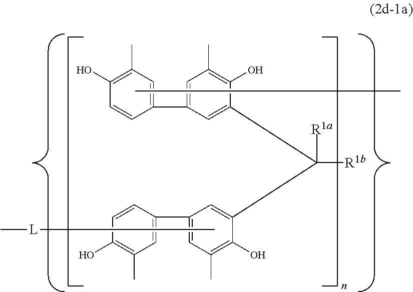

Resin (2) of the present embodiment is preferably a resin represented by the following formula (2d-2) (hereinafter, also referred to as “resin (2d-2)”) from the viewpoint of more significantly obtaining the effect of the present invention.

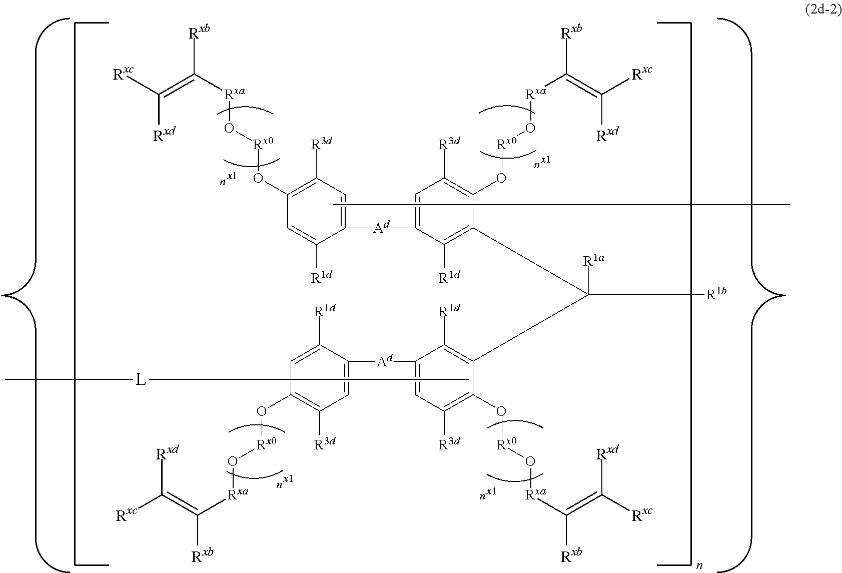

Resin (2) of the present embodiment is preferably a resin represented by the following formula (2d-3) (hereinafter, also referred to as “resin (2d-3)”) from the viewpoint of more significantly obtaining the effect of the present invention.

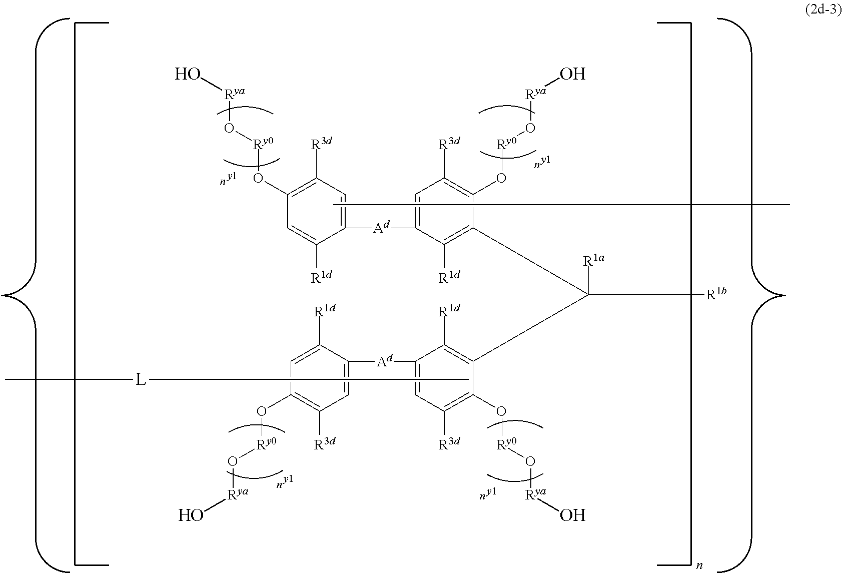

Resin (2) of the present embodiment is preferably a resin represented by the following formula (2d-4) (hereinafter, also referred to as “resin (2d-4)”) from the viewpoint of more significantly obtaining the effect of the present invention.

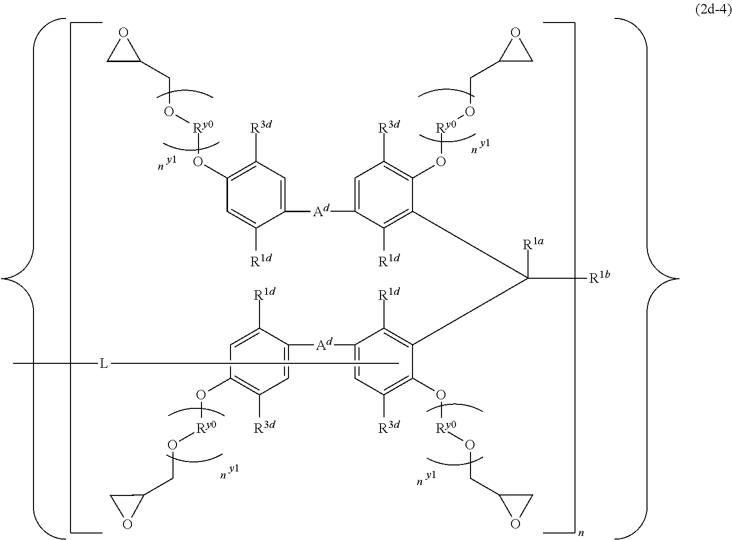

Resin (2) of the present embodiment is preferably a resin represented by the following formula (2d-5) (hereinafter, also referred to as “resin (2d-5)”) from the viewpoint of more significantly obtaining the effect of the present invention.

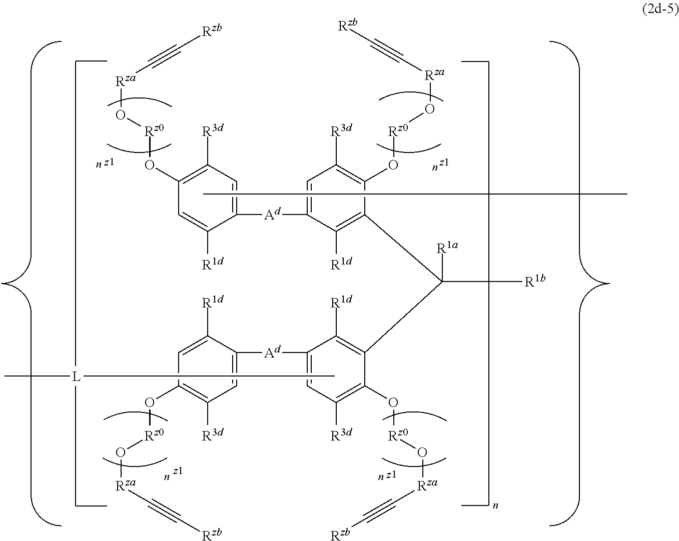

Resin (2) of the present embodiment is preferably a resin represented by the following formula (2d-6) (hereinafter, also referred to as “resin (2d-6)”) from the viewpoint of more significantly obtaining the effect of the present invention.

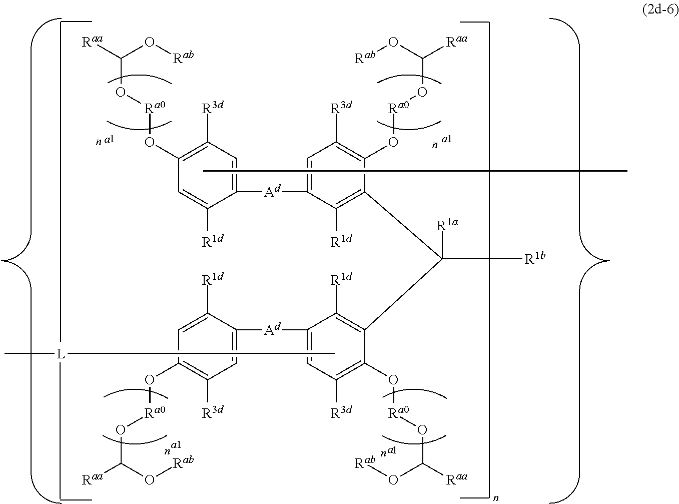

Resin (2) of the present embodiment is preferably a resin represented by the following formula (2d-7) (hereinafter, also referred to as “resin (2d-7)”) from the viewpoint of more significantly obtaining the effect of the present invention.

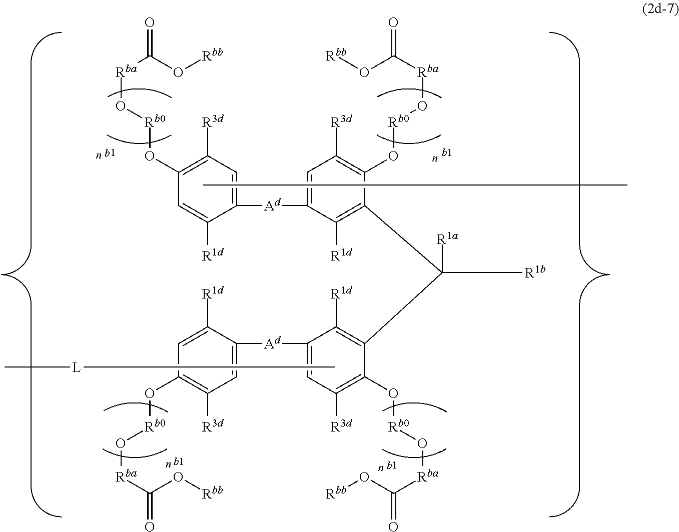

Resin (2) of the present embodiment is obtained by reacting the above compound (1) with a crosslinkable compound.

The crosslinkable compound may be any compound as long as it can oligomerize or polymerize the above compound (1), and examples thereof include an aldehyde, a ketone, a carboxylic acid, a carboxylic acid halide, a halogen-containing compound, an amino compound, an imino compound, an isocyanate compound, and an unsaturated hydrocarbon group-containing compound.

Examples of resin (2) of the present embodiment include, but not particularly limited to, a resin that has been made novolac obtained through, for example, a condensation reaction between the above compound (1) and an aldehyde or ketone, which is a crosslinkable compound.

Here, examples of the aldehyde used to make the above compound (1) novolac are the same as those for compound (z1) or its precursor used to synthesize compound (1) described above, but are not particularly limited thereto. These aldehydes are used alone as one kind or in combination of two or more kinds. In addition to these aldehydes, one or more ketones can be also used in combination. Among them, it is preferable to use one or more selected from the group consisting of benzaldehyde, phenylacetaldehyde, phenylpropylaldehyde, hydroxybenzaldehyde, chlorobenzaldehyde, nitrobenzaldehyde, methylbenzaldehyde, ethylbenzaldehyde, butylbenzaldehyde, cyclohexylbenzaldehyde, biphenylaldehyde, naphthaldehyde, anthracenecarbaldehyde, phenanthrenecarbaldehyde, pyrenecarbaldehyde, and furfural from the viewpoint of enabling exhibition of high heat resistance; it is preferable to use one or more selected from the group consisting of benzaldehyde, hydroxybenzaldehyde, chlorobenzaldehyde, nitrobenzaldehyde, methylbenzaldehyde, ethylbenzaldehyde, butylbenzaldehyde, cyclohexylbenzaldehyde, biphenylaldehyde, naphthaldehyde, anthracenecarbaldehyde, phenanthrenecarbaldehyde, pyrenecarbaldehyde, and furfural from the viewpoint of improving etching resistance; and it is more preferable to use formaldehyde. The amount of the aldehyde used is not particularly limited, but is preferably 0.2 to 5 mol and more preferably 0.5 to 2 mol based on 1 mol of the above compound (1).

Examples of the ketone used to make the above compound (1) novolac are the same as those for compound (z2) used to synthesize compound (1) described above, but are not particularly limited thereto. These ketones are used alone as one kind or in combination of two or more kinds. Among them, it is preferable to use one or more selected from the group consisting of cyclopentanone, cyclohexanone, norbornanone, tricyclohexanone, tricyclodecanone, adamantanone, fluorenone, benzofluorenone, acenaphthenequinone, acenaphthenone, anthraquinone, acetophenone, diacetylbenzene, triacetylbenzene, acetonaphthone, diphenylcarbonylnaphthalene, phenylcarbonylbiphenyl, diphenylcarbonylbiphenyl, benzophenone, diphenylcarbonylbenzene, triphenylcarbonylbenzene, benzonaphthone, diphenylcarbonylnaphthalene, phenylcarbonylbiphenyl, and diphenylcarbonylbiphenyl from the viewpoint of enabling exhibition of high heat resistance, and it is more preferable to use one or more selected from the group consisting of acetophenone, diacetylbenzene, triacetylbenzene, acetonaphthone, diphenylcarbonylnaphthalene, phenylcarbonylbiphenyl, diphenylcarbonylbiphenyl, benzophenone, diphenylcarbonylbenzene, triphenylcarbonylbenzene, benzonaphthone, diphenylcarbonylnaphthalene, phenylcarbonylbiphenyl, and diphenylcarbonylbiphenyl from the viewpoint of improving etching resistance. The amount of the ketone used is not particularly limited, but is preferably 0.2 to 5 mol and more preferably 0.5 to 2 mol based on 1 mol of the above compound (1).

A catalyst can also be used in the condensation reaction between the above compound (1) and the aldehyde or ketone. The acid catalyst or base catalyst to be used herein can be arbitrarily selected for use from publicly known catalysts and is not particularly limited. Examples of such acid catalysts and base catalysts are the same as those described for the method for producing the above compound (1). These catalysts are used alone as one kind or in combination of two or more kinds. Among them, organic acids and solid acids are preferable from the viewpoint of production, and hydrochloric acid or sulfuric acid is preferable from the viewpoint of production such as easy availability and handleability. The amount of the acid catalyst used can be arbitrarily set according to, for example, the kind of the raw materials used and the catalyst used and moreover the reaction conditions and is not particularly limited, but is preferably 0.01 to 100 parts by mass based on 100 parts by mass of the reaction raw materials.

However, in the case of a copolymerization reaction with a compound having a non-conjugated double bond, such as indene, hydroxyindene, benzofuran, hydroxyanthracene, acenaphthylene, biphenyl, bisphenol, trisphenol, dicyclopentadiene, tetrahydroindene, 4-vinylcyclohexene, norbornadiene, 5-vinylnorborn-2-ene, α-pinene, β-pinene, and limonene, the aldehyde or ketone is not necessarily needed.

A reaction solvent can also be used in the condensation reaction between the above compound (1) and the aldehyde or ketone. The reaction solvent in this polycondensation can be arbitrarily selected for use from publicly known solvents and is not particularly limited, and examples thereof include water, methanol, ethanol, propanol, butanol, 1-methoxy-2-propanol, tetrahydrofuran, dioxane, and a mixed solvent thereof. These solvents are used alone as one kind or in combination of two or more kinds.

The amount of the solvent used can be arbitrarily set according to, for example, the kind of the raw materials used and the catalyst used and moreover the reaction conditions and is not particularly limited, but is preferably in the range of 0 to 2000 parts by mass based on 100 parts by mass of the reaction raw materials. Furthermore, the reaction temperature can be arbitrarily selected according to the reactivity of the reaction raw materials and is not particularly limited, but is usually within the range of 10 to 200° C. Examples of the reaction method include a method in which the above compound (1), the aldehyde and/or ketone, and the catalyst are fed in a batch, and a method in which the above compound (1) and the aldehyde and/or ketone are dripped successively in the presence of the catalyst.

After the polycondensation reaction terminates, isolation of the obtained resin can be carried out according to a conventional method, and is not particularly limited. For example, by adopting a commonly used approach in which the temperature of the reaction vessel is elevated to 130 to 230° C. in order to remove unreacted raw materials, catalyst, etc. present in the system, and volatile portions are removed at about 1 to 50 mmHg, the target compound (for example, the resin that has been made novolac) can be obtained.

Resin (2) of the present embodiment is obtained upon the synthesis reaction of the above compound (1). This corresponds to the case where the same aldehyde or ketone is used upon polymerizing the above compound (1) as that used in the synthesis of the above compound (1).

Herein, resin (2) of the present embodiment may be a homopolymer of the above compound (1), or may be a copolymer with an additional phenol. Here, examples of the copolymerizable phenol include, but not particularly limited to, phenol, cresol, dimethylphenol, trimethylphenol, butylphenol, phenylphenol, diphenylphenol, naphthylphenol, resorcinol, methylresorcinol, catechol, butylcatechol, methoxyphenol, propylphenol, pyrogallol, and thymol.

In addition, resin (2) of the present embodiment may be a copolymer with a polymerizable monomer other than the additional phenol mentioned above. Examples of the copolymerization monomer include, but not particularly limited to, naphthol, methylnaphthol, methoxynaphthol, dihydroxynaphthalene, indene, hydroxyindene, benzofuran, hydroxyanthracene, acenaphthylene, biphenyl, bisphenol, trisphenol, dicyclopentadiene, tetrahydroindene, 4-vinylcyclohexene, norbornadiene, vinylnorbornaene, pinene, and limonene. The resin of the present embodiment may be a copolymer of two or more components (for example, a binary to quaternary system) composed of the above compound (1) and the above phenol, may be a copolymer of two or more components (for example, a binary to quaternary system) composed of the above compound (1) and the above copolymerization monomer, or may be a copolymer of three or more components (for example, a tertiary to quaternary system) composed of the above compound (1), the above phenol, and the above copolymerization monomer.

The weight average molecular weight (Mw) of resin (2) of the present embodiment is not particularly limited, and is, in terms of polystyrene through GPC measurement, preferably 300 to 100,000, more preferably 500 to 30,000, and still more preferably 750 to 20,000. In addition, the dispersity (weight average molecular weight Mw/number average molecular weight Mn) of the resin of the present embodiment is preferably in the range of 1 to 7 from the viewpoint of enhancing crosslinking efficiency while suppressing volatile components during baking.

Compound (1) and/or resin (2) described above preferably have high solubility in a solvent from the viewpoint of easier application to a wet process, etc. More specifically, in the case of using propylene glycol monomethyl ether (hereinafter, also referred to as “PGME”) and/or propylene glycol monomethyl ether acetate (hereinafter, also referred to as “PGMEA”) as a solvent, it is preferable that compound (1) and/or resin (2) have a solubility of 10% by mass or more in the solvent. Here, the solubility in PGME and/or PGMEA is defined as “mass of compound (1) and/or resin (2)/(mass of compound (1) and/or resin (2)+mass of solvent)×100 (% by mass).” For example, in the case where the solubility of compound (1) and/or resin (2) in PGMEA is “10% by mass or more,” the solubility of 10 g of the above compound (1) and/or resin (2) in 90 g of PGMEA is evaluated as high, and in the case where said solubility is “less than 10% by mass,” the solubility is evaluated as not high.

### [Composition]

A composition of the present embodiment contains compound (1) and/or resin (2).

Since the composition of the present embodiment contains compound (1) and/or resin (2) of the present embodiment, a wet process can be applied, and heat resistance and flattening properties are excellent. Furthermore, since the composition of the present embodiment contains compound (1) and/or resin (2), film deterioration during high temperature baking is suppressed, and a film for lithography excellent in etching resistance against oxygen plasma etching or the like can be formed. Furthermore, the composition of the present embodiment is also excellent in adhesiveness to a resist film and can therefore form an excellent resist pattern. Therefore, the composition of the present embodiment is preferably used for film formation for lithography.

In addition, the composition of the present embodiment can also form a resist film.

The composition of the present embodiment has high refractive index ascribable to its high aromatic ring density and is prevented from being stained by heat treatment in a wide range from a low temperature to a high temperature. Therefore, the composition of the present embodiment is preferably used also for optical component formation.

In the present embodiment, the film for lithography refers to a film having a larger dry etching rate relative to photoresist films. Examples of the above film for lithography include a film for being embedded to steps of a layer to be processed and flattening the layer, a resist upper layer film, and a resist underlayer film.

### [Composition for Film Formation for Lithography]

The film forming composition for lithography of the present embodiment may contain a solvent, a crosslinking agent, a crosslinking promoting agent, an acid generating agent, a basic compound, and a further component, in addition to compound (1) and/or resin (2) of the present embodiment, if required. Hereinafter, these optional components will be described.

### [Solvent]

The film forming composition for lithography of the present embodiment may contain a solvent. The solvent is not particularly limited as long as it is a solvent that can dissolve compound (1) and/or resin (2) of the present embodiment. Here, compound (1) and/or resin (2) of the present embodiment has excellent solubility in an organic solvent, as mentioned above, and therefore, various organic solvents are suitably used.

Examples of the solvent include, but not particularly limited to, a ketone-based solvent such as acetone, methyl ethyl ketone, methyl isobutyl ketone, and cyclohexanone; a cellosolve-based solvent such as PGME and PGMEA; an ester-based solvent such as ethyl lactate, methyl acetate, ethyl acetate, butyl acetate, isoamyl acetate, ethyl lactate, methyl methoxypropionate, and methyl hydroxyisobutyrate; an alcohol-based solvent such as methanol, ethanol, isopropanol, and 1-ethoxy-2-propanol; and an aromatic hydrocarbon such as toluene, xylene, and anisole. These solvents are used alone as one kind or in combination of two or more kinds.

Among the above solvents, from the viewpoint of safety, one or more selected from the group consisting of cyclohexanone, PGME, PGMEA, ethyl lactate, methyl hydroxyisobutyrate, and anisole are preferable.

In the composition of the present embodiment, the solid content is preferably 1 to 80% by mass, more preferably 1 to 50% by mass, further preferably 2 to 40% by mass, and still more preferably 2 to 10% by mass with 90 to 98% by mass of the solvent based on 100% by mass of the total mass of the solid content and the solvent but not particularly limited thereto.

In the composition of the present embodiment, the amount of the solvent is preferably 20 to 99% by mass, more preferably 50 to 99% by mass, further preferably 60 to 98% by mass, and still more preferably 90 to 98, by mass based on 100% by mass of the total mass of the solid components and the solvent but not particularly limited thereto. In the present specification, “the solid components” refer to components except for the solvent.

The content of the solvent is not particularly limited and is preferably 100 to 10,000 parts by mass, more preferably 200 to 5,000 parts by mass, and still more preferably 200 to 1,000 parts by mass, based on 100 parts by mass of compound (1) and/or resin (2) of the present embodiment, from the viewpoint of solubility and film formation.

### [Crosslinking Agent]

The film forming composition for lithography of the present embodiment may contain a crosslinking agent from the viewpoint of, for example, suppressing intermixing. The crosslinking agent is not particularly limited, but those described in, for example, International Publication No. WO 2013/024779 and International Publication No. WO 2018/016614 can be used.

Examples of the crosslinking agent include, but not particularly limited to, a phenol compound, an epoxy compound, a cyanate compound, an amino compound, a benzoxazine compound, an acrylate compound, a melamine compound, a guanamine compound, a glycoluril compound, a urea compound, an isocyanate compound, and an azide compound. These crosslinking agents are used alone as one kind or in combination of two or more kinds. Among them, one or more selected from the group consisting of a benzoxazine compound, an epoxy compound, and a cyanate compound are preferable, and a benzoxazine compound is more preferable from the viewpoint of improving etching resistance.

In the present embodiment, the content of the crosslinking agent is not particularly limited and is preferably 0.1 to 100 parts by mass, more preferably 5 to 50 parts by mass, and still more preferably 10 to 40 parts by mass, based on 100 parts by mass of compound (1) and/or resin (2) of the present embodiment. By setting the content of the crosslinking agent to the above range, occurrence of a mixing event with a resist film tends to be prevented. Also, an antireflection effect is enhanced, and film formability after crosslinking tends to be enhanced.

### [Crosslinking Promoting Agent]

The film forming composition for lithography of the present embodiment may contain a crosslinking promoting agent for promoting crosslinking reaction (curing reaction), if required. Examples of the crosslinking promoting agent include a radical polymerization initiator.

The radical polymerization initiator may be a photopolymerization initiator that initiates radical polymerization by light, or may be a thermal polymerization initiator that initiates radical polymerization by heat. Examples of the radical polymerization initiator include, but not particularly limited to, a ketone-based photopolymerization initiator, an organic peroxide-based polymerization initiator, and an azo-based polymerization initiator.

The radical polymerization initiator is not particularly limited, but those described in, for example, International Publication No. WO 2018/016614 can be used.

These radical polymerization initiators are used alone as one kind or in combination of two or more kinds.

The content of the radical polymerization initiator in the present embodiment is not particularly limited, and is preferably 0.05 to 25 parts by mass and more preferably 0.1 to 10 parts by mass based on the compound or resin of the present embodiment as 100 parts by mass. When the content of the radical polymerization initiator is 0.05 parts by mass or more, there is a tendency to make it possible to prevent curing from being insufficient. On the other hand, when the content of the radical polymerization initiator is 25 parts by mass or less, there is a tendency to make it possible to prevent the long term storage stability at room temperature from being impaired.

### [Acid Generating Agent]

The film forming composition for lithography of the present embodiment may contain an acid generating agent from the viewpoint of, for example, further accelerating crosslinking reaction by heat. An acid generating agent that generates an acid by thermal decomposition, an acid generating agent that generates an acid by light irradiation, and the like are known, any of which can be used. The acid generating agent is not particularly limited, but those described in, for example, International Publication No. WO 2013/024779 can be used.

The content of the acid generating agent in the film forming composition for lithography is not particularly limited, and is preferably 0.1 to 50 parts by mass and more preferably 0.5 to 40 parts by mass, based on 100 parts by mass of compound (1) and/or resin (2) of the present embodiment. By setting the content of the acid generating agent to the above range, crosslinking reaction tends to be enhanced and occurrence of a mixing event with a resist film tends to be prevented.

### [Basic Compound]

The film forming composition for lithography of the present embodiment may also contain a basic compound from the viewpoint of, for example, improving storage stability.

The basic compound plays a role to prevent crosslinking reaction from proceeding due to a trace amount of an acid generated from the acid generating agent, that is, a role as a quencher against the acid. Examples of such a basic compound include, but not particularly limited to, those described in International Publication No. WO 2013/024779.

The content of the basic compound in the film forming composition for lithography of the present embodiment is not particularly limited, and is preferably 0.001 to 2 parts by mass and more preferably 0.01 to 1 part by mass, based on 100 parts by mass of compound (1) and/or resin (2) of the present embodiment. By setting the content of the basic compound to the above range, storage stability tends to be enhanced without excessively deteriorating crosslinking reaction.

### [Additional Additive]

The film forming composition for lithography of the present embodiment may also contain an additional resin and/or compound for the purpose of conferring thermosetting or light curing properties or controlling absorbance. Examples of such an additional resin and/or compound include, but not particularly limited to, a naphthol resin, a xylene resin, naphthol-modified resin, a phenol-modified resin of a naphthalene resin; a polyhydroxystyrene, a dicyclopentadiene resin, a (meth)acrylate, a dimethacrylate, a trimethacrylate, a tetramethacrylate, a naphthalene ring such as a vinylnaphthalene and a polyacenaphthylene, phenanthrenequinone, a biphenyl ring such as fluorene, a resin containing a heterocycle having a heteroatom such as thiophene and indene and a resin containing no aromatic ring; and a resin or compound containing an aliphatic structure such as a rosin-based resin, cyclodextrin, adamantane(poly)ol, tricyclodecane(poly)ol, and a derivative thereof. The film forming composition for lithography of the present embodiment may also contain a publicly known additive. Examples of the publicly known additive include, but not limited to, a thermal and/or light curing catalyst, a polymerization inhibitor, a flame retardant, a filler, a coupling agent, a thermosetting resin, a light curable resin, a dye, a pigment, a thickener, a lubricant, an antifoaming agent, a leveling agent, an ultraviolet absorber, a surfactant, a colorant, and a nonionic surfactant.

The underlayer film for lithography of the present embodiment is formed from the film forming composition for lithography of the present embodiment.

### [Composition for Resist Film Formation]

As described above, the composition of the present embodiment is preferably used for resist film formation in another aspect. That is, a resist film of the present embodiment includes the composition of the present embodiment. A film formed by applying the composition of the present embodiment can also be used with a resist pattern formed, if required.

The composition of the present embodiment can be used for the composition for film formation for lithography used for chemical amplification resists (hereinafter, also referred to as the “composition for resist film formation”).

In addition, the composition for resist film formation of the present embodiment preferably contains a solvent. Examples of the solvent can include, but not particularly limited to, ethylene glycol monoalkyl ether acetates such as ethylene glycol monomethyl ether acetate, ethylene glycol monoethyl ether acetate, ethylene glycol mono-n-propyl ether acetate, and ethylene glycol mono-n-butyl ether acetate; ethylene glycol monoalkyl ethers such as ethylene glycol monomethyl ether and ethylene glycol monoethyl ether; propylene glycol monoalkyl ether acetates such as PGMEA, propylene glycol monoethyl ether acetate, propylene glycol mono-n-propyl ether acetate, and propylene glycol mono-n-butyl ether acetate; propylene glycol monoalkyl ethers such as PGME and propylene glycol monoethyl ether; ester lactates such as methyl lactate, ethyl lactate, n-propyl lactate, n-butyl lactate, and n-amyl lactate; aliphatic carboxylic acid esters such as methyl acetate, ethyl acetate, n-propyl acetate, n-butyl acetate, n-amyl acetate, n-hexyl acetate, methyl propionate, and ethyl propionate; other esters such as methyl 3-methoxypropionate, ethyl 3-methoxypropionate, methyl 3-ethoxypropionate, ethyl 3-ethoxypropionate, methyl 3-methoxy-2-methylpropionate, 3-methoxybutylacetate, 3-methyl-3-methoxybutylacetate, butyl 3-methoxy-3-methylpropionate, butyl 3-methoxy-3-methylbutyrate, methyl acetoacetate, methyl pyruvate, and ethyl pyruvate; aromatic hydrocarbons such as toluene and xylene; ketones such as 2-heptanone, 3-heptanone, 4-heptanone, cyclopentanone (hereinafter, also referred to as “CPN”), and cyclohexanone (hereinafter, also referred to as “CHN”); amides such as N,N-dimethylformamide, N-methylacetamide, N,N-dimethylacetamide, and N-methylpyrrolidone; and lactones such as γ-lactone. These solvents are used alone as one kind or in combination of two or more kinds.

The solvent used in the present embodiment is preferably a safe solvent, more preferably one or more selected from PGMEA, PGME, CHN, CPN, 2-heptanone, anisole, butyl acetate, ethyl propionate, and ethyl lactate, and still more preferably one or more selected from PGMEA, PGME, and CHN.

In the composition for resist film formation of the present embodiment, the amount of the solid components is preferably 1 to 80% by mass, more preferably 1 to 50% by mass, further preferably 2 to 40% by mass, and still more preferably 2 to 10% by mass with 90 to 98% by mass of the solvent based on the total mass of the solid components and the solvent as 100% by mass but not particularly limited thereto.

In the composition for resist film formation of the present embodiment, the amount of the solvent is preferably 20 to 99% by mass, more preferably 50 to 99% by mass, further preferably 60 to 98% by mass, and still more preferably 90 to 98% by mass based on the total mass of the solid components and the solvent as 100% by mass but not particularly limited thereto.

The composition for resist film formation of the present embodiment may contain one or more selected from the group consisting of an acid generating agent, an acid crosslinking agent, an acid diffusion controlling agent, and an additional component, as other solid components of compound (1) and/or resin (2) of the present embodiment.

Herein, as the acid generating agent, the acid crosslinking agent, the acid diffusion controlling agent, and the additional component, publicly known agents can be used, and they are not particularly limited, but those described in International Publication No. WO 2013/024778 are preferable, for example.

In the composition for resist film formation of the present embodiment, the content of compound (1) and/or resin (2) of the present embodiment used as a base material for a resist is preferably 1 to 100%, more preferably 50 to 99.4% by mass, still more preferably 55 to 90% by mass, further preferably 60 to 80% by mass, and particularly preferably 60 to 70% by mass based on the total mass of the solid components but not particularly limited thereto. When the content of compound (1) and/or resin (2) falls within the above range, there is a tendency to further improve resolution, and line edge roughness (hereinafter, also referred to as “LER”) is further decreased.

When both of compound (1) and resin (2) are contained, the above content refers to the total amount of these components.

The composition for resist film formation of the present embodiment, if required, may contain various additives such as a dissolution promoting agent, dissolution controlling agent, sensitizing agent, surfactant, organic carboxylic acid or oxo acid of phosphor or a derivative thereof, thermosetting catalyst, light curing catalyst, polymerization inhibitor, flame retardant, filler, coupling agent, thermosetting resin, light curable resin, dye, pigment, thickener, lubricant, antifoaming agent, leveling agent, ultraviolet absorber, surfactant such as a nonionic surfactant, and colorant within the range not inhibiting the objects of the present embodiment.

These additives are used alone as one kind or in combination of two or more kinds.

In the composition for resist film formation of the present embodiment, contents of compound (1) and/or resin (2) of the present embodiment, the acid generating agent, the acid crosslinking agent, the acid diffusion controlling agent, and the additional component (compound (1) and resin (2)/acid generating agent/acid crosslinking agent/acid diffusion controlling agent/additional component) are, in terms of a by mass base on solid,
preferably 1 to 100/0 to 49/0 to 49/0 to 49/0 to 99,
more preferably 50 to 99.4/0.001 to 49/0.5 to 49/0.001 to 49/0 to 49,
further preferably 55 to 90/1 to 40/0.5 to 40/0.01 to 10/0 to 5,
still more preferably 60 to 80/3 to 30/1 to 30/0.01 to 5/0 to 1, and
particularly preferably 60 to 70/10 to 25/2 to 20/0.01 to 3/0.

The content ratio of each component is selected from each range so that the summation thereof is 100% by mass. When the content ratio of each component falls within the above range, performance such as sensitivity, resolution, and developability tends to be excellent.

The composition for resist film formation of the present embodiment is generally prepared by dissolving each component in a solvent upon use into a homogeneous solution, and then if required, filtering through a filter or the like with a pore diameter of about 0.2 μm, for example.

The composition for resist film formation of the present embodiment can contain an additional resin other than the resin of the present embodiment, within the range not inhibiting the objects of the present invention. Examples of the additional resin include, but not particularly limited to, a novolac resin, a polyvinyl phenol, a polyacrylic acid, an epoxy resin, a polyvinyl alcohol, a styrene-maleic anhydride resin, and an addition polymerization resin.

Examples of the addition polymerization resin include, but not particularly limited to, an acrylic acid, a vinyl alcohol, a vinyl phenol, and a polymer including a maleimide compound as a monomeric unit, and derivatives thereof.

The content of the additional resin is not particularly limited and is appropriately adjusted according to the kind of compound (1) and/or resin (2) of the present embodiment used, but is preferably 30 parts by mass or less, more preferably 10 parts by mass or less, further preferably 5 parts by mass or less, and still more preferably 0 parts by mass based on 100 parts by mass of compound (1) and/or resin (2) of the present embodiment.

The composition for resist film formation of the present embodiment can be used to form an amorphous film by spin coating. Also, the composition for resist film formation of the present embodiment can be applied to a general semiconductor production process. Any of positive type and negative type resist patterns can be individually prepared depending on the type of compound (1) and/or resin (2) of the present embodiment, and the kind of a developing solution to be used.

In the case of a positive type resist pattern, the dissolution rate of the amorphous film formed by spin coating with the composition for resist film formation of the present embodiment in a developing solution at 23° C. is preferably 5 angstrom/sec or less, more preferably 0.05 to 5 angstrom/sec, and further preferably 0.0005 to 5 angstrom/sec. When the dissolution rate is 5 angstrom/sec or less, there is a tendency of the amorphous film to be insoluble in a developing solution and therefore to easily form a resist. When the dissolution rate is 0.0005 angstrom/sec or more, the resolution may improve. It is presumed that this is because due to the change in the solubility before and after exposure of compound (1) and/or resin (2) of the present embodiment, contrast at the interface between the exposed portion being dissolved in a developing solution and the unexposed portion not being dissolved in a developing solution is increased. Also, effects of reducing LER and defects are seen.

In the case of a negative type resist pattern, the dissolution rate of the amorphous film formed by spin coating with the composition for resist film formation of the present embodiment in a developing solution at 23° C. is preferably 10 angstrom/sec or more. When the dissolution rate is 10 angstrom/sec or more, the amorphous film more easily dissolves in a developing solution, and is suitable for a resist. When the dissolution rate is 10 angstrom/sec or more, the resolution may improve. It is presumed that this is because the micro surface portion of compound (1) and/or resin (2) of the present embodiment dissolves, and LER is reduced. Also, effects of reducing defects are seen.

The above dissolution rate can be determined by immersing the amorphous film in a developing solution for a predetermined period of time at 23° C. and then measuring the film thickness before and after immersion by a publicly known method such as visual, ellipsometric, or QCM method.

In the case of a positive type resist pattern, the dissolution rate of the portion exposed by radiation such as KrF excimer laser, extreme ultraviolet, electron beam, or X-ray, of the amorphous film formed by spin coating with the composition for resist film formation of the present embodiment, in a developing solution at 23° C. is preferably 10 angstrom/sec or more. When the dissolution rate is 10 angstrom/sec or more, the above portion more easily dissolves in a developing solution, and is suitable for a resist. When the dissolution rate is 10 angstrom/sec or more, the resolution may improve. It is presumed that this is because the micro surface portion of compound (1) and/or resin (2) of the present embodiment dissolves, and LER is reduced. Also, effects of reducing defects are seen.

In the case of a negative type resist pattern, the dissolution rate of the portion exposed by radiation such as KrF excimer laser, extreme ultraviolet, electron beam, or X-ray, of the amorphous film formed by spin coating with the composition for resist film formation of the present embodiment, in a developing solution at 23° C. is preferably 5 angstrom/sec or less, more preferably 0.05 to 5 angstrom/sec, and further preferably 0.0005 to 5 angstrom/sec. When the dissolution rate is 5 angstrom/sec or less, there is a tendency of the above portion to be insoluble in a developing solution and therefore to easily form a resist. When the dissolution rate is 0.0005 angstrom/sec or more, the resolution may improve. It is presumed that this is because due to the change in the solubility before and after exposure of compound (1) and/or resin (2) of the present embodiment, contrast at the interface between the unexposed portion being dissolved in a developing solution and the exposed portion not being dissolved in a developing solution is increased. Also, effects of reducing LER and defects are seen.

Component (1) and/or resin (2) to be contained in the composition for resist film formation of the present embodiment dissolves at preferably 1% by mass or more, more preferably 5% by mass or more, and still more preferably 10% by mass or more at 23° C. in a solvent that is selected from the group consisting of PGMEA, PGME, CHN, CPN, 2-heptanone, anisole, butyl acetate, ethyl propionate, and ethyl lactate and exhibits the highest ability to dissolve component (1) and/or resin (2).

Component (1) and/or resin (2) to be contained in the composition for resist film formation of the present embodiment dissolves at preferably 20% by mass or more at 23° C. in a solvent that is selected from the group consisting of PGMEA, PGME, and CHN and exhibits the highest ability to dissolve the component (A). Component (1) and/or resin (2) to be contained in the composition for resist film formation of the present embodiment dissolves at preferably 20% by mass or more at 23° C. in PGMEA. When the above conditions are met, the composition is easily used in a semiconductor production process at a full production scale.

The composition for resist film formation of the present embodiment may contain an additional resin other than the resin of the present embodiment within the range not inhibiting the objects of the present invention. Examples of such an additional resin include a novolac resin, a polyvinyl phenol, a polyacrylic acid, a polyvinyl alcohol, a styrene-maleic anhydride resin, and a polymer containing an acrylic acid, vinyl alcohol, or vinylphenol as a monomeric unit, and derivatives thereof. The content of the additional resin is appropriately adjusted according to the kind of compound (1) and/or resin (2) of the present embodiment used, but is preferably 30 parts by mass or less, more preferably 10 parts by mass or less, still more preferably 5 parts by mass or less, and especially preferably 0 parts by mass based on 100 parts by mass of compound (1) and/or resin (2) of the present embodiment.

In addition, the composition for resist film formation of the present embodiment may contain the crosslinking agent, crosslinking promoting agent, radical polymerization initiator, acid generating agent, and basic compound listed in the section of [Composition] mentioned above, within the range not inhibiting the objects of the present invention.

### [Resist Permanent Film]

It is more preferable to use the composition of the present embodiment for forming a resist permanent film that remains in a final product, if required, with a resist pattern formed. That is, a resist permanent film of the present embodiment includes the composition of the present embodiment. A film formed by applying the composition of the present embodiment is suitable as a resist permanent film that remains also in a final product, if required, after formation of a resist pattern. Specific examples of the permanent film include, in relation to semiconductor devices, a solder resist, a package material, an underfill material, a package adhesive layer for circuit elements and the like, and an adhesive layer between integrated circuit elements and circuit substrates, and in relation to thin displays, a thin film transistor protecting film, a liquid crystal color filter protecting film, a black matrix, and a spacer. Particularly, the resist permanent film containing the composition of the present embodiment is excellent in heat resistance and humidity resistance and furthermore, also has the excellent advantage that contamination by sublimable components is reduced. Particularly, for a display material, a material that achieves all of high sensitivity, high heat resistance, and hygroscopic reliability with reduced deterioration in image quality due to significant contamination can be obtained.

In the case of using the composition of the present embodiment for resist permanent film purposes, a curing agent as well as, if required, various additives such as an additional resin, a surfactant, a dye, a filler, a crosslinking agent, and a dissolution promoting agent can be added and dissolved in an organic solvent to prepare a composition for resist permanent films.

### [Resist Pattern Formation Method]

A resist pattern formation method of the present embodiment preferably includes: an underlayer film formation step of forming an underlayer film on a substrate using the composition of the present embodiment; a photoresist film formation step of forming at least one photoresist film on the underlayer film formed through the underlayer film formation step; and a step of irradiating a predetermined region of the photoresist film formed through the photoresist film formation step with radiation for development. The resist pattern formation method of the present embodiment can be used for forming various patterns, and is preferably a method for forming an insulating film pattern.

The resist pattern formation method of the present embodiment preferably includes a photoresist film formation step of forming a photoresist film on the substrate using the composition of the present embodiment; and a step of irradiating a predetermined region of the photoresist film formed through the photoresist film formation step with radiation for development. The resist pattern formation method of the present embodiment can be used for forming various patterns, and is preferably a method for forming an insulating film pattern.

### [Circuit Pattern Formation Method]

The circuit pattern formation method of the present embodiment includes: an underlayer film formation step of forming an underlayer film on a substrate using the composition of the present embodiment; an intermediate layer film formation step of forming an intermediate layer film on the underlayer film formed through the underlayer film formation step; a photoresist film formation step of forming at least one photoresist film on the intermediate layer film formed through the intermediate layer film formation step; a resist pattern formation step of irradiating a predetermined region of the photoresist film formed through the photoresist film formation step with radiation for development, to form a resist pattern; an intermediate layer film pattern formation step of etching the intermediate layer film with the resist pattern formed through the resist pattern formation step as a mask, to form an intermediate layer film pattern; an underlayer film pattern formation step of etching the underlayer film with the intermediate layer film pattern formed through the intermediate layer film pattern formation step as a mask, thereby forming an underlayer film pattern; and a substrate pattern formation step of etching the substrate with the underlayer film pattern formed through the underlayer film pattern formation step as a mask, thereby forming a pattern on the substrate.

The underlayer film for lithography of the present embodiment is formed from the film forming composition for lithography of the present embodiment. The formation method is not particularly limited and a publicly known method can be applied. The underlayer film can be formed by, for example, applying the film forming composition for lithography of the present embodiment onto a substrate by a publicly known coating method, printing method, or the like such as spin coating, screen printing, or the like and then removing an organic solvent by volatilization or the like.

It is preferable to perform baking in the formation of the underlayer film, for preventing occurrence of a mixing event with a resist upper layer film while accelerating crosslinking reaction. In this case, the baking temperature is not particularly limited and is preferably in the range of 80 to 450° C., and more preferably 200 to 400° C. The baking time is not particularly limited and is preferably in the range of 10 to 300 seconds. The thickness of the underlayer film can be arbitrarily selected according to required performance and is not particularly limited, but is preferably 30 to 20,000 nm, and more preferably 50 to 15,000 nm.

After preparing the underlayer film, it is preferable to prepare a silicon-containing resist film or a single-layer resist made of a hydrocarbon on the underlayer film in the case of a two-layer process, and to prepare a silicon-containing intermediate layer on the underlayer film and further prepare a silicon-free single-layer resist film on the silicon-containing intermediate layer in the case of a three-layer process. In this case, for a photoresist material for forming this resist film, a publicly known material can be used.

As a silicon-containing resist material for a two-layer process, a positive type resist material which is obtained by using, as a base polymer, a silicon atom-containing polymer such as a polysilsesquioxane derivative or vinylsilane derivative and which further includes an organic solvent and a generating agent, and, if required, basic compound and the like is preferably used from the viewpoint of etching resistance. Here, a publicly known polymer that is used in this kind of resist material can be used as the silicon atom-containing polymer.

A polysilsesquioxane-based intermediate layer is preferably used as the silicon-containing intermediate layer for a three-layer process. By imparting effects as an antireflection film to the intermediate layer, there is a tendency to make it possible to effectively suppress reflection. For example, use of a material containing a large amount of an aromatic group and having high substrate etching resistance as the underlayer film in a process for exposure at 193 nm tends to increase a k value and enhance substrate reflection. However, the intermediate layer suppresses the reflection so that the substrate reflection can be 0.5% or less. The intermediate layer having such an antireflection effect is not limited, and polysilsesquioxane that crosslinks by an acid or heat in which a light absorbing group having a phenyl group or a silicon-silicon bond is introduced is preferably used for exposure at 193 nm.

Alternatively, an intermediate layer formed by chemical vapor deposition (CVD) may be used. The intermediate layer highly effective as an antireflection film prepared by CVD is not limited, and, for example, a SiON film is known. In general, the formation of an intermediate layer by a wet process such as spin coating or screen printing is more convenient and more advantageous in cost than CVD. The upper layer resist for a three-layer process may be positive type or negative type, and the same as a single-layer resist generally used can be used.

The underlayer film according to the present embodiment can also be used as an antireflection film for usual single-layer resists or an underlying material for suppression of pattern collapse. The underlayer film is excellent in etching resistance for an underlying process and can be expected to also function as a hard mask for an underlying process.

In the case of forming a resist film from the above photoresist material, a wet process such as spin coating or screen printing is preferably used, as in the case of forming the above underlayer film. After coating with the resist material by spin coating or the like, prebaking is generally performed. This prebaking is preferably performed at 80 to 180° C. in the range of 10 to 300 seconds. Then, exposure, post-exposure baking (PEB), and development can be performed according to a conventional method to obtain a resist pattern. The thickness of the resist film is not particularly limited, and in general, is preferably 30 to 500 nm and more preferably 50 to 400 nm.

The exposure light can be arbitrarily selected and used according to the photoresist material to be used. General examples thereof can include a high energy ray having a wavelength of 300 nm or less, specifically, excimer laser of 248 nm, 193 nm, or 157 nm, soft x-ray of 3 to 20 nm, electron beam, and X-ray.

In a resist pattern formed by the above method, pattern collapse is suppressed by the underlayer film. Therefore, use of the underlayer film according to the present embodiment can produce a finer pattern and can reduce an exposure amount necessary for obtaining the resist pattern.

Next, etching is performed with the obtained resist pattern as a mask. Gas etching is preferably used as the etching of the underlayer film in a two-layer process. The gas etching is suitably etching using oxygen gas. In addition to oxygen gas, an inert gas such as He or Ar, or CO, CO₂, NH₃, SO₂, N₂, NO₂, or H₂ gas may be added. Alternatively, the gas etching may be performed with only CO, CO₂, NH₃, N₂, NO₂, or H₂ gas without the use of oxygen gas. Particularly, the latter gas is preferably used for side wall protection in order to prevent the undercut of pattern side walls.

On the other hand, gas etching is also preferably used as the etching of the intermediate layer in a three-layer process. The same gas etching as described in the above two-layer process is applicable. Particularly, it is preferable to process the intermediate layer in a three-layer process by using chlorofluorocarbon-based gas and using the resist pattern as a mask. Then, as mentioned above, for example, the underlayer film can be processed by oxygen gas etching with the intermediate layer pattern as a mask.

Here, in the case of forming an inorganic hard mask intermediate layer film as the intermediate layer, a silicon oxide film, a silicon nitride film, or a silicon oxynitride film (SiON film) is formed by CVD, ALD, or the like. A method for forming the nitride film is not particularly limited, and for example, a method described in Japanese Patent Application Laid-Open No. 2002-334869 (Patent Literature 9) or WO 2004/066377 (Patent Literature 10) can be used. Although a photoresist film can be formed directly on such an intermediate layer film, an organic antireflection film (BARC) may be formed on the intermediate layer film by spin coating and a photoresist film may be formed thereon.

A polysilsesquioxane-based intermediate layer is also suitably used as the intermediate layer. By imparting effects as an antireflection film to the resist intermediate layer film, there is a tendency to make it possible to effectively suppress reflection. A specific material for the polysilsesquioxane-based intermediate layer is not limited, and, for example, a material described in Japanese Patent Laid-Open No. 2007-226170 (Patent Literature 11) or Japanese Patent Laid-Open No. 2007-226204 (Patent Literature 12) can be used.

The subsequent etching of the substrate can also be performed by a conventional method. For example, the substrate made of SiO₂ or SiN can be etched mainly using chlorofluorocarbon-based gas, and the substrate made of p-Si, Al, or W can be etched mainly using chlorine- or bromine-based gas. In the case of etching the substrate with chlorofluorocarbon-based gas, the silicon-containing resist of the two-layer resist process or the silicon-containing intermediate layer of the three-layer process is stripped at the same time with substrate processing. On the other hand, in the case of etching the substrate with chlorine- or bromine-based gas, the silicon-containing resist film or the silicon-containing intermediate layer is separately stripped and in general, stripped by dry etching using chlorofluorocarbon-based gas after substrate processing.

A feature of the underlayer film of the present embodiment is that it is excellent in etching resistance of the substrates. The substrate can be arbitrarily selected for use from publicly known ones and is not particularly limited. Examples thereof include Si, α-Si, p-Si, SiO₂, SiN, SiON, W, TiN, and Al. The substrate may be a laminate having a film to be processed (substrate to be processed) on a base material (support). Examples of such a film to be processed include, but not particularly limited to, various low-k films such as Si, SiO₂, SiON, SiN, p-Si, α-Si, W, W—Si, Al, Cu, and Al—Si, and stopper films thereof. A material different from that for the base material (support) is generally used. The thickness of the substrate to be processed or the film to be processed is not particularly limited and is generally preferably about 50 to 1,000,000 nm, and more preferably 75 to 50,000 nm.

The composition of the present embodiment can be prepared by adding each of the above components and mixing them using a stirrer or the like. When the composition of the present embodiment contains a filler or a pigment, it can be prepared by dispersion or mixing using a dispersion apparatus such as a dissolver, a homogenizer, and a three-roll mill.

### [Optical Component]

The composition of the present embodiment is preferably used for optical component formation. That is, an optical component of the present embodiment includes the composition of the present embodiment.

Examples of the above optical component include, but not particularly limited to, a component in the form of a film or a sheet, a plastic lens such as a prism lens, a lenticular lens, a microlens, a Fresnel lens, a viewing angle control lens, and a contrast improving lens, a phase difference film, a film for electromagnetic wave shielding, a prism, an optical fiber, a solder resist for flexible printed wiring, a plating resist, an interlayer insulating film for multilayer printed circuit boards, a photosensitive optical waveguide, a liquid crystal display, an organic electroluminescent (EL) display, an optical semiconductor (LED) element, a solid state image sensing element, an organic thin film solar cell, a dye sensitized solar cell, and an organic thin film transistor (TFT). Compound (1) is suitably used as a material for forming an embedded film and a smoothed film on a photodiode, a smoothed film in front of or behind a color filter, a microlens, and a flattened film and a conformal film on a microlens, all of which are members of a solid state image sensing element, to which high refractive index is demanded.

[Method for Purifying Compound (1) and/or Resin (2)]

The method for purifying compound (1) and/or resin (2) of the present embodiment includes: an extraction step of bringing a solution (hereinafter, also simply referred to as “solution (A)”) containing compound (1) and/or resin (2) of the present embodiment and an organic solvent that does not inadvertently mix with water into contact with an acidic aqueous solution, thereby carrying out extraction. More specifically, in the purification method of the present embodiment, compound (1) and/or resin (2) of the present embodiment is dissolved in an organic solvent that does not inadvertently mix with water, the solution is brought into contact with an acidic aqueous solution, thereby carrying out extraction treatment to transfer the metal fraction included in solution (A) to the aqueous phase, and purification is carried out by separating the organic phase and the aqueous phase. Through the purification method of the present embodiment, the content of various metals in the compound or the resin of the present embodiment can be significantly reduced.

In the present embodiment, the “organic solvent that does not inadvertently mix with water” means that the solubility in water at 20° C. is less than 50% by mass, and preferably less than 25% by mass from the viewpoint of productivity. The organic solvent that does not inadvertently mix with water is not particularly limited, but is preferably an organic solvent that is safely applicable to semiconductor manufacturing processes. The amount of the organic solvent used is usually about 1 to 100 times the amount of compound (1) and/or resin (2) of the present embodiment in terms of mass.

Specific examples of the organic solvent include, but not limited to, those described in International Publication No. WO2015/080240, for example. These solvents are used alone as one kind or in combination of two or more kinds. Among them, toluene, 2-heptanone, cyclohexanone, cyclopentanone, methylisobutylketone, PGMEA, ethyl acetate, and the like are preferable, and cyclohexanone and PGMEA are more preferable.

The acidic aqueous solution to be used is appropriately selected from aqueous solutions in which generally known organic or inorganic compounds are dissolved in water. Examples thereof include those described in International Publication No. WO 2015/080240. These acidic aqueous solutions are used alone as one kind or in combination of two or more kinds. Among them, aqueous solutions of sulfuric acid, nitric acid, and a carboxylic acid such as acetic acid, oxalic acid, tartaric acid, and citric acid are preferable; aqueous solutions of sulfuric acid, oxalic acid, tartaric acid, and citric acid are still more preferable; and an aqueous solution of oxalic acid is further preferable. It is considered that a polyvalent carboxylic acid such as oxalic acid, tartaric acid, and citric acid coordinates with metal ions and provides a chelating effect, and thus is capable of removing more metals. In addition, as the water used herein, water, the metal content of which is small, such as ion exchanged water, is suitably used according to the purpose of the present invention.

The pH of the acidic aqueous solution to be used in the present embodiment is not particularly limited, but the pH range is preferably about 0 to 5 and more preferably about 0 to 3 in general.

The amount of the acidic aqueous solution to be used in the present embodiment is not particularly limited, but when the amount is too small, it is required to increase the number of extraction treatments for removing metals, and on the other hand, when the amount of the aqueous solution is too large, the entire fluid volume becomes large, which may cause operational problems. In general, the amount of the acidic aqueous solution used is preferably 10 to 200% by mass and more preferably 20 to 100% by mass, based on solution (A).

In the purification method of the present embodiment, the metal fraction is extracted by bringing the above acidic aqueous solution into contact with solution (A), for example.

Usually, the temperature at the time of carrying out extraction treatment is preferably 20 to 90° C. and more preferably 30 to 80° C. The extraction operation is carried out, for example, by thoroughly mixing the solutions by stirring or the like and then leaving the obtained mixed solution to stand still. Thereby, the metal fraction contained in solution (A) is transferred to the aqueous phase. Also, by this operation, the acidity of the solution is lowered, and the degradation of compound (1) and/or resin (2) of the present embodiment can be suppressed.

The obtained mixture is separated into an aqueous phase and an organic phase containing compound (1) and/or resin (2) of the present embodiment and the organic solvent, and thus the organic phase containing compound (1) and/or resin (2) of the present embodiment and the organic solvent is recovered by decantation or the like. The time for leaving the solution to stand still is not particularly limited, but is preferably 1 minute or more, more preferably 10 minutes or more, and still more preferably 30 minutes or more, for example. In addition, while the extraction treatment may be carried out only once, it is also effective to repeat mixing, leaving-to-stand-still, and separating operations multiple times.

When such extraction treatment is carried out using the acidic aqueous solution, after the treatment, it is preferable to further subject the recovered organic phase, which has been extracted from the acidic aqueous solution and contains compound (1) and/or resin (2) of the present embodiment and the organic solvent, to extraction treatment with water. The extraction treatment is carried out by thoroughly mixing the organic phase and water by stirring or the like and then leaving the obtained mixed solution to stand still. The obtained solution is separated into an aqueous phase and a solution phase containing compound (1) and/or resin (2) of the present embodiment and the organic solvent, and thus the solution phase containing compound (1) and/or resin (2) of the present embodiment and the organic solvent is recovered by decantation or the like. In addition, as the water used herein, water, the metal content of which is small, such as ion exchanged water, is preferable according to the purpose of the present invention. While the extraction treatment may be carried out only once, it is also effective to repeat mixing, leaving-to-stand-still, and separating operations multiple times. The proportions of both used in the extraction treatment and the temperature, time, and other conditions are not particularly limited, and may be the same as those of the previous contact treatment with the acidic aqueous solution.

Water mixing into the thus-obtained solution containing compound (1) and/or resin (2) of the present embodiment and the organic solvent can be easily removed by performing vacuum distillation or a like operation. Also, if required, the concentration of compound (1) and/or resin (2) of the present embodiment can be regulated to be any concentration by adding an organic solvent.

For the method for obtaining compound (1) and/or resin (2) of the present embodiment alone from the obtained solution containing compound (1) and/or resin (2) of the present embodiment and the organic solvent, a publicly known method can be carried out, such as reduced-pressure removal, separation by reprecipitation, and a combination thereof. A publicly known treatment such as concentration operation, filtration operation, centrifugation operation, and drying operation can be carried out, if required.

### EXAMPLES

The present embodiment will be described in more detail with reference to examples below. However, the present embodiment is not limited to these examples by any means.

### (Carbon Concentration and Oxygen Concentration)

The carbon concentration and the oxygen concentration (% by mass) of the compound or the resin were measured by using an organic elemental analysis device “CHN Coder MT-6” (product name, manufactured by Yaic. Yanaco).

### (LC-MS Analysis: Molecular Weight Measurement)

The molecular weight of the compound or the resin was measured by liquid chromatograph mass spectroscopy (hereinafter, also simply referred to the “LC-MS analysis”) using an analysis device “Acquity UPLC/MALDI-Synapt HDMS” (product name, manufactured by Waters Corporation).

### (Mn, Mw, and Mw/Mn)

The Mn, Mw and Mw/Mn were determined in terms of polystyrene by gel permeation chromatography (GPC) analysis under the following measurement conditions.

Apparatus: “Shodex GPC-101 model” (product name, manufactured by Showa Denko K.K.)

Column: “KF-80M”×3 (product name, manufactured by Showa Denko K.K.)

Eluent: tetrahydrofuran (hereinafter, also referred to as “THF”)

Flow rate: 1 mL/min

Temperature: 40° C.

### (ICP-MS)

Measurement was carried out using an induction coupled plasma mass spectrometer (hereinafter, also referred to as “ICP-MS”) “ELAN DRC II” (product name, manufactured by Perkin Elmer Inc.).

### (Evaluation of Solubility)

At 23° C., the compound or the resin was dissolved in propylene glycol monomethyl ether (hereinafter also referred to as “PGME”) to form a 5 massc solution. Subsequently, the solubility after leaving the solution to stand still at 5° C. for 30 days was evaluated according to the following criteria.

Evaluation A: no precipitate was visually confirmed

Evaluation C: precipitates were visually confirmed

### [Synthesis of Compound (1)]

### (Example A1) Synthesis of Compound (BiP-1)

To a container (internal capacity: 1000 mL) equipped with a stirrer, a condenser tube, and a burette, 154 g of 3,3′-dimethylbiphenyl-4,4′-diol (a reagent manufactured by Sigma-Aldrich Co. LLC.), 12 g of sulfuric acid, 11 g of benzaldehyde (a reagent manufactured by Sigma-Aldrich Co. LLC.), and 600 g of 1-methoxy-2-propanol were added, and the contents were stirred at 100° C. for 6 hours and reacted to obtain a reaction liquid. The reaction liquid was cooled, 1600 g of ethyl acetate was added thereto, followed by concentration and separation by column chromatography to obtain 25 g of objective compound (BiP-1) represented by the following formula (BiP-1).

The molecular weight of the obtained compound (BiP-1) measured by the method according to the above “LC-MS analysis” was 516. In addition, the carbon concentration of the obtained compound (BiP-1) was 81.4% by mass, and the oxygen concentration thereof was 12.4% by mass.

The peaks shown in Table 1 were found by ¹H-NMR (500 MHz, DMSO-d₆) measurement performed on the obtained compound (BiP-1), and the compound (BiP-1) was confirmed to have a chemical structure of the following formula (BiP-1).

Evaluation of solubility was conducted on the above compound (BiP-1). The results are shown in Table 1.

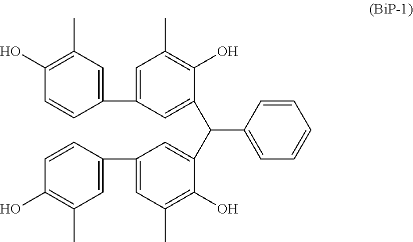 [SMILES: Cc1cc(-c2cc(C)c(O)c(C(c3ccccc3)c3cc(-c4ccc(O)c(C)c4)cc(C)c3O)c2)ccc1O]

### (Examples A2 to A7) Synthesis of Compounds (BiP-2), (BiP-3), (BiP-4), (BiP-5), (BiP-6), and (BiP-7)

Objective compounds (BiP-2), (BiP-3), (BiP-4), (BiP-5), (BiP-6), and (BiP-7) were obtained in the same manner except that benzaldehyde was changed to the raw materials shown in Table 1 below. The molecular weights, carbon concentrations, oxygen concentrations, and ¹H-NMR (500 MHz, DMSO-d₆) measurement results of the obtained compounds are shown in Table 1 below. The objective compounds were confirmed to have chemical structures of the following formulae (BiP-2), (BiP-3), (BiP-4), (BiP-5), (BiP-6), and (BiP-7), respectively.

Evaluation of solubility was conducted on the above compounds (BiP-2), (BiP-3), (BiP-4), (BiP-5), (BiP-6), and (BiP-7). The results are shown in Table 1.

TABLE 1

| | | | | Elemental analysis | | | |
|---|---|---|---|---|---|---|---|
| | | | LC-MS | (mass %) | | | |
| | Com- | | Molecular | Carbon | Oxygen | | |
| | pound | Aldehyde species | Weight | concentration | concentration | ¹H-NMR δ(ppm) | Solubility |
| Example | BiP-1 | Benzaldehyde | 516 | 81.4 | 12.4 | 2.2-2.5(12H, —CH₃), 6.4(1H, CH), 6.7- | A |
| A1 | | | | | | 7.6(15H, Ph—H), 8.3-9.2(4H, —OH) | |
| Example | BiP-2 | ρ-Tolualdehyde | 530 | 81.5 | 12.1 | 2.1-2.5(15H, —CH₃), 6.4(1H, CH), 6.7- | A |
| A2 | | | | | | 7.6(14H, Ph—H), 8.3-9.2(4H, —OH) | |
| Example | BiP-3 | ρ-(n-propyl)- | 558 | 81.7 | 11.5 | 0.9(3H, —CH₂—CH₂—CH₃), 1.6(2H, | A |
| A3 | | Benzaldehyde | | | | —CH₂—CH₂—CH₃), 2.2-2.5(12H, | |
| | | | | | | —CH₃), 2.6(2H, —CH₂—CH₂—CH₃), | |
| | | | | | | 6.4(1H, CH), 6.7-7.6(14H, Ph—H), | |
| | | | | | | 8.3-9.2(4H, —OH) | |
| Example | BiP-4 | ρ-(n-butyl)- | 572 | 81.8 | 11.2 | 0.9(3H, —CH₂—CH₂—CH₂—CH₃), | A |
| A4 | | Benzaldehyde | | | | 1.3(2H, —CH₂—CH₂—CH₂—CH₃), | |
| | | | | | | 1.6(2H, —CH₂—CH₂—CH₂—CH₃), | |
| | | | | | | 2.2-2.5(12H, —CH₃), 2.6(2H, | |
| | | | | | | —CH₂—CH₂—CH₂—CH₃), 6.4(1H, | |
| | | | | | | CH), 6.7~7.6(14Hs,Ph—H), 8.3- | |
| | | | | | | 9.2(4H,—OH) | |
| Example | BiP-5 | 4-Biphenyl- | 592 | 83.1 | 10.8 | 2.2-2.5(12H, —CH₃), 6.4(1H, CH), | A |
| A5 | | aldehyde | | | | 6.7-7.6(19H, Ph—H), 8.3-9.2(4H, | |
| | | | | | | —OH) | |
| Example | BiP-6 | 2-Naphth- | 566 | 82.7 | 11.3 | 2.2-2.5 (12H,—CH₃), 6.4(1H,CH), | A |
| A6 | | aldehyde | | | | 6.7-7.6(17H,Ph—H),8.3~9.2(4H, | |
| | | | | | | —OH) | |
| Example | BiP-7 | 4-Hydroxybenz- | 532 | 78.9 | 15.0 | 2.2-2.5 (12H,—CH₃), 6.4(1H,CH), | A |
| A7 | | aldehyde | | | | 6.9-7.6(14H, Ph—H), 8.3-9.2(5H, | |
| | | | | | | —OH) | |

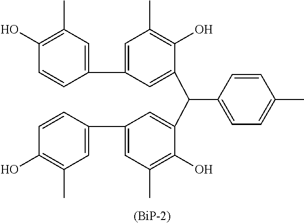

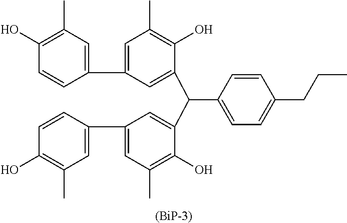

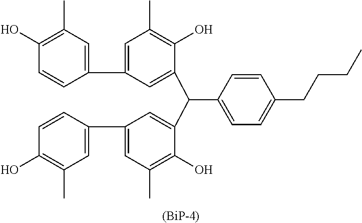

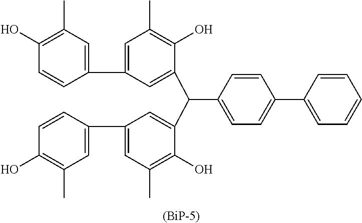

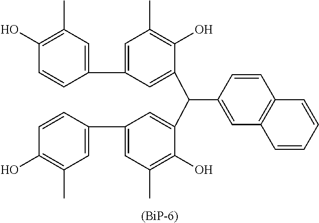

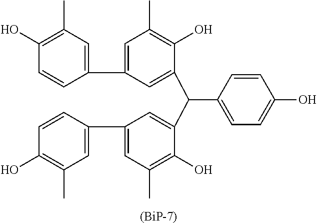

### (Examples A8 to A10) Synthesis of Compounds (BiP-8), (BiP-9), and (BiP-10)

Objective compounds (BiP-8), (BiP-9), and (BiP-10) were obtained in the same manner as in Example A5 except that 3,3′-dimethylbiphenyl-4,4′-diol was changed to the raw materials shown in phenol species of Table 2 below. The molecular weights, carbon concentrations, oxygen concentrations, and ¹H-NMR (500 MHz, DMSO-d₅) measurement results of the obtained compounds are shown in Table 2 below. The objective compounds were confirmed to have chemical structures of the following formulae (BiP-8), (BiP-9), and (BiP-10), respectively. Evaluation of solubility was conducted on the above compounds (BiP-8), (BiP-9), and (BiP-10). The results are shown in Table 2.

TABLE 2

| | | | | Elemental analysis | | | |
|---|---|---|---|---|---|---|---|
| | | | LC-MS | (mass %) | | | |
| | Com- | | Molecular | Carbon | Oxygen | | |
| | pound | Phenol species | Weight | concentration | concentration | ¹H-NMR | Solubility |
| Example | BiP-8 | 2,2-Bis(4-hydroxy-3- | 676 | 83.4 | 9.5 | 1.7(12H, —CH₃, 2.1-2.2(12H, | A |
| A8 | | methylphenyl)propane | | | | Ph—CH₃), 6.4(1H, —CH), 6.7- | |
| | | | | | | 7.8(19H, Ph—H), 8.3-9.2(4H, | |
| | | | | | | —OH) | |
| Example | BiP-9 | 4.4′-Methylenebis- | 676 | 83.4 | 9.5 | 2.1-2.3(24H, Ph—CH₃), 4.0(4H, | A |
| A9 | | (2,5-dimethylphenol) | | | | —CH₂—), 6.4(1H, —CH), 6.7- | |
| | | | | | | 7.8(15H, Ph—H), 8.3-9.2(4H, | |
| | | | | | | —OH) | |
| Example | BiP-10 | 3,3′-Diphenylbi- | 840 | 87.1 | 7.6 | 6.5(1H, —CH), 7.0-7.8(39H, | A |
| A10 | | phenyl-4,4′-diol | | | | Ph—H), 8.3-9.2(4H, —OH) | |

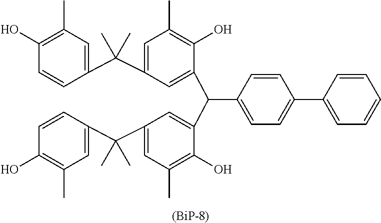

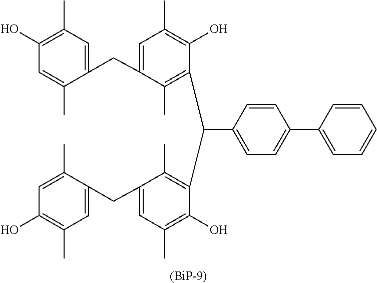

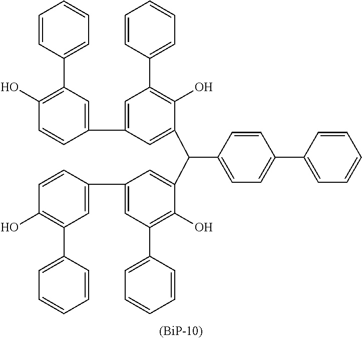

### (Example A11) Synthesis of Compound (BiP-1-MeBOC)

To a container (internal capacity: 500 mL) equipped with a stirrer, a condenser tube, and a burette, 8.0 g (15.5 mmol) of compound (BiP-1) obtained by the method described in Example A1 and 13.5 g (68 mmol) of tert-butyl bromoacetate (manufactured by Sigma-Aldrich Co. LLC.) were added with 200 mL of acetone, 9.5 g (68 mmol) of potassium carbonate (manufactured by Sigma-Aldrich Co. LLC.) and 1.0 g of 18-crown-6 were added thereto, and the contents were reacted by being stirred under reflux for 3 hours to obtain a reaction liquid. Next, the reaction liquid was concentrated, and the reaction product was precipitated by the addition of 200 g of pure water to the concentrate, cooled to room temperature, and then filtered to separate solid matter.

The obtained solid matter was dried, and then separated and purified by column chromatography to obtain 1.2 g of the following formula (BiP-MeBOC).

The following peaks were found by ¹H-NMR (500 MHz, DMSO-d₆) measurement performed on the obtained compound (BiP-MeBOC), and the compound was confirmed to have a chemical structure of the following formula (BiP-MeBOC).

δ (ppm) 1.4 (36H, O—C—CH₃), 2.2-2.5 (12H, Ph-CH₃), 5.0 (8H, O—CH₂—C), 6.4 (1H, C—H), 6.7-7.6 (15H, Ph-H)

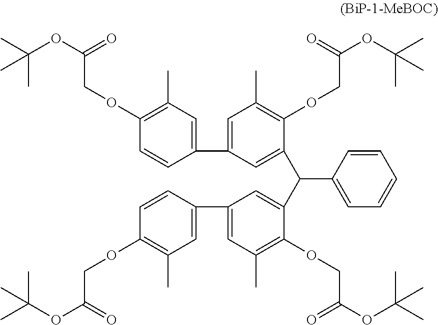 [SMILES: Cc1cc(-c2cc(C)c(OCC(=O)OC(C)(C)C)c(C(c3ccccc3)c3cc(-c4ccc(OCC(=O)OC(C)(C)C)c(C)c4)cc(C)c3OCC(=O)OC(C)(C)C)c2)ccc1OCC(=O)OC(C)(C)C]

### (Example A12) Synthesis of Compound (BiP-1-BOC)

To a container (internal capacity: 300 mL) equipped with a stirrer, a condenser tube, and a burette, 8.0 g (15.5 mmol) of compound (BiP-1) obtained by the method described in Example A1 and 13.7 g (62.8 mmol) of di-tert-butyl dicarbonate (manufactured by Sigma-Aldrich Co. LLC.) were added with 100 mL of acetone, 8.64 g (62.5 mmol) of potassium carbonate (manufactured by Sigma-Aldrich Co. LLC.) was added thereto, and the contents were stirred at 20° C. for 6 hours and reacted to obtain a reaction liquid. Next, the reaction liquid was concentrated, and the reaction product was precipitated by the addition of 100 g of pure water to the concentrate, cooled to room temperature, and then filtered to separate solid matter.

The obtained solid matter was filtered, dried, and then separated and purified by column chromatography, thereby obtaining 1.0 g of the objective compound (BiP-1-BOC) represented by the following formula (BiP-1-BOC).

The following peaks were found by ¹H-NMR (500 MHz, DMSO-d₆) measurement performed on the obtained compound (BiP-1-BOC), and the compound was confirmed to have a chemical structure of the following formula (BiP-1-BOC).

δ (ppm) 1.4 (36H, O—C—CH₃), 2.2-2.5 (12H, Ph-CH₃), 6.4 (1H, C—H), 6.7-7.6 (15H, Ph-H)

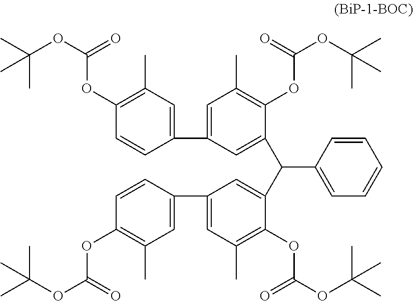 [SMILES: Cc1cc(-c2cc(C)c(OC(=O)OC(C)(C)C)c(C(c3ccccc3)c3cc(-c4ccc(OC(=O)OC(C)(C)C)c(C)c4)cc(C)c3OC(=O)OC(C)(C)C)c2)ccc1OC(=O)OC(C)(C)C]

### (Example A13) Synthesis of Compound (BiP-1-AL)

To a container (internal capacity: 1000 mL) equipped with a stirrer, a condenser tube, and a burette, 8.0 g (15.5 mmol) of compound (BiP-1) obtained by the method described in Example A1 and 108 g (810 mmol) of potassium carbonate were added with 200 mL of dimethylformamide, 200 g (1.65 mol) of allyl bromide was added thereto, and the reaction liquid was stirred at 110° C. for 24 hours and reacted. Next, the reaction liquid was concentrated, and the reaction product was precipitated by the addition of 500 g of pure water, cooled to room temperature, and then separated by filtration. The obtained solid matter was filtered, dried, and then separated and purified by column chromatography, thereby obtaining 5.1 g of the objective compound (BiP-1-AL) represented by the following formula (BiP-1-AL).

The following peaks were found by ¹H-NMR (500 MHz, DMSO-d₆, internal standard: TMS) measurement performed on the obtained compound, and the compound was confirmed to have a chemical structure of the following formula (BiP-1-AL).

δ (ppm) 2.2-2.5 (12H, Ph-CH₂), 4.7 (8H, —CH₂—), 5.3-5.4 (8H, —C═CH₂), 6.1 (4H, —CH═C), 6.4 (1H, C—H), 6.7-7.6 (15H, Ph-H)

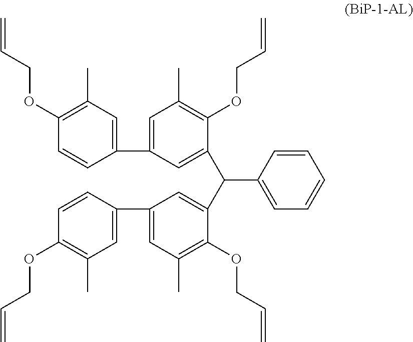 [SMILES: C=CCOc1ccc(-c2cc(C)c(OCC=C)c(C(c3ccccc3)c3cc(-c4ccc(OCC=C)c(C)c4)cc(C)c3OCC=C)c2)cc1C]

### (Example A14) Synthesis of Compound (BiP-1-Ac)

The objective compound (BiP-1-Ac) (5.0 g) represented by the following formula (BiP-1-Ac) was obtained in the same manner as in Example A13 except that 119 g (1.65 mol) of acrylic acid was used instead of 200 g (1.65 mol) of allyl bromide described above.

The following peaks were found by ¹H-NMR (500 MHz, DMSO-d₆, internal standard: TMS) measurement performed on the obtained compound under the above measurement conditions, and the compound was confirmed to have a chemical structure of the following formula (BiP-1-Ac).

δ (ppm) 2.2-2.5 (12H, Ph-CH₃), 5.7 (4H, C═C—H), 6.1-6.2 (8H, —CH═C, C═C—H), 6.4 (1H, C—H), 6.7-7.6 (15H, Ph-H)

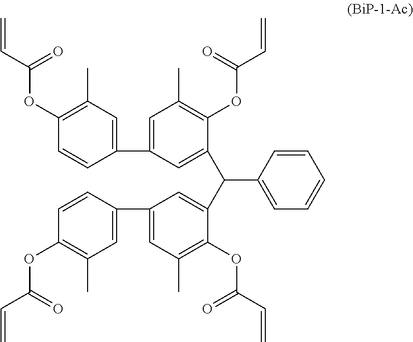 [SMILES: C=CC(=O)Oc1ccc(-c2cc(C)c(OC(=O)C=C)c(C(c3ccccc3)c3cc(-c4ccc(OC(=O)C=C)c(C)c4)cc(C)c3OC(=O)C=C)c2)cc1C]

### (Example A15) Synthesis of Compound (BiP-1-Ea)

To a container (internal capacity: 100 mL) equipped with a stirrer, a condenser tube, and a burette, 6.5 g (12.6 mmol) of compound (BiP-1) obtained by the method described in Example A1, 9.2 g of glycidyl methacrylate, 0.75 g of triethylamine, and 0.08 g of p-methoxyphenol were added with 70 mL of methyl isobutyl ketone, and the contents were warmed to 80° C. and reacted with stirring for 24 hours.

The resultant was cooled to 50° C., and the reaction liquid was added dropwise into pure water. The precipitated solid matter was filtered, dried, and then separated and purified by column chromatography to obtain 1.7 g of the objective compound (BiP-1-Ea) represented by the following formula (BiP-1-Ea).

The obtained compound was confirmed to have a chemical structure of the following formula (BiP-1-Ea) by ¹H-NMR (500 MHz, DMSO-d₆, internal standard: TMS) measurement.

δ (ppm) 2.0 (12H, —CH₃), 2.2-2.5 (12H, —CH₃), 4.0-4.4 (16H, —CH₂—) 4.7 (4H, C—H), 5.8 (4H, —OH), 6.4-6.5 (9H, C—H, C═CH₂), 6.7-7.6 (15H, Ph-H)

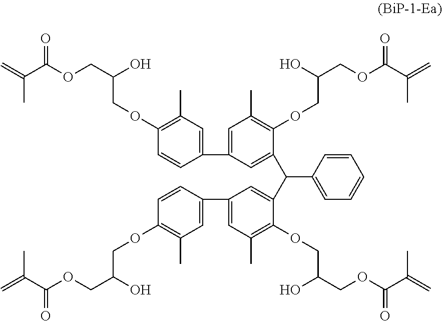 [SMILES: C=C(C)C(=O)OCC(O)COc1ccc(-c2cc(C)c(OCC(O)COC(=O)C(=C)C)c(C(c3ccccc3)c3cc(-c4ccc(OCC(O)COC(=O)C(=C)C)c(C)c4)cc(C)c3OCC(O)COC(=O)C(=C)C)c2)cc1C]

### (Example A16) Synthesis of Compound (BiP-1-Ua)

To a container (internal capacity: 100 mL) equipped with a stirrer, a condenser tube, and a burette, 6.5 g (12.6 mmol) of compound (BiP-1) obtained by the method described in Example A1, 9.2 g of 2-isocyanatoethyl methacrylate, 0.75 g of triethylamine, and 0.08 g of p-methoxyphenol were added with 70 mL of methyl isobutyl ketone, and the contents were warmed to 80° C. and reacted with stirring for 24 hours. The resultant was cooled to 50° C., and the reaction liquid was added dropwise into pure water. The precipitated solid matter was filtered, dried, and then separated and purified by column chromatography to obtain 1.8 g of the objective compound (BiP-1-Ua) represented by the following formula (BiP-1-Ua). The obtained compound was confirmed to have a chemical structure of the following formula (BiP-1-Ua) by ¹H-NMR (500 MHz, DMSO-d₆, internal standard: TMS) measurement.

δ (ppm) 2.0 (12H, —CH₃), 2.2-2.5 (12H, —CH₃), 3.2 (8H, —CH₂—), 4.6 (8H, —CH₂—), 6.4-6.5 (9H, C—H, =CHz), 6.7-7.6 (19H, Ph-H, —NH—)

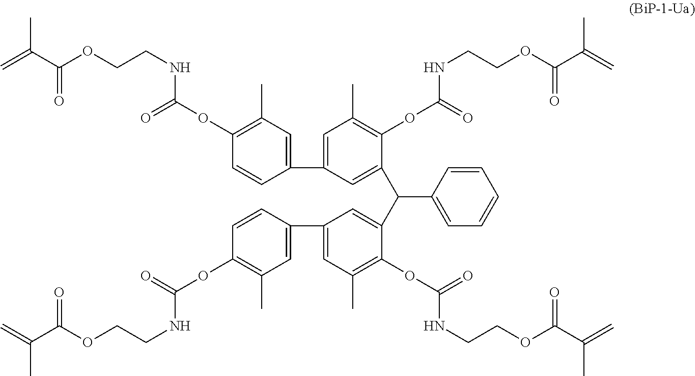 [SMILES: C=C(C)C(=O)OCCNC(=O)Oc1ccc(-c2cc(C)c(OC(=O)NCCOC(=O)C(=C)C)c(C(c3ccccc3)c3cc(-c4ccc(OC(=O)NCCOC(=O)C(=C)C)c(C)c4)cc(C)c3OC(=O)NCCOC(=O)C(=C)C)c2)cc1C]

### (Example A17) Synthesis of Compound (BiP-1-E)

To a container (internal capacity: 100 mL) equipped with a stirrer, a condenser tube, and a burette, 6.5 g (12.6 mmol) of compound (BiP-1) obtained by the method described in Example A1 and 18.0 g (130 mmol) of potassium carbonate were added with 60 mL of dimethylformamide, 8.0 g (65 mol) of 2-chloroethyl acetate was added thereto, and the reaction liquid was stirred at 90° C. for 12 hours and reacted. Next, the reaction liquid was cooled in an ice bath to precipitate crystals, which were then separated by filtration. Subsequently, to a container (internal capacity: 100 mL) equipped with a stirrer, a condenser tube, and a burette, 40 g of the crystals described above, 40 g of methanol, 100 g of THF, and a 24 mass % aqueous sodium hydroxide solution were added. The reaction liquid was stirred for 4 hours under reflux and reacted. Then, the reaction liquid was cooled in an ice bath and concentrated. The precipitated solid matter was filtered, dried, and then separated and purified by column chromatography to obtain 3.8 g of the objective compound (BiP-1-E) represented by the following formula (BiP-1-E). The obtained compound was confirmed to have a chemical structure of the following formula (BiP-1-E) by ¹H-NMR (500 MHz, DMSO-d_{F}, internal standard: TMS) measurement.

δ (ppm) 2.2-2.5 (12H, —CH3), 3.7 (8H, —CH2-), 4.3 (8H, —CH2-), 4.9 (4H, —OH), 6.4 (1H, C—H), 6.7-7.6 (15H, Ph-H)

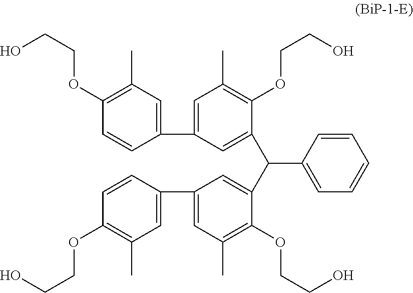 [SMILES: Cc1cc(-c2cc(C)c(OCCO)c(C(c3ccccc3)c3cc(-c4ccc(OCCO)c(C)c4)cc(C)c3OCCO)c2)ccc1OCCO]

### (Example A18) Synthesis of Compound (BiP-1-PX)

To a container (internal capacity: 1000 mL) equipped with a stirrer, a condenser tube, and a burette, 27 g (46 mmol) of the compound (BiP-1) obtained by the method described in Example A1, 78.6 g of iodoanisole, 145.9 g of cesium carbonate, 2.35 g of dimethylglycine hydrochloride, and 0.85 g of copper iodide were added with 400 mL of 1,4-dioxane, and the contents were warmed to 95° C., stirred for 22 hours, and reacted. Next, insoluble matter was filtered off, and the filtrate was concentrated and added dropwise into pure water. The precipitated solid matter was filtered, dried, and then separated and purified by column chromatography to obtain 16 g of compound (BiP-1-M) represented by the following formula (BiP-1-M). The compound (BiP-1-M) was used as an intermediate in the next step.

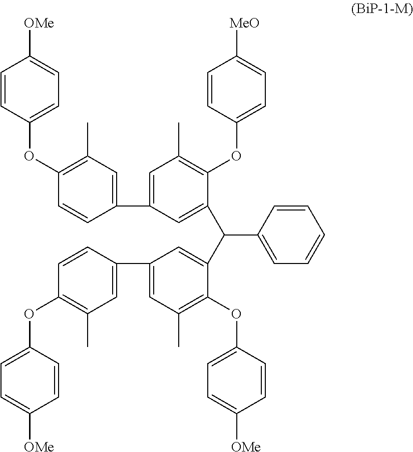 [SMILES: COc1ccc(Oc2ccc(-c3cc(C)c(Oc4ccc(OC)cc4)c(C(c4ccccc4)c4cc(-c5ccc(Oc6ccc(OC)cc6)c(C)c5)cc(C)c4Oc4ccc(OC)cc4)c3)cc2C)cc1]

Next, to a container (internal capacity: 1000 mL) equipped with a stirrer, a condenser tube, and a burette, 16 g of the above-described compound (BiP-1-M) and 80 g of pyridine hydrochloride were added, and the contents were stirred at 190° C. for 2 hours and reacted. Next, 160 mL of hot water was added thereto, and the mixture was stirred to precipitate solid matter. Then, 250 mL of ethyl acetate and 100 mL of water were added thereto, and the mixture was stirred and left to stand still. The separated organic phase was concentrated, dried, and then separated and purified by column chromatography to obtain 12.5 g of the objective compound (BiP-1-PX) represented by the following formula (BiP-1-PX).

The obtained compound was confirmed to have a chemical structure of the following formula (BiP-1-PX) by ¹H-NMR (500 MHz, DMSO-d₆, internal standard: TMS) measurement.

δ (ppm) 2.2-2.5 (12H, —CH₃), 6.4 (1H, C—H), 6.7-7.6 (31H, Ph-H), 9.5 (4H, O—H)

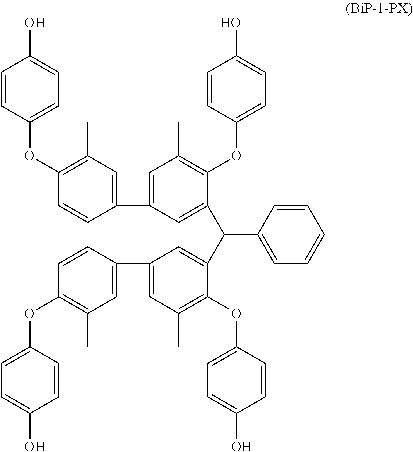 [SMILES: Cc1cc(-c2cc(C)c(Oc3ccc(O)cc3)c(C(c3ccccc3)c3cc(-c4ccc(Oc5ccc(O)cc5)c(C)c4)cc(C)c3Oc3ccc(O)cc3)c2)ccc1Oc1ccc(O)cc1]

### (Example A19) Synthesis of Compound (BiP-1-PE)

The same reaction as in Example A18 was performed except that the above-described compound (BiP-1-E) was used instead of the above-described compound (BiP-1) to obtain 4 g of the objective compound (BiP-1-PE) represented by the following formula (BiP-1-PE).

The obtained compound was confirmed to have a chemical structure of the following formula (BiP-1-PE) by ¹H-NMR (500 MHz, DMSO-d₆, internal standard: TMS) measurement.

δ (ppm) 2.2-2.5 (12H, —CH₃), 3.1 (8H, —CH₂—), 4.3 (8H, —CH₂—), 6.4 (1H, C—H), 6.7-7.6 (31H, Ph-H), 9.5 (4H, O—H)

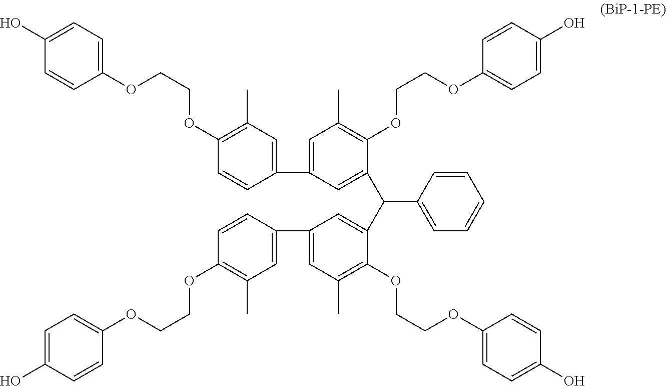 [SMILES: Cc1cc(-c2cc(C)c(OCCOc3ccc(O)cc3)c(C(c3ccccc3)c3cc(-c4ccc(OCCOc5ccc(O)cc5)c(C)c4)cc(C)c3OCCOc3ccc(O)cc3)c2)ccc1OCCOc1ccc(O)cc1]

### (Example A20) Synthesis of Compound (BiP-1-G)

To a container (internal capacity: 100 mL) equipped with a stirrer, a condenser tube, and a burette, 5.4 g (10.5 mmol) of compound (BiP-1) obtained by the method described in Example A1 and 6.2 g (45 mmol) of potassium carbonate were added with 100 mL of dimethylformamide, 4.1 g (45 mmol) of epichlorohydrin was further added thereto, and the obtained reaction liquid was stirred at 90° C. for 6.5 hours and reacted. Next, the solid content was removed from the reaction liquid by filtration, the reaction liquid was cooled in an ice bath to precipitate crystals, which were then filtered, dried, and then separated and purified by column chromatography to obtain 1.9 g of the objective compound (BiP-1-G) represented by the following formula (BiP-1-G).

The following peaks were found by ¹H-NMR (500 MHz, DMSO-d₆, internal standard: TMS) measurement performed on the obtained compound (BiP-1-G), and the compound was confirmed to have a chemical structure of the following formula (BiP-1-G).

δ (ppm) 2.2-3.1 (24H, —CH₃, —CH(CH₂)O), 3.9-4.2 (8H, —CH₂—), 6.4 (1H, C—H), 6.7-7.6 (15H, Ph-H)

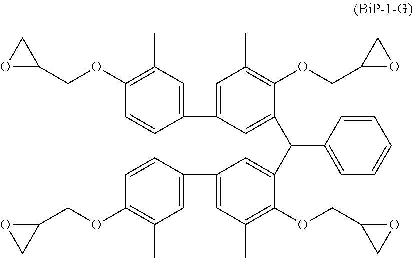 [SMILES: Cc1cc(-c2cc(C)c(OCC3CO3)c(C(c3ccccc3)c3cc(-c4ccc(OCC5CO5)c(C)c4)cc(C)c3OCC3CO3)c2)ccc1OCC1CO1]

### (Example A21) Synthesis of Compound (BiP-1-GE)

The same reaction as in Example A20 was performed except that compound (BiP-1-E) was used instead of compound (BiP-1) to obtain 1.5 g of the objective compound (BiP-1-GE) represented by the following formula (BiP-1-GE).

The obtained compound was confirmed to have a chemical structure of the following formula (BiP-1-GE) by ¹H-NMR (500 MHz, DMSO-d₆, internal standard: TMS) measurement.

δ (ppm) 2.2-2.8 (24H, —CH₃, —CH(CH₂)O), 3.3-4.3 (24H, —CH₂—), 6.4 (1H, C—H), 6.7-7.6 (15H, Ph-H)

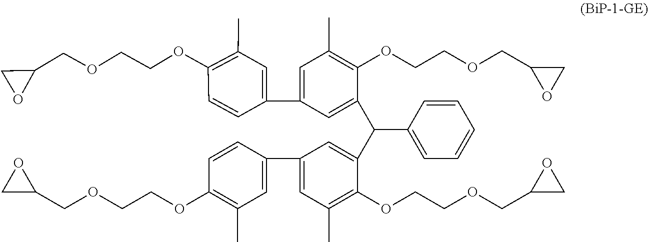 [SMILES: Cc1cc(-c2cc(C)c(OCCOCC3CO3)c(C(c3ccccc3)c3cc(-c4ccc(OCCOCC5CO5)c(C)c4)cc(C)c3OCCOCC3CO3)c2)ccc1OCCOCC1CO1]

### (Example A22) Synthesis of Compound (BiP-1-SX)

To a container (internal capacity: 100 mL) equipped with a stirrer, a condenser tube, and a burette, 5.4 g (10.5 mmol) of compound (BiP-1) obtained by the method described in Example A1 and 6.4 g of vinylbenzyl chloride “CMS-P” (product name, manufactured by AGC Seimi Chemical Co., Ltd.) were added with 50 mL of dimethylformamide, the contents were warmed to 50° C., 8.0 g of 28 mass % sodium methoxide (methanol solution) was added thereto with a dropping funnel over 20 minutes while being stirred, and the reaction liquid was stirred at 50° C. and reacted for 1 hour. Next, 1.6 g of 28 mass % sodium methoxide (methanol solution) was added thereto, the reaction liquid was warmed to 60° C. and stirred for 3 hours, 1.2 g of 85 mass % phosphoric acid was further added thereto followed by stirring for 10 minutes and cooling to 40° C., and the reaction liquid was added dropwise into pure water. The precipitated solid matter was filtered, dried, and then separated and purified by column chromatography to obtain 1.8 g of the objective compound (BiP-1-SX) represented by the following formula (BiP-1-SX).

The obtained compound was confirmed to have a chemical structure of the following formula (BiP-1-SX) by ¹H-NMR (500 MHz, DMSO-d₆, internal standard: TMS) measurement.

δ (ppm) 2.2-2.5 (12H, —CH₃), 5.1-5.8 (16H, —CH₂—, —C═CH₂), 6.4 (1H, C—H), 6.7-7.9 (35H, Ph-H, —CH═C)

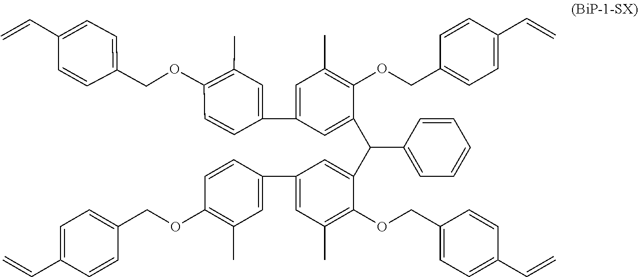 [SMILES: C=Cc1ccc(COc2ccc(-c3cc(C)c(OCc4ccc(C=C)cc4)c(C(c4ccccc4)c4cc(-c5ccc(OCc6ccc(C=C)cc6)c(C)c5)cc(C)c4OCc4ccc(C=C)cc4)c3)cc2C)cc1]

### (Example A23) Synthesis of Compound (BiP-1-SE)

The same reaction as in Example A22 was performed except that the above compound (BiP-1-E) was used instead of the above compound (BiP-1) to obtain 1.5 g of the objective compound (BiP-1-SE) represented by the following formula (BiP-1-SE).

The obtained compound was confirmed to have a chemical structure of the following formula (BiP-1-SE) by ¹H-NMR (500 MHz, DMSO-d₆, internal standard: TMS) measurement.

δ (ppm) 2.2-2.5 (12H, —CH₃), 3.8 (8H, —CH₂—), 4.3 (8H, —CH₂—), 4.8 (8H, —CH₂—), 5.3 (4H, —C═CH), 5.8 (4H, —C═CH), 6.4 (1H, C—H), 6.7-7.6 (35H, Ph-H, —CH═C)

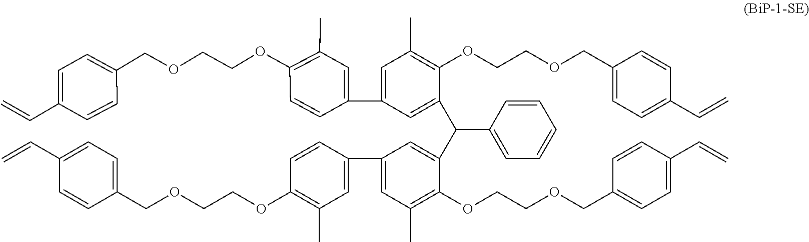 [SMILES: C=Cc1ccc(COCCOc2ccc(-c3cc(C)c(OCCOCc4ccc(C=C)cc4)c(C(c4ccccc4)c4cc(-c5ccc(OCCOCc6ccc(C=C)cc6)c(C)c5)cc(C)c4OCCOCc4ccc(C=C)cc4)c3)cc2C)cc1]

### (Example A24) Synthesis of Compound (BiP-1-Pr)

To a container (internal capacity: 300 mL) equipped with a stirrer, a condenser tube, and a burette, 5.4 g (10.5 mmol) of compound (BiP-1) obtained by the method described in Example A1 and 7.9 g (66 mmol) of propargyl bromide were added with 100 mL of dimethylformamide, and the contents were stirred at room temperature for 3 hours and reacted to obtain a reaction liquid. Next, the reaction liquid was concentrated, and the reaction product was precipitated by the addition of 300 g of pure water to the concentrate, cooled to room temperature, and then filtered to separate solid matter.

The obtained solid matter was filtered, dried, and then separated and purified by column chromatography, thereby obtaining 2.8 g of the objective compound (BiP-1-Pr) represented by the following formula (BiP-1-Pr).

The following peaks were found by ¹H-NMR (500 MHz, DMSO-d₆, internal standard: TMS) measurement performed on the obtained compound (BiP-1-Pr), and the compound was confirmed to have a chemical structure of the following formula (BiP-1-Pr).

δ (ppm) 2.2-2.5 (12H, —CH₃), 3.4 (4H, C═CH), 4.7 (8H, —CH₂—), 6.4 (1H, C—H), 6.7-7.6 (15H, Ph-H)

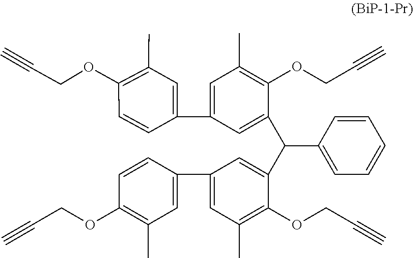 [SMILES: C#CCOc1ccc(-c2cc(C)c(OCC#C)c(C(c3ccccc3)c3cc(-c4ccc(OCC#C)c(C)c4)cc(C)c3OCC#C)c2)cc1C]

### [Synthesis of Resin (2)]

### (Example B1) Synthesis of Resin (RBiP-1)

To a container (internal capacity: 1000 mL) equipped with a stirrer, a condenser tube, and a burette, 154 g of 3,3′-dimethylbiphenyl-4,4′-diol (a reagent manufactured by Sigma-Aldrich Co. LLC.), 12 g of sulfuric acid, 11 g of benzaldehyde (a reagent manufactured by Sigma-Aldrich Co. LLC.), and 600 g of 1-methoxy-2-propanol were added, and the contents were stirred at 100° C. for 6 hours and reacted to obtain a reaction liquid. The reaction liquid was cooled, 1600 g of ethyl acetate was added thereto, followed by concentration, and 1000 g of heptane was added thereto to precipitate solid matter, which was then separated to obtain 96.0 g of the objective resin (RBiP-1) represented by the following formula (RBiP-1).

As a result of measuring Mw and Mw/Mn of the obtained resin (RBiP-1) by the above method, Mw=1290, and Mw/Mn=1.29.

Solubility test was conducted for the above resin (RBiP-1). The results are shown in Table 3.

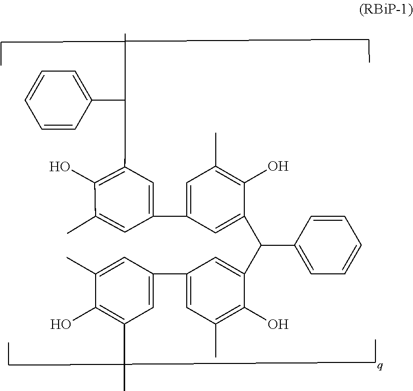

(In formula (RBiP-1), q represents the number of repeat units.)

### (Examples B2 to B7) Synthesis of Resins (RBiP-2), (RBiP-3), (RBiP-4), (RBiP-5), (RBiP-6), and (RBiP-7)

Objective resins (RBiP-2), (RBiP-3), (RBiP-4), (RBiP-5), (RBiP-6), and (RBiP-7) were obtained in the same manner as in Example B1 except that benzaldehyde was changed to the raw materials shown in Table 3 below. The molecular weights and ¹H-NMR (500 MHz, DMSO-d₆) measurement results of the obtained resins are shown in Table 3 below. The objective resins were confirmed to have chemical structures of the following formulae (RBiP-2), (RBiP-3), (RBiP-4), (RBiP-5), (RBiP-6), and (RBiP-7), respectively. Solubility test was conducted for the above resins (RBiP-2) to (RBiP-7). The results are shown in Table 3.

### (Example B8) Synthesis of Resin (RBiP-8)

A four necked flask (internal capacity: 1 L) equipped with a Dimroth condenser tube, a thermometer, and a stirring blade and having a detachable bottom was prepared. To this four necked flask, 25.8 g (50 mmol) of compound (BisP-1) obtained by the method described in Example A1, 21.0 g (280 mmol as formaldehyde) of a 40 mass % aqueous formaldehyde solution (manufactured by Mitsubishi Gas Chemical Company, Inc.), and 0.97 mL of 98 mass % sulfuric acid (manufactured by Kanto Chemical Co., Inc.) were added in a nitrogen stream, and the mixture was allowed to react for 7 hours while being refluxed at 100° C. at normal pressure. Subsequently, 180.0 g of orthoxylene (special grade reagent manufactured by Wako Pure Chemical Industries, Ltd.) was added as a diluting solvent to the reaction liquid, and the mixture was left to stand still, followed by removal of an aqueous phase as a lower phase. Neutralization and washing with water were further performed, and orthoxylene was distilled off under reduced pressure to obtain 20.5 g of a brown solid resin (RBiP-8). The molecular weight and ¹H-NMR (500 MHz, DMSO-d₆) measurement result of the obtained resin are shown in Table 3 below.

TABLE 3

| | | | GPC | | | |
|---|---|---|---|---|---|---|
| | Resin | Aldehyde species | Mw | Mw/Mn | ¹H-NMR δ(ppm) | Solubility |
| Example | RBiP-1 | Benzaldehyde | 1290 | 1.29 | 2.2-2.5(—CH₃), 6.4(CH), 6.7- | A |
| B1 | | | | | 7.6(Ph—H), 8.3-9.2(—OH) | |
| Example | RBiP-2 | ρ-Tolualdehyde | 1159 | 1.18 | 2.1-2.5(—CH₃), 6.4(CH), 6.7- | A |
| B2 | | | | | 7.6(Ph—H), 8.3-9.2(—OH) | |
| Example | RBiP-3 | ρ-(n-propyl) | 989 | 1.10 | 0.9(—CH₂—CH₂—CH₃), | A |
| B3 | | Benzaldehyde | | | 1.6(—CH₂—CH₂—CH₃), | |
| | | | | | 2.2-2.5(—CH₃), | |
| | | | | | 2.6(—CH₂—CH₂—CH₃), | |
| | | | | | 6.4(CH), 6.7-7.6(Ph—H), | |
| | | | | | 8.3-9.2(—OH) | |
| Example | RBiP-4 | ρ-(n-butyl) | 1276 | 1.22 | 0.9(—CH₂—CH₂—CH₂—CH₃), | A |
| B4 | | Benzaldehyde | | | 1.3(—CH₂—CH₂—CH₂—CH₃), | |
| | | | | | 1.6(—CH₂—CH₂—CH₂—CH₃), | |
| | | | | | 2.2-2.5(—CH₃), | |
| | | | | | 2.6(—CH₂—CH₂—CH₂—CH₃), | |
| | | | | | 6.4(CH), 6.7-7.6(Ph—H), 8.3~ | |
| | | | | | 9.2(—OH) | |
| Example | RBiP-5 | 4-Biphenyl- | 1325 | 1.45 | 2.2-2.5(—CH₃), 6.4(CH), 6.7- | A |
| B5 | | aldehyde | | | 7.6(Ph—H), 8.3-9.2(—OH) | |
| Example | RBiP-6 | 2-Naphth- | 998 | 1.30 | 2.2-2.5(—CH₃), 6.4(CH), 6.7- | A |
| B6 | | aldehyde | | | 7.6(Ph—H), 8.3~9.2(—OH) | |
| Example | RBiP-7 | 4-Hydroxy- | 1310 | 1.30 | 2.2-2.5 (—CH₃), 6.4(CH), 6.9- | A |
| B7 | | benzaldehyde | | | 7.6(Ph—H), 8.3-9.2(—OH) | |
| Example | RBiP-8 | Formaldehyde | 1610 | 1.40 | 2.2-2.5 (—CH₃), 6.4(CH), 6.7- | A |
| B8 | | | | | 7.6(Ph—H), 8.3-9.2(—OH) | |

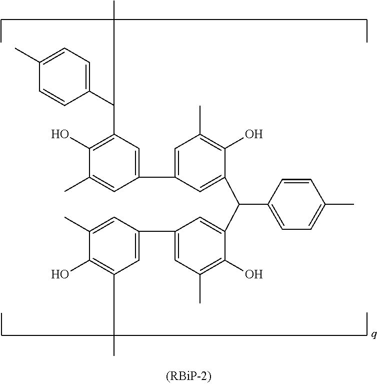

(In formula (RBiP-2), q represents the number of repeat units.) 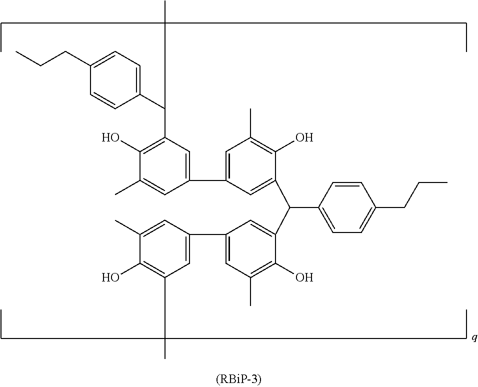
(In formula (RBiP-3), q represents the number of repeat units.) 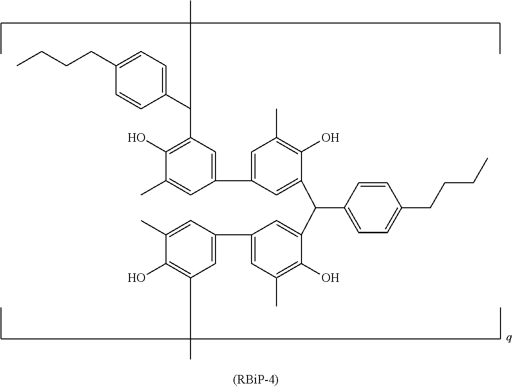
(In formula (RBiP-4), q represents the number of repeat units.) 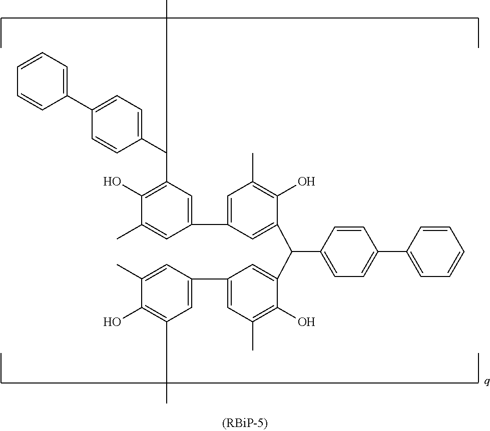
(In formula (RBiP-5), q represents the number of repeat units.) 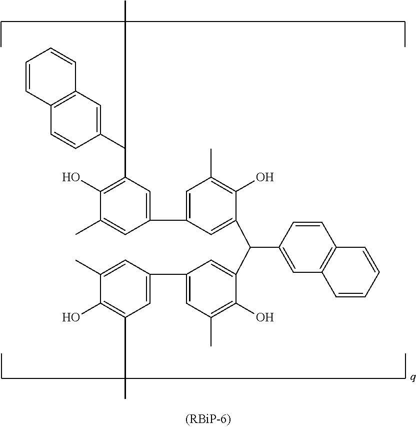
(In formula (RBiP-6), q represents the number of repeat units.) 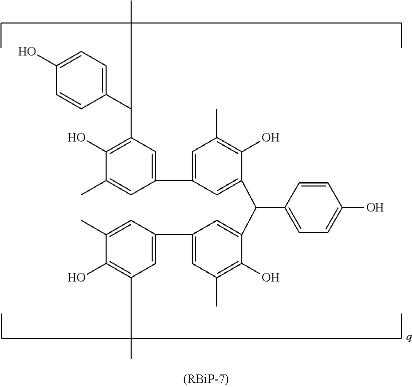
(In formula (RBiP-7), q represents the number of repeat units.) 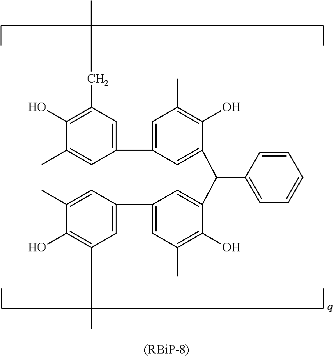
(In formula (RBiP-8), q represents the number of repeat units.)

### (Examples B9 to B11) Synthesis of Resins (RBiP-9), (RBiP-10), and (RBiP-11)

Objective resins (RBiP-9), (RBiP-10), and (RBiP-11) were obtained in the same manner as in Example B5 except that 3,3′-dimethylbiphenyl-4,4′-diol was changed to the raw materials shown in phenols of Table 4 below. The molecular weights, carbon concentrations, oxygen concentrations, and ¹H-NMR (500 MHz, DMSO-d₆) measurement results of the obtained compounds are shown in Table 4 below. The objective resins were confirmed to have chemical structures of the following formulae (RBiP-9), (RBiP-10), and (RBiP-11), respectively. Solubility test was conducted for the above resins (RBiP-9) to (RBiP-11) The results are shown in Table 4.

TABLE 4

| | | | GPC | | | |
|---|---|---|---|---|---|---|
| | Resin | Phenol species | Mw | Mw/Mn | ¹H-NMR | Solubility |
| Example | RBiP-9 | 2,2-Bis(4-hydroxy-3- | 1435 | 1.48 | 1.7(—CH₃), 2.1-2.2 | A |
| B9 | | methylphenyl)propane | | | (Ph—CH₃), 6.4(—CH), | |
| | | | | | 6.7-7.8(Ph—H), 8.3- | |
| | | | | | 9.2(—OH) | |
| Example | RBiP-10 | 4,4′-Methylenebis- | 1410 | 1.44 | 2.1-2.3(Ph—CH₃), | A |
| B10 | | (2,5-dimethylphenol) | | | 4.0(—CH₂—), | |
| | | | | | 6.4(—CH), 6.7- | |
| | | | | | 7.8(Ph—H), 8.3- | |
| | | | | | 9.2(—OH) | |
| Example | RBiP-11 | 3,3′-Diphenylbi- | 1520 | 1.50 | 6.5(—CH), 7.0-7.8 | A |
| B11 | | phenyl-4,4′-diol | | | (Ph—H), 8.3- | |
| | | | | | 9.2(—OH) | |

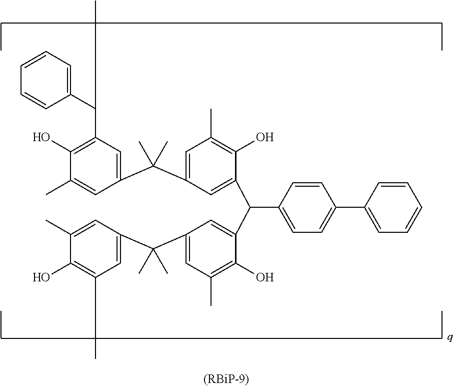

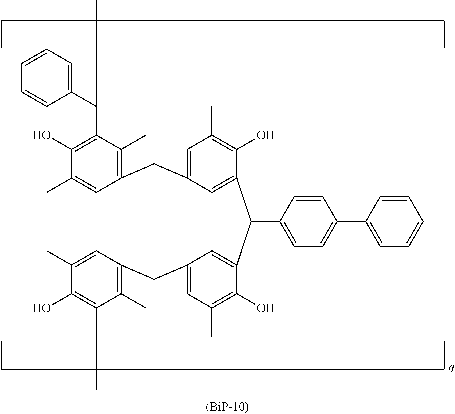

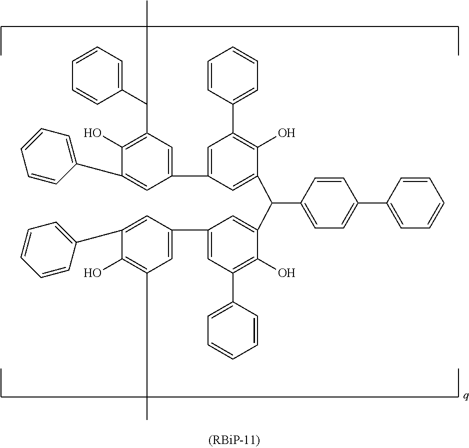

### Synthesis Comparative Example 1

A four necked flask (internal capacity: 10 L) equipped with a Dimroth condenser tube, a thermometer, and a stirring blade and having a detachable bottom was prepared. To this four necked flask, 1.09 kg (7 mol) of 1,5-dimethylnaphthalene (manufactured by Mitsubishi Gas Chemical Company, Inc.), 2.1 kg (28 mol as formaldehyde) of a 40 mass % aqueous formaldehyde solution (manufactured by Mitsubishi Gas Chemical Company, Inc.), and 0.97 ml of a 98 mass % sulfuric acid (manufactured by Kanto Chemical Co., Inc.) were added in a nitrogen stream, and the mixture was allowed to react for 7 hours while being refluxed at 100° C. at normal pressure. Subsequently, 1.8 kg of ethylbenzene (manufactured by Wako Pure Chemical Industries, Ltd., a special grade reagent) was added as a diluting solvent to the reaction liquid, and the mixture was left to stand still, followed by removal of an aqueous phase as a lower phase. Neutralization and washing with water were further performed, and ethylbenzene and unreacted 1,5-dimethylnaphthalene were distilled off under reduced pressure to obtain 1.25 kg of a dimethylnaphthalene formaldehyde resin as a light brown solid. The molecular weight of the obtained dimethylnaphthalene formaldehyde resin was as follows: number average molecular weight (Mn): 562, weight average molecular weight (Mw): 1168, and dispersity (Mw/Mn): 2.08.

Subsequently, a four necked flask (internal capacity: 0.5 L) equipped with a Dimroth condenser tube, a thermometer, and a stirring blade was prepared. To this four necked flask, 100 g (0.51 mol) of the dimethylnaphthalene formaldehyde resin obtained as described above and 0.05 g of p-toluenesulfonic acid were added in a nitrogen stream, and the temperature was raised to 190° C. at which the mixture was then heated for 2 hours, followed by stirring. Subsequently, 52.0 g (0.36 mol) of 1-naphthol was further added thereto, and the temperature was further raised to 220° C. at which the mixture was allowed to react for 2 hours. After dilution with a solvent, neutralization and washing with water were performed, and the solvent was distilled off under reduced pressure to obtain 126.1 g of resin (C-1) as a black-brown solid.

The obtained resin (C-1) had Mn: 885, Mw: 2220, and Mw/Mn: 2.51. In addition, the carbon concentration of the obtained resin (C-1) was 89.1% by mass, and the oxygen concentration thereof was 4.5% by mass.

Solubility test was conducted for the above resin (C-1). The evaluation result was A.

### Synthesis Comparative Example 2

A container (internal capacity: 200 mL) equipped with a stirrer, a condenser tube, and a burette was prepared. To this container, 30 g (161 mmol) of 4,4′-biphenol (a reagent manufactured by Tokyo Chemical Industry Co., Ltd.), 8.7 g (82 mmol) of benzaldehyde (a reagent manufactured by Tokyo Chemical Industry Co., Ltd.), and 100 mL of butyl acetate were added, and 3.9 g (21 mmol) of p-toluenesulfonic acid (a reagent manufactured by Kanto Chemical Co., Inc.) was added thereto to prepare a reaction liquid. This reaction liquid was stirred at 90° C. for 3 hours and reacted. Next, the reaction liquid was concentrated. The reaction product was precipitated by the addition of 50 g of heptane. After cooling to room temperature, the precipitates were separated by filtration. The solid matter obtained by filtration was dried and then separated and purified by column chromatography to obtain 4.2 g of the objective compound (C-2) represented by the following formula.

The following peaks were found by 400 MHz-1H-NMR, and the compound was confirmed to have a chemical structure of the following formula.

¹H-NMR: (DMSO-d₆, internal standard TMS)

δ (ppm) 9.4 (4H, O—H), 6.8-7.8 (19H, Ph-H), 6.2 (1H, C—H)

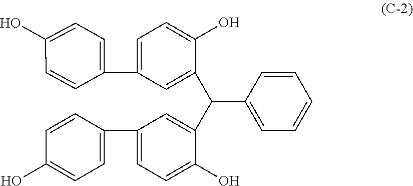 [SMILES: Oc1ccc(-c2ccc(O)c(C(c3ccccc3)c3cc(-c4ccc(O)cc4)ccc3O)c2)cc1]

### [Underlayer Film Formation]

### Examples C1 to C38 and Comparative Examples C1 and C2

Underlayer film forming materials (underlayer film forming compositions) were each prepared according to the composition shown in Table 5. Next, a silicon substrate was spin coated with each of these underlayer film forming materials, and then baked at 240° C. for 60 seconds and further at 400° C. for 120 seconds to prepare each underlayer film with a film thickness of 200 nm. The following acid generating agent, crosslinking agent, and organic solvent were used.

Acid generating agent: di-tert-butyl diphenyliodonium nonafluoromethanesulfonate (hereinafter, also referred to as “DTDPI”) (manufactured by Midori Kagaku Co., Ltd.)

Crosslinking agent: “NIKALAC MX270” (hereinafter, also referred to as “NIKALAC”) (product name, manufactured by Sanwa Chemical Co., Ltd.)

Organic solvent: propylene glycol monomethyl ether acetate (hereinafter, also referred to as “PGMEA”)

### [Evaluation of Etching Resistance]

For each of the obtained underlayer films, etching test was carried out under the following conditions to evaluate etching resistance according to the following method. The evaluation results are shown in Table 3.

### <Etching Test>

Etching apparatus: RIE-10NR (manufactured by Samco International, Inc.)

Output: 50 W

Pressure: 20 Pa

Time: 2 min

Etching gas

Ar gas flow rate:CF₄ gas flow rate:O₂ gas flow rate=50:5:5 (sccm)

### <Evaluation Method>

First, a novolac underlayer film used as the basis for evaluation was prepared according to the following method.

A novolac underlayer film was prepared under the same conditions as in Example C1 except that a phenol novolac resin “PSM4357” (product name, manufactured by Gunei Chemical Industry Co., Ltd.) was used instead of compound (BiP-1) used in Example C1. Then, the above etching test was conducted for this novolac underlayer film, and the etching rate (etching speed) at that time was measured. Next, for each of the underlayer films of Examples and Comparative Examples, the above etching test was conducted, and the etching rate at that time was measured. Then, the etching resistance for each of Examples and Comparative Examples was evaluated according to the following evaluation criteria on the basis of the etching rate of the underlayer film containing a phenol novolac resin.

<<Evaluation Criteria>>

A: The etching rate was less than −15% as compared with the underlayer film of novolac.

B: The etching rate was −15% to +5% as compared with the underlayer film of novolac.

C: The etching rate was more than +5% as compared with the underlayer film of novolac.

TABLE 5

| | Underlayer film forming material | | | | |
|---|---|---|---|---|---|
| | (Numerical values in parentheses are contents | | | | |
| | (parts by mass)) | | | | |
| | | | Acid | | |
| | Compound | | generating | Crosslinking | Etching |
| | or resin | Solvent | agent | agent | resistance |
| Example | BiP-1 | PGMEA | DTDPI | NIKALAC | A |
| C1 | (10) | (90) | (0.5) | (0.5) | |
| Example | BiP-2 | PGMEA | DTDPI | NIKALAC | A |
| C2 | (10) | (90) | (0.5) | (0.5) | |
| Example | BiP-3 | PGMEA | DTDPI | NIKALAC | A |
| C3 | (10) | (90) | (0.5) | (0.5) | |
| Example | BiP-4 | PGMEA | DTDPI | NIKALAC | A |
| C4 | (10) | (90) | (0.5) | (0.5) | |
| Example | BiP-5 | PGMEA | DTDPI | NIKALAC | A |
| C5 | (10) | (90) | (0.5) | (0.5) | |
| Example | BiP-6 | PGMEA | DTDPI | NIKALAC | A |
| C6 | (10) | (90) | (0.5) | (0.5) | |
| Example | RBiP-1 | PGMEA | DTDPI | NIKALAC | A |
| C7 | (10) | (90) | (0.5) | (0.5) | |
| Example | RBiP-2 | PGMEA | DTDPI | NIKALAC | A |
| C8 | (10) | (90) | (0.5) | (0.5) | |
| Example | RBiP-3 | PGMEA | DTDPI | NIKALAC | A |
| C9 | (10) | (90) | (0.5) | (0.5) | |
| Example | RBiP-4 | PGMEA | DTDPI | NIKALAC | A |
| C10 | (10) | (90) | (0.5) | (0.5) | |
| Example | RBiP-5 | PGMEA | DTDPI | NIKALAC | A |
| C11 | (10) | (90) | (0.5) | (0.5) | |
| Example | RBiP-6 | PGMEA | DTDPI | NIKALAC | A |
| C12 | (10) | (90) | (0.5) | (0.5) | |
| Example | BiP-1 | PGMEA | — | — | A |
| C13 | (10) | (90) | | | |
| Example | BiP-5 | PGMEA | — | — | A |
| C14 | (10) | (90) | | | |
| Example | RBiP-1 | PGMEA | — | — | A |
| C15 | (10) | (90) | | | |
| | Underlayer film forming material | | | | |
| | (Numerical values in parentheses are contents | | | | |
| | (parts by mass)) | | | | |
| | | | Acid | | |
| | Compound | | generating | Crosslinking | Etching |
| | or resin | Solvent | agent | agent | resistance |
| Example | RBiP-8 | PGMEA | DTDPI | NIKALAC | A |
| C16 | (10) | (90) | (9.5) | (0.5) | |
| Example | BiP-7 | PGMEA | DTDPI | NIKALAC | A |
| C17 | (10) | (90) | (0.5) | (0.5) | |
| Example | BiP-8 | PGMEA | DTDPI | NIKALAC | A |
| C18 | (10) | (90) | (0.5) | (0.5) | |
| Example | BiP-9 | PGMEA | DTDPI | NIKALAC | A |
| C19 | (10) | (90) | (0.5) | (0.5) | |
| Example | BiP-10 | PGMEA | DTDPI | NIKALAC | A |
| C20 | (10) | 90) | (0.5) | (0.5) | |
| Example | BiP-1- | PGMEA | DTDPI | NIKALAC | A |
| C21 | MeBOC | (90) | (0.5) | (0.5) | |
| | (10) | | | | |
| Example | BiP-1-BOC | PGMEA | DTDPI | NIKALAC | A |
| C22 | (10) | (90) | (0.5) | (0.5) | |
| Example | BiP-1-AL | PGMEA | DTDPI | — | A |
| C23 | (10) | (90) | (0.5) | | |
| Example | BiP-1-Ac | PGMEA | DTDPI | — | A |
| C24 | (10) | (90) | (0.5) | | |
| Example | BiP-1-Ea | PGMEA | DTDPI | — | A |
| C25 | (10) | (90) | (0.5) | | |
| Example | BiP-1-Ua | PGMEA | DTDPI | — | A |
| C26 | (10) | (90) | (0.5) | | |
| Example | BiP-1-E | PGMEA | DTDPI | — | A |
| C27 | (10) | (90) | (0.5) | | |
| Example | BiP-1-PX | PGMEA | DTDPI | — | A |
| C28 | (10) | (90) | (0.5) | | |
| Example | BiP-1-PE | PGMEA | DTDPI | — | A |
| C29 | (10) | (90) | (0.5) | | |
| Example | BiP-1-G | PGMEA | DTDPI | — | A |
| C30 | (10) | (90) | (0.5) | | |
| | Underlayer film forming material | | | | |
| | (Numerical values in parentheses are contents | | | | |
| | (parts by mass)) | | | | |
| | | | Acid | | |
| | Compound | | generating | Crosslinking | Etching |
| | or resin | Solvent | agent | agent | resistance |
| Example | BiP-1-GE | PGMEA | DTDPI | — | A |
| C31 | (10) | (90) | (0.5) | | |
| Example | BiP-1-SX | PGMEA | DTDPI | — | A |
| C32 | (10) | (90) | (0.5) | | |
| Example | BiP-1-SE | PGMEA | DTDPI | — | A |
| C33 | (10) | (90) | (0.5) | | |
| Example | BiP-1-Pr | PGMEA | DTDPI | — | A |
| C34 | (10) | (90) | (0.5) | | |
| Example | RBiP-7 | PGMEA | DTDPI | NIKALAC | A |
| C35 | (10) | (90) | (0.5) | (0.5) | |
| Example | RBiP-9 | PGMEA | DTDPI | NIKALAC | A |
| C36 | (10) | (90) | (0.5) | (0.5) | |
| Example | RBiP-10 | PGMEA | DTDPI | NIKALAC | A |
| C37 | (10) | (90) | (0.5) | (0.5) | |
| Example | RBiP-11 | PGMEA | DTDPI | NIKALAC | A |
| C38 | (10) | (90) | (0.5) | (0.5) | |
| Comparative | C-1 | PGMEA | DTDPI | NIKALAC | C |
| Example | (10) | (90) | (0.5) | (0.5) | |
| C1 | | | | | |
| Comparative | C-2 | PGMEA | DTDPI | NIKALAC | B |
| Example | (10) | (90) | (0.5) | (0.5) | |
| C2 | | | | | |

As clear from Table 5, in Examples C1 to C38 each using any of compounds (BiP-1) to (BiP-10), (BiP-1-MeBOC), (BiP-1-BOC), (BiP-1-AL), (BiP-1-Ac), (BiP-1-Ea), (BiP-1-Ua), (BiP-1-E), (BiP-1-PX), (BiP-1-PE), (BiP-1-G), (BiP-1-GE), (BiP-1-SX), (BiP-1-SE), (BiP-1-Pr), and resins (RBiP-1) to (RBiP-11) of the present embodiment, both of solubility and etching resistance are confirmed to be excellent. On the other hand, Comparative Example C1 using resin (C-1) (phenol-modified dimethylnaphthaleneformaldehyde resin) resulted in poor etching resistance.

### [Underlayer Film Formation]

### Examples D1 to D38

A SiO₂ substrate with a film thickness of 300 nm was coated with the solution of the underlayer film forming material prepared in each of the above Examples C1 to C38, and baked at 240° C. for 60 seconds and further at 400° C. for 120 seconds to form each underlayer film with a film thickness of 70 nm. This underlayer film was coated with a resist solution for ArF and baked at 130° C. for 60 seconds to form a photoresist film with a film thickness of 140 nm. The ArF resist solution used was prepared by compounding 5 parts by mass of a resin represented by the following formula (11), 1 part by mass of triphenylsulfonium nonafluoromethanesulfonate, 2 parts by mass of tributylamine, and 92 parts by mass of PGMEA. For the resin represented by the following formula (11), 4.15 g of 2-methyl-2-methacryloyloxyadamantane, 3.00 g of methacryloyloxy-γ-butyrolactone, 2.08 g of 3-hydroxy-1-adamantyl methacrylate, and 0.38 g of azobisisobutyronitrile were dissolved in 80 mL of tetrahydrofuran to prepare a reaction solution. This reaction solution was polymerized for 22 hours with the reaction temperature kept at 63° C. in a nitrogen atmosphere. Then, the reaction solution was added dropwise into 400 mL of n-hexane. The product resin thus obtained was solidified and purified, and the white powder produced was filtered and dried overnight at 40° C. under reduced pressure to obtain a compound.

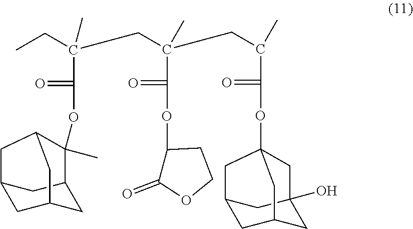 [SMILES: CCC(C)(CCC(C)(CCC(C)C(=O)OC12CC3CC(CC(O)(C3)C1)C2)C(=O)OC1CCOC1=O)C(=O)OC1(C)C2CC3CC(C2)CC1C3]

(The numbers in formula (11) indicate the ratios of respective constituent units.)

Subsequently, the photoresist film was exposed using electron beam lithography system “ELS-7500” (product name, manufactured by ELIONIX INC., 50 keV), baked (PEB) at 115° C. for 90 seconds, and developed for 60 seconds in a 2.38 mass tetramethylammonium hydroxide (TMAH) aqueous solution to obtain a positive type resist pattern.

Defects of the obtained resist patterns of 55 nm L/S (1:1) and 80 nm L/S (1:1) were observed, and the results are shown in Table 4.

In the table, “good” means that no major defects were found in the formed resist pattern, and “poor” means that major defects were found in the formed resist pattern.

### Comparative Example D1

The same operations as in Example D1 were carried out except that no underlayer film was formed so that a photoresist film was formed directly on a SiO₂ substrate to obtain a positive type resist pattern. The results are shown in Table 6.

TABLE 6

| | Underlayer film | | | Resist pattern |
|---|---|---|---|---|
| | forming | Resolution | Sensitivity | shape after |
| | material | (nmL/S) | (μC/cm²) | development |
| ExampleD1 | ExampleC1 | 55 | 10 | Good |
| ExampleD2 | ExampleC2 | 55 | 10 | Good |
| ExampleD3 | ExampleC3 | 55 | 10 | Good |
| ExampleD4 | ExampleC4 | 55 | 10 | Good |
| ExampleD5 | ExampleC5 | 55 | 10 | Good |
| ExampleD6 | ExampleC6 | 55 | 10 | Good |
| ExampleD7 | ExampleC7 | 55 | 10 | Good |
| ExampleD8 | ExampleC8 | 55 | 10 | Good |
| ExampleD9 | ExampleC9 | 55 | 10 | Good |
| ExampleD10 | ExampleC10 | 55 | 10 | Good |
| ExampleD11 | ExampleC11 | 55 | 10 | Good |
| ExampleD12 | ExampleC12 | 55 | 10 | Good |
| ExampleD13 | ExampleC13 | 55 | 10 | Good |
| ExampleD14 | ExampleC14 | 55 | 10 | Good |
| ExampleD15 | ExampleC15 | 55 | 10 | Good |
| ExampleD16 | ExampleC16 | 55 | 10 | Good |
| ExampleD17 | ExampleC17 | 55 | 10 | Good |
| ExampleD18 | ExampleC18 | 55 | 10 | Good |
| ExampleD19 | ExampleC19 | 55 | 10 | Good |
| ExampleD20 | ExampleC20 | 55 | 10 | Good |
| ExampleD21 | ExampleC21 | 55 | 10 | Good |
| ExampleD22 | ExampleC22 | 55 | 10 | Good |
| ExampleD23 | ExampleC23 | 55 | 10 | Good |
| ExampleD24 | ExampleC24 | 55 | 10 | Good |
| ExampleD25 | ExampleC25 | 55 | 10 | Good |
| ExampleD26 | ExampleC26 | 55 | 10 | Good |
| ExampleD27 | ExampleC27 | 55 | 10 | Good |
| ExampleD28 | ExampleC28 | 55 | 10 | Good |
| ExampleD29 | ExampleC29 | 55 | 10 | Good |
| ExampleD30 | ExampleC30 | 55 | 10 | Good |
| | Underlayer film | | | Resist pattern |
| | forming | Resolution | Sensitivity | shape after |
| | material | (nmL/S) | (μC/cm²) | development |
| ExampleD31 | ExampleC31 | 55 | 10 | Good |
| ExampleD32 | ExampleC32 | 55 | 10 | Good |
| ExampleD33 | ExampleC33 | 55 | 10 | Good |
| ExampleD34 | ExampleC34 | 55 | 10 | Good |
| ExampleD35 | ExampleC35 | 55 | 10 | Good |
| ExampleD36 | ExampleC36 | 55 | 10 | Good |
| ExampleD37 | ExampleC37 | 55 | 10 | Good |
| ExampleD38 | ExampleC38 | 55 | 10 | Good |
| Comparative | — | 80 | 26 | Poor |
| ExampleD1 | | | | |

As clear from Table 6, in Examples D1 to D38 each using any of compounds (BiP-1) to (BiP-10), (BiP-1-MeBOC), (BiP-1-BOC), (BiP-1-AL), (BiP-1-Ac), (BiP-1-Ea), (BiP-1-Ua), (BiP-1-E), (BiP-1-PX), (BiP-1-PE), (BiP-1-G), (BiP-1-GE), (BiP-1-SX), (BiP-1-SE), (BiP-1-Pr), and resins (RBiP-1) to (RBiP-11) of the present embodiment, it was confirmed that the resist pattern shapes after development were good, and major defects were not found. Furthermore, each of Examples D1 to D38 was confirmed to be significantly superior to Comparative Example D1, in which no underlayer film was formed, in both resolution and sensitivity. Here, a good resist pattern shape after development indicates that the underlayer film forming materials used in Examples D1 to D38 have good adhesiveness to a resist material (photoresist material and the like).

### [Underlayer Film Formation]

### Examples E1 to E38

A SiO₂ substrate with a film thickness of 300 nm was coated with the solution of the underlayer film forming material for lithography according to each of Examples C1 to C38, and baked at 240° C. for 60 seconds and further at 400° C. for 120 seconds to form each underlayer film with a film thickness of 80 nm. This underlayer film was coated with a silicon-containing intermediate layer material and baked at 200° C. for 60 seconds to form an intermediate layer film with a film thickness of 35 nm. This intermediate layer film was further coated with the above resist solution for ArF and baked at 130° C. for 60 seconds to form a photoresist film with a film thickness of 150 nm. The silicon-containing intermediate layer material used was the silicon atom-containing polymer described in <Synthesis Example 1> of Japanese Patent Laid-Open No. 2007-226170. Subsequently, the photoresist film was mask exposed using an electron beam lithography system (manufactured by ELIONIX INC.; ELS-7500, 50 keV), baked (PEB) at 115° C. for 90 seconds, and developed for 60 seconds in a 2.38 mass % tetramethylammonium hydroxide (hereinafter, also referred to as “TMAH”) aqueous solution to obtain a 55 nm L/S (1:1) positive type resist pattern. Thereafter, the silicon-containing intermediate layer film (hereinafter, also referred to as “SOG”) was dry etched with the obtained resist pattern as a mask using parallel plate RIE system “RIE-10NR” (product name, manufactured by Samco International, Inc.). Subsequently, dry etching of the underlayer film with the obtained silicon-containing intermediate layer film pattern as a mask and dry etching of the SiO₂ film with the obtained underlayer film pattern as a mask were performed in order.

Respective etching conditions are as shown below.

Conditions for Etching of Resist Intermediate Layer Film with Resist Pattern

Output: 50 W

Pressure: 20 Pa

Time: 1 min

Etching gas

Ar gas flow rate:CF₄ gas flow rate:O gas flow rate=50:8:2 (sccm)

Conditions for Etching of Underlayer Film with Resist Intermediate Film Pattern

Output: 50 W

Pressure: 20 Pa

Time: 2 min

Etching gas

Ar gas flow rate:CF₄ gas flow rate:O₂ gas flow rate=50:5:5 (sccm)

Conditions for Etching of SiO₂ Film with Underlayer Film Pattern

Output: 50 W

Pressure: 20 Pa

Time: 2 min

Etching gas

Ar gas flow rate:C₅F₁₂ gas flow rate:C₂F₆ gas flow rate:O₂ gas flow rate

=50:4:3:1 (sccm)

### [Resist Pattern Formability Evaluation]

The pattern cross section (that is, the shape of the SiO₂ film after etching) obtained as described above was observed by using an electron microscope “S-4800” (product name, manufactured by Hitachi, Ltd.) to evaluate resist pattern formability. The observation results are shown in Table 5. In the table, “good” means that no major defects were found in the formed pattern cross section, and “poor” means that major defects were found in the formed pattern cross section. The evaluation results are shown in Table 7.

TABLE 7

| | Underlayer film | Shape of | Resist |
|---|---|---|---|
| | forming | SiO2 | pattern |
| | material | film | formability |
| ExampleE1 | ExampleC1 | 10 | Good |
| ExampleE2 | ExampleC2 | 10 | Good |
| ExampleE3 | ExampleC3 | 10 | Good |
| ExampleE4 | ExampleC4 | 10 | Good |
| ExampleE5 | ExampleC5 | 10 | Good |
| ExampleE6 | ExampleC6 | 10 | Good |
| ExampleE7 | ExampleC7 | 10 | Good |
| ExampleE8 | ExampleC8 | 10 | Good |
| ExampleE9 | ExampleC9 | 10 | Good |
| ExampleE10 | ExampleC10 | 10 | Good |
| ExampleE11 | ExampleC11 | 10 | Good |
| ExampleE12 | Exam leC12 | 10 | Good |
| ExampleE13 | ExampleC13 | 10 | Good |
| ExampleE14 | ExampleC14 | 10 | Good |
| ExampleE15 | ExampleC15 | 10 | Good |
| ExampleE16 | ExampleC16 | 10 | Good |
| ExampleE17 | ExampleC17 | 10 | Good |
| ExampleE18 | ExampleC18 | 10 | Good |
| ExampleE19 | ExampleC19 | 10 | Good |
| ExampleE20 | ExampleC20 | 10 | Good |
| ExampleE21 | ExampleC21 | 10 | Good |
| ExampleE22 | ExampleC22 | 10 | Good |
| ExampleE23 | ExampleC23 | 10 | Good |
| ExampleE24 | ExampleC24 | 10 | Good |
| ExampleE25 | ExampleC25 | 10 | Good |
| ExampleE26 | ExampleC26 | 10 | Good |
| ExampleE27 | ExampleC27 | 10 | Good |
| ExampleE28 | ExampleC28 | 10 | Good |
| ExampleE29 | ExampleC29 | 10 | Good |
| ExampleE30 | ExampleC30 | 10 | Good |
| | Underlayer film | Shape of | Resist |
| | forming | SiO2 | pattern |
| | material | film | formability |
| ExampleE31 | ExampleC31 | 10 | Good |
| ExampleE32 | ExampleC32 | 10 | Good |
| ExampleE33 | ExampleC33 | 10 | Good |
| ExampeE34 | ExampleC34 | 10 | Good |
| ExampleE35 | ExampleC35 | 10 | Good |
| ExampleE36 | ExampleC36 | 10 | Good |
| ExampleE37 | ExampleC37 | 10 | Good |
| ExampleE38 | ExampleC38 | 10 | Good |

### [Optical Article Formation]

### Examples F1 to F38

A SiO₂ substrate with a film thickness of 300 nm was coated with an optical component forming composition solution having the same composition as that of the solution of the underlayer film forming material for lithography prepared in each of the above Examples C1 to C38, and baked at 260° C. for 300 seconds to form each optical component forming film with a film thickness of 100 nm. The refractive index and transparency were evaluated according to the following method. The evaluation results are shown in Table 8.

### [Refractive Index and Transparency Tests]

Tests for the refractive index and the transparency at a wavelength of 633 nm were carried out by using vacuum ultraviolet with variable angle spectroscopic ellipsometer “VUV-VASE” (product name, manufactured by J.A. Woollam Japan), and the refractive index and the transparency were evaluated according to the following criteria.

### <<Evaluation Criteria for Refractive Index>>

A: The refractive index is 1.60 or more.

C: The refractive index is less than 1.60.

### <<Evaluation Criteria for Transparency>>

A: The absorption coefficient is less than 0.03.

C: The absorption coefficient is 0.03 or more.

TABLE 8

| | Underlayer | | |
|---|---|---|---|
| | film forming | Refractive | |
| | material | index | Transparency |
| ExampleF1 | ExampleC1 | A | A |
| ExampleF2 | ExampleC2 | A | A |
| ExampleF3 | ExampleC3 | A | A |
| ExampleF4 | ExampleC4 | A | A |
| ExampleF5 | ExampleC5 | A | A |
| ExampleF6 | ExampleC6 | A | A |
| ExampleF7 | ExampleC7 | A | A |
| ExampleF8 | ExampleC8 | A | A |
| ExampleF9 | ExampleC9 | A | A |
| ExampleF10 | ExampleC10 | A | A |
| ExampleF11 | ExampleC11 | A | A |
| ExampleF12 | ExampleC12 | A | A |
| ExampleF13 | ExampleC13 | A | A |
| ExampleF14 | ExampleC14 | A | A |
| ExampleF15 | ExampleC15 | A | A |
| ExampleF16 | ExampleC16 | A | A |
| ExampleF17 | ExampleC17 | A | A |
| ExampleF18 | ExampleC18 | A | A |
| ExampleF19 | ExampleC19 | A | A |
| ExampleF20 | ExampleC20 | A | A |
| ExampleF21 | ExampleC21 | A | A |
| ExampleF22 | ExampleC22 | A | A |
| ExampleF23 | ExampleC23 | A | A |
| ExampleF24 | ExampleC24 | A | A |
| ExampleF25 | ExampleC25 | A | A |
| ExampleF26 | ExampleC26 | A | A |
| ExampleF27 | ExampleC27 | A | A |
| ExampleF28 | ExampleC28 | A | A |
| ExampleF29 | ExampleC29 | A | A |
| ExampleF30 | ExampleC30 | A | A |
| | Underlayer | | |
| | film forming | Refractive | |
| | material | index | Transparency |
| ExampleF31 | ExampleC31 | A | A |
| ExampleF32 | ExampleC32 | A | A |
| ExampleF33 | ExampleC33 | A | A |
| ExampleF34 | ExampleC34 | A | A |
| ExampleF35 | ExampleC35 | A | A |
| ExampleF36 | ExampleC36 | A | A |
| ExampleF37 | ExampleC37 | A | A |
| ExampleF38 | ExampleC38 | A | A |

### [Purification Method]

### (Example G1) Purification of Compound (BiP-1) by Acid

To a four necked flask (capacity: 1000 mL, with a detachable bottom), 150 g of a solution (10% by mass) formed by dissolving compound (BiP-1) obtained in Example A1 in PGMEA was added, and was heated to 80° C. with stirring. Then, 37.5 g of an aqueous oxalic acid solution (pH 1.3) was added thereto, and the resultant mixture was stirred for 5 minutes and then left to stand still for 30 minutes. This separated the mixture into an oil phase and an aqueous phase, and the aqueous phase was thus removed. After repeating this operation once, 37.5 g of ultrapure water was added to the obtained oil phase, and after stirring for 5 minutes, the mixture was left to stand for 30 minutes, and the aqueous phase was removed. After repeating this operation three times, the residual water and PGMEA were concentrated and removed by heating to 80° C. and reducing the pressure in the flask to 200 hPa or less. Thereafter, by diluting with PGMEA of EL grade (a reagent manufactured by Kanto Chemical Co., Inc.) such that the concentration was adjusted to 10 by mass, a PGMEA solution of BiP-1 with a reduced metal content was obtained.

### (Comparative Example G1) Purification of Compound (BiP-1) by Ultrapure Water

In the same manner as of Example G1 except that ultrapure water was used instead of the aqueous oxalic acid solution, and by adjusting the concentration to 10% by mass, a PGMEA solution of compound (BiP-1) was obtained.

For the 10 mass % PGMEA solution of compound (BiP-1) before the treatment, and the solutions obtained in Example G1 and Comparative Example G1, the contents of various metals were measured by ICP-MS. The measurement results are shown in Table 9.

TABLE 9

| | Metal content (ppb) | | | | | |
|---|---|---|---|---|---|---|
| | Na | Mg | K | Fe | Cu | Zn |
| Compound | >99 | 22.2 | >99 | >99 | 11.5 | 10.0 |
| before | | | | | | |
| treatment | | | | | | |
| (BiP-1) | >99 | | | | | |
| ExampleG1 | 1.8 | 1.1 | 0.5 | 1.6 | 0.2 | 0.2 |
| Comparative | 2.5 | 1.5 | 1.2 | >99 | 2.3 | 3.3 |
| ExampleG1 | | | | | | |

### [Resist Film Formation]

### Examples H1 to H38 and Comparative Examples H1 and H²

Resist film forming materials (resist film forming compositions) were each prepared according to the composition shown in Table 10. Thereafter, a clean silicon wafer was spin coated with a homogeneous resist film forming composition, and then pre-exposure baked (PB) in an oven at 110° C. to form a resist film with a thickness of 60 nm. The obtained resist film was irradiated with electron beams of 1:1 line and space setting with 50 nm, 40 nm, and 30 nm intervals using electron beam lithography system “ELS-7500” (product name, manufactured by ELIONIX INC.). After the irradiation, the resist film was heated at each predetermined temperature for 90 seconds, and immersed in PGME for 60 seconds for development. Subsequently, the resist film was washed with ultrapure water for 30 seconds, and dried to form a negative type resist pattern.

### [Sensitivity and Pattern Formability Tests]

Concerning the formed resist pattern, the line and space were observed by scanning electron microscope “S-4800” (product name, manufactured by Hitachi High-Technologies Corporation) to evaluate the reactivity by electron beam irradiation of the resist film forming composition. The results thereof are shown in Table 10.

### <<Evaluation Criteria for Sensitivity>>

The sensitivity was indicated by the smallest energy quantity per unit area necessary for obtaining patterns, and evaluated according to the following criteria.

A: the pattern was obtained at less than 40 μC/cm².

C: the pattern was obtained at 40 μC/cm² or more.

### <<Evaluation Criteria for Pattern Formation>>

As for pattern formation, the obtained pattern shape was observed under scanning electron microscope (SEM) “S-4800” (product name, manufactured by Hitachi High-Technologies Corporation), and evaluated according to the following criteria.

A: a rectangular pattern without residues was obtained.

B: an almost rectangular pattern with almost no residues was obtained.

C: a non-rectangular pattern was obtained.

In Table 10, the following acid generating agent, crosslinking agent, and organic solvent were used.

Acid generating agent: triphenylbenzenesulfonium trifluoromethanesulfonate (hereinafter, also referred to as “TPS”)

Crosslinking agent: “NIKALAC MW-100LM” (hereinafter, also referred to as “NIKALAC MW”) (product name, manufactured by Sanwa Chemical Co., Ltd.)

Organic solvent: propylene glycol monomethyl ether acetate (hereinafter, also referred to as “PGMEA”)

Acid diffusion controlling agent: trioctylamine (hereinafter, also referred to as “TOA”)

TABLE 10

| | Resist film forming material | | | | | | |
|---|---|---|---|---|---|---|---|
| | (Numerical values in parentheses are contents (parts by mass)) | | | | | | |
| | | | | | Acid | | |
| | | | Acid | | diffusion | | |
| | Compound | | generating | Crosslinking | controlling | | Pattern |
| | or resin | Solvent | agent | agent | agent | Sensitivity | shape |
| Example | BiP-1 | PGMEA | TPS | NIKALACMW | TOA | A | A |
| H1 | (10) | (300) | (3) | (3) | (0.3) | | |
| Example | BiP-2 | PGMEA | TPS | NIKALACMW | TOA | A | A |
| H2 | (10) | (300) | (3) | (3) | (0.3) | | |
| Example | BiP-3 | PGMEA | TPS | NIKALACMW | TOA | A | A |
| H3 | (10) | (300) | (3) | (3) | (0.3) | | |
| Example | BiP-4 | PGMEA | TPS | NIKALACMW | TOA | A | A |
| H4 | (10) | (300) | (3) | (3) | (0.3) | | |
| Example | BiP-5 | PGMEA | TPS | NIKALACMW | TOA | A | A |
| H5 | (10) | (300) | (3) | (3) | (0.3) | | |
| Example | BiP-6 | PGMEA | TPS | NIKALACMW | TOA | A | A |
| H6 | (10) | (300) | (3) | (3) | (0.3) | A | |
| Example | RBiP-1 | PGMEA | TPS | NIKALACMW | TOA | A | A |
| H7 | (10) | (300) | (3) | (3) | (0.3) | | |
| Example | RBiP-2 | PGMEA | TPS | NIKALACMW | TOA | A | A |
| H8 | (10) | (300) | (3) | (3) | (0.3) | | |
| Example | RBiP-3 | PGMEA | TPS | NIKALACMW | TOA | A | A |
| H9 | (10) | (300) | (3) | (3) | (0.3) | | |
| Example | RBiP-4 | PGMEA | TPS | NIKALACMW | TOA | A | A |
| H10 | (10) | (300) | (3) | (3) | (0.3) | | |
| Example | RBiP-5 | PGMEA | TPS | NIKALACMW | TOA | A | A |
| H11 | (10) | (300) | (3) | (3) | (0.3) | | |
| Example | RBiP-6 | PGMEA | TPS | NIKALACMW | TOA | A | A |
| H12 | (10) | (300) | (3) | (3) | (0.3) | | |
| Example | BiP-1 | PGMEA | — | — | — | C | A |
| H13 | (10) | (300) | | | | | |
| Example | BiP-5 | PGMEA | — | — | — | C | A |
| H14 | (10) | (300) | | | | | |
| Example | RBiP-1 | PGMEA | — | — | — | C | A |
| H15 | (10) | (300) | | | | | |
| | Resist film forming material | | | | | | |
| | (Numerical values in parentheses are contents (parts by mass)) | | | | | | |
| | | | | | Acid | | |
| | | | Acid | | diffusion | | |
| | Compound | | generating | Crosslinking | controlling | | Pattern |
| | or resin | Solvent | agent | agent | agent | Sensitivity | shape |
| Example | RBiP-8 | PGMEA | TPS | NIKALACMW | TOA | A | A |
| H16 | (10) | (300) | (3) | (3) | (0.3) | | |
| Example | BiP-7 | PGMEA | TPS | NIKALACMW | TOA | A | A |
| H17 | (10) | (300) | (3) | (3) | (0.3) | | |
| Example | BiP-8 | PGMEA | TPS | NIKALACMW | TOA | A | A |
| H18 | (10) | (300) | (3) | (3) | (0.3) | | |
| Example | BiP-9 | PGMEA | TPS | NIKALACMW | TOA | A | A |
| H19 | (10) | (300) | (3) | (3) | (0.3) | | |
| Example | BiP-10 | PGMEA | TPS | NIKALACMW | TOA | A | A |
| H20 | (10) | (300) | (3) | (3) | (0.3) | | |
| Example | RBiP-7 | PGMEA | TPS | NIKALACMW | TOA | A | A |
| H21 | (10) | (300) | (3) | (3) | (0.3) | | |
| Example | RBiP-9 | PGMEA | TPS | NIKALACMW | TOA | A | A |
| H22 | (10) | (300) | (3) | (3) | (0.3) | | |
| Example | RBiP-10 | PGMEA | TPS | NIKALACMW | TOA | A | A |
| H23 | (10) | (300) | (3) | (3) | (0.3) | | |
| Example | RBiP-11 | PGMEA | TPS | NIKALACMW | TOA | A | A |
| H24 | (10) | (300) | (3) | (3) | (0.3) | | |
| Example | BiP-1- | PGMEA | TPS | NIKALACMW | TOA | A | A |
| H25 | MeBOC | (300) | (3) | (3) | (0.3) | | |
| | (10) | | | | | | |
| Example | BiP-1-BOC | PGMEA | TPS | NIKALACMW | TOA | A | A |
| H26 | (10) | (300) | (3) | (3) | (0.3) | | |
| Example | BiP-1-AL | PGMEA | TPS | — | TOA | A | A |
| H27 | (10) | (300) | (3) | | (0.3) | | |
| Example | BiP-1-Ac | PGMEA | TPS | — | TOA | A | A |
| H28 | (10) | (300) | (3) | | (0.3) | | |
| Example | BiP-1-Ea | PGMEA | TPS | — | TOA | A | A |
| H29 | (10) | (300) | (3) | | (0.3) | | |
| Example | BiP-1-Ua | PGMEA | TPS | — | TOA | A | A |
| H30 | (10) | (300) | | | (0.3) | | |
| | Resist film forming material | | | | | | |
| | (Numerical values in parentheses are contents (parts by mass)) | | | | | | |
| | | | | | Acid | | |
| | | | Acid | | diffusion | | |
| | Compound | | generating | Crosslinking | controlling | | Pattern |
| | or resin | Solvent | agent | agent | agent | Sensitivity | shape |
| Example | BiP-1-E | PGMEA | TPS | — | TOA | A | A |
| H31 | (10) | (300) | (3) | | (0.3) | | |
| Example | BiP-1-PX | PGMEA | TPS | — | TOA | A | A |
| H32 | (10) | (300) | (3) | | (0.3) | | |
| Example | BiP-1-PE | PGMEA | TPS | — | TOA | A | A |
| H33 | (10) | (300) | (3) | | (0.3) | | |
| Example | BiP-1-G | PGMEA | TPS | — | TOA | A | A |
| H34 | (10) | (300) | (3) | | (0.3) | | |
| Example | BiP-1-GE | PGMEA | TPS | — | TOA | A | A |
| H35 | (10) | (300) | (3) | | (0.3) | | |
| Example | BiP-1-SX | PGMEA | TPS | — | TOA | A | A |
| H36 | (10) | (300) | (3) | | (0.3) | | |
| Example | BiP-1-SE | PGMEA | FPS | — | TOA | A | A |
| H37 | (10) | (300) | (3) | | (0.3) | | |
| Example | BiP-1-Pr | PGMEA | TPS | — | TOA | A | A |
| H38 | (10) | (300) | (3) | | (0.3) | | |
| Comparative | C-1 | PGMEA | DTDPI | NIKALACMW | TOA | C | C |
| Example H1 | (10) | (300) | (3) | (3) | (0.3) | | |
| Comparative | C-2 | PGMEA | DTDPI | NIKALACMW | TOA | C | B |
| Example H2 | (10) | (300) | (3) | (3) | (0.3) | | |

### INDUSTRIAL APPLICABILITY

The compound and the resin of the present invention has high heat resistance, has high solvent solubility, and is applicable to a wet process. Therefore, a film forming material for lithography using the compound or the resin of the present invention, and a film for lithography thereof can be utilized widely and effectively in various applications that require such performances. Accordingly, the present invention can be utilized widely and effectively in, for example, electrical insulating materials, resins for resists, encapsulation resins for semiconductors, adhesives for printed circuit boards, electrical laminates mounted in electric equipment, electronic equipment, industrial equipment, and the like, matrix resins of prepregs mounted in electric equipment, electronic equipment, industrial equipment, and the like, buildup laminate materials, resins for fiber-reinforced plastics, resins for encapsulation of liquid crystal display panels, coating materials, various coating agents, adhesives, coating agents for semiconductors, resins for resists for semiconductors, resins for underlayer film formation, and the like. In particular, the present invention can be utilized particularly effectively in the field of films for lithography.

1. A compound represented by the following formula (1): 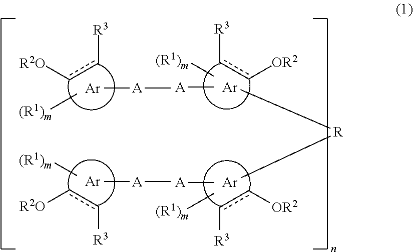
wherein
each A is independently a single bond or a linking group;
Ar is an aromatic ring;
R is a 2n-valent group having 1 to 60 carbon atoms and optionally having a substituent and/or a heteroatom;
each R¹ is independently a linear, branched, or cyclic alkyl group having 1 to 30 carbon atoms, an aryl group having 6 to 40 carbon atoms, an alkenyl group having 2 to 30 carbon atoms, an alkynyl group having 2 to 30 carbon atoms, a halogen atom, a nitro group, an amino group, a carboxy group, a cyano group, a mercapto group, or a hydroxy group;
each R² is independently a hydrogen atom, a crosslinkable group, a dissociable group, a linear, branched, or cyclic alkyl group having 1 to 30 carbon atoms, or an aryl group having 6 to 40 carbon atoms;
provided that at least one R² is any of a hydrogen atom, a crosslinkable group, and a dissociable group;
each R³ is independently a linear, branched, or cyclic alkyl group having 1 to 30 carbon atoms, an aryl group having 6 to 40 carbon atoms, an alkenyl group having 2 to 30 carbon atoms, an alkynyl group having 2 to 30 carbon atoms, a halogen atom, a nitro group, an amino group, a carboxy group, a cyano group, a mercapto group, or a hydroxy group;
each m is independently an integer of 0 to 8;
n is an integer of 1 to 4; and
the alkyl group, the aryl group, the alkenyl group, and the alkynyl group optionally have a substituent and/or a heteroatom,
provided that a compound represented by the following formula (A) is excluded:

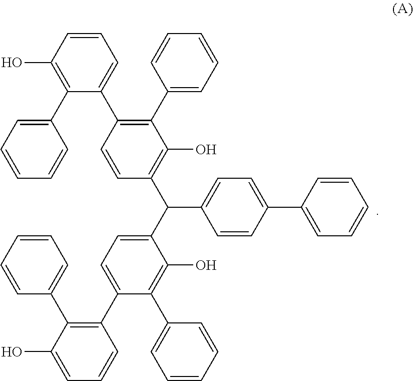 [SMILES: Oc1cccc(-c2ccc(C(c3ccc(-c4ccccc4)cc3)c3ccc(-c4cccc(O)c4-c4ccccc4)c(-c4ccccc4)c3O)c(O)c2-c2ccccc2)c1-c1ccccc1]

2. The compound according to claim 1, represented by the following formula (1-1): 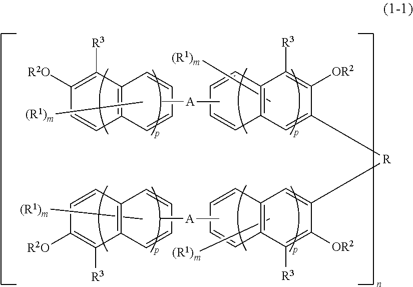
wherein
A, R, R¹ to R³, n, and m are each as defined in the above formula (1); and
each p is independently an integer of 0 to 3.

3. The compound according to claim 2, wherein each R² is independently a hydrogen atom, a linear, branched, or cyclic alkyl group having 1 to 30 carbon atoms, or an aryl group having 6 to 40 carbon atoms; and at least one R² is a hydrogen atom.

4. The compound according to claim 2, wherein when p is 0, a substitution position of A is a para position with respect to the R²O— group.

5. The compound according to claim 1, wherein the compound represented by the formula (1) is a compound represented by the following formula (1a): 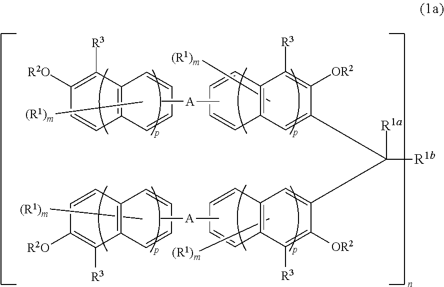
wherein
A, R¹ to R³, n, m, and p are each as defined in the formula (1) or the formula (1-1);
R^{1a} is a hydrogen atom or a monovalent group having 1 to 10 carbon atoms;
R^{1b} is an n-valent group having 1 to 30 carbon atoms;
R^{1a} and R^{1b} may bind to each other to form a cyclic group having 2 to 40 carbon atoms; and
the monovalent group and the n-valent group optionally have a substituent and/or a heteroatom.

6. The compound according to claim 5, wherein the compound represented by the formula (1a) is a compound represented by the following formula (1b): 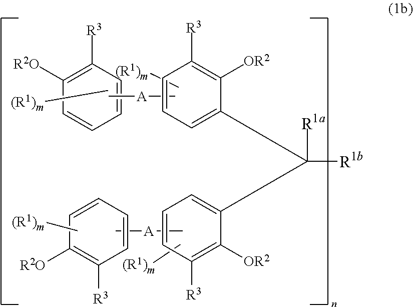
wherein
A, R¹ to R³, R^{1a}, R^{1b}, n, and m are each as defined in the formula (1) or the formula (1a).

7. The compound according to claim 6, wherein the compound represented by the formula (1b) is a compound represented by the following formula (1c): 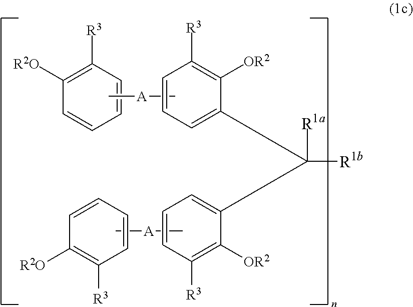
wherein
A, R², R³, R^{1a}, R^{1b}, and n are each as defined in the formula (1) or the formula (1a).

8. (canceled)

9. (canceled)

10. The compound according to claim 6, wherein the compound represented by the formula (1b) is a compound represented by the following formula (1d-1): 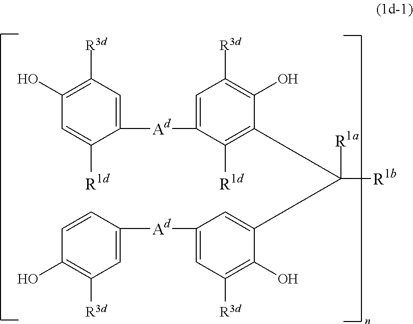
wherein
R^{1a}, R^{1b}, and n are each as defined in the formula (1) or the formula (1a); each Rad is independently a linear or branched alkyl group having 1 to 4 carbon atoms or a phenyl group; each R^{1d} is independently a hydrogen atom or a linear or branched alkyl group having 1 to 4 carbon atoms; and A^{d} is a single bond, a methylene group, or a 2,2-propanediyl group.

11. (canceled)

12. (canceled)

13. (canceled)

14. (canceled)

15. (canceled)

16. (canceled)

17. (canceled)

18. A resin containing a constituent unit derived from the compound according to claim 1.

19. The resin according to claim 18, having a structure represented by the following formula (2): 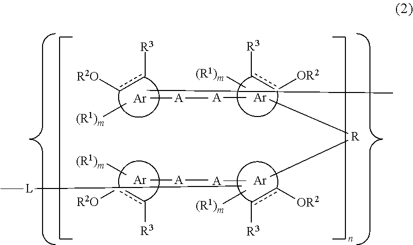
wherein A, R, R¹ to R³, m, n, and p are each as defined in the formula (1); and
L is a single bond or a linking group.

20. (canceled)

21. A composition comprising the compound according to claim 1.

22. (canceled)

23. (canceled)

24. (canceled)

25. (canceled)

26. The composition according to claim 21, which is used in film formation for lithography.

27. The composition according to claim 26, which is used in underlayer film formation for lithography.

28. The composition according to claim 26, which is used in resist film formation.

29. (canceled)

30. (canceled)

31. A resist pattern formation method, comprising:
an underlayer film formation step of forming an underlayer film on a substrate using the composition according to claim 21;
a photoresist film formation step of forming at least one photoresist film on the underlayer film formed through the underlayer film formation step; and
a step of irradiating a predetermined region of the photoresist film formed through the photoresist film formation step with radiation for development.

32. (canceled)

33. A circuit pattern formation method, comprising:
an underlayer film formation step of forming an underlayer film on a substrate using the composition according to claim 21;
an intermediate layer film formation step of forming an intermediate layer film on the underlayer film formed through the underlayer film formation step;
a photoresist film formation step of forming at least one photoresist film on the intermediate layer film formed through the intermediate layer film formation step;
a resist pattern formation step of irradiating a predetermined region of the photoresist film formed through the photoresist film formation step with radiation for development to form a resist pattern;
an intermediate layer film pattern formation step of etching the intermediate layer film with the resist pattern formed through the resist pattern formation step as a mask, to form an intermediate layer film pattern;
an underlayer film pattern formation step of etching the underlayer film with the intermediate layer film pattern formed through the intermediate layer film pattern formation step as a mask, to form an underlayer film pattern; and
a substrate pattern formation step of etching the substrate with the underlayer film pattern formed through the underlayer film pattern formation step as a mask, to form a pattern on the substrate.

34. A method for purifying the compound according to claim 1, comprising:
an extraction step of bringing a solution containing the compound or the resin and an organic solvent that does not inadvertently mix with water into contact with an acidic aqueous solution to carry out extraction.
